(19) United States
(12) Patent Application Publication
(10) Pub. No.: US 2015/0352086 A1
(43) Pub. Date: Dec. 10, 2015

(54) HEAT SHOCK PROTEIN (HSP) INHIBITION AND MONITORING EFFECTIVENESS THEREOF

(71) Applicant: H. LEE MOFFITT CANCER CENTER AND RESEARCH INSTITUTE, INC., Tampa, FL (US)

(72) Inventors: John Matthew Koomen, Tampa, FL (US); Keiran S.M. Smalley, Tampa, FL (US); Elizabeth R. Remily-Wood, Lutz, FL (US); Yun Xiang, Tampa, FL (US)

(21) Appl. No.: 14/759,477

(22) PCT Filed: Jan. 7, 2014
(86) PCT No.: PCT/US14/10476

§ 371 Date: Jul. 7, 2015

Related U.S. Application Data
(60) Provisional application No. 61/749,493, filed on Jan. 7, 2013.

(51) Int. Cl. A61K 31/437; G01N 33/574; A61K 31/46

(52) U.S. Cl. CPC A61K 31/437; A61K 31/46; G01N 33/57484; G01N 2333/47; G01N 2800/7028

(57) ABSTRACT

Provided herein are methods of treating a BRAF inhibitor resistant cancer in a subject. Also provided are methods of monitoring or evaluating the treatment of a cancer in a subject. Further provided are methods of diagnosing a cancer or disease associated with antibody production in a subject.

### CROSS-REFERENCE TO RELATED APPLICATIONS

This application claims benefit of U.S. Provisional Application No. 61/749,493, filed Jan. 7, 2013, which is hereby incorporated herein by reference in its entirety.

### BACKGROUND

BRAF is a member of the Raf kinase family of growth signal transduction protein kinases that plays a role in regulating the MAPK signaling pathway, which has emerged as a central target for cancer therapy due to its persistent activation in the majority of tumors. Since constitutively active BRAF mutants commonly cause cancer by excessively signaling cells to grow, inhibitors of BRAF have been developed for both the inactive and active conformations of the kinase domain as cancer therapeutic candidates. Two of these inhibitors, vemurafenib and dabrafenib are approved by FDA for treatment of late-stage melanoma. BAY43-9006 (Sorafenib, Nexavar) is a V600E mutant BRAF inhibitor approved by the FDA for the treatment of primary liver and kidney cancer. However, their therapeutic success is limited by the emergence of drug resistance, as responses are transient and tumors eventually recur. For example, BRAF mutations are identified in 40-50% of patients with melanoma.

### SUMMARY

Provided are methods of treating a BRAF inhibitor resistant or BRAF mutant cancer in a subject. Intracellular heat shock proteins are highly expressed in cancerous cells and are essential to the survival of these cell types; therefore small molecule inhibitors of HSPs and HSP co-chaperones are effective anticancer agents. The disclosed methods can, for example, comprise administering a pharmaceutically effective amount of a BRAF inhibitor and a HSP inhibitor, HSP co-chaperone inhibitor, or a combination thereof, to the subject. Administration of the BRAF inhibitor, HSP inhibitor, and/or HSP co-chaperone inhibitor to the subject treats the BRAF inhibitor resistant or BRAF mutant cancer in the subject.

There are several HSP inhibitors currently in Phase 1, 2 and 3 clinic trials alone and in combination for cancer indications including IPI-504 (retaspimycin, Infinity), BIIB021 and BIIB028 (Biogen Idec), SNX-5422 (Serenex/Pfizer), AV-142 (Aveo), MPC-3100 (Myriad Genetics), AUY-922 (Vernalis, Novartis), STA-9090 (Synta), KW-2478 (Kyowa Hakko), AT-13387 (Astex) and XL-888 (Exelixis).

In some embodiments, the HSP inhibitor is selected from the group consisting of a HSP90 inhibitor, a HSP27 inhibitor, a HSP70 inhibitor, a HSP71 inhibitor, a HSP72 inhibitor, a HSP74 inhibitor, a HSP7C inhibitor, a HSP7E inhibitor, a HSPA5 inhibitor, a CDC37 inhibitor, and a HSPB3 inhibitor. For example, the HSP90 inhibitor can be selected from the group consisting of a HSP90α inhibitor, a HSP90β inhibitor, and a HSP90β2 inhibitor. Non-limiting examples of HSP90 inhibitors include 5-((R)-sec-butylamino)-N1-((1R,3s,5S)-8-(5-(cyclopropanecarbonyl)pyridin-2-yl)-8-azabicyclo[3.2.1]octan-3-yl)-2-methylterephthalamide (XL888), 17-(Allylamino)-17-demethoxygeldanamycin (17-AAG), 17-Dimethylaminoethylamino-17-demethoxygeldanamycin (17-DMAG), and Ganetespib (STA-9090).

Also provided are methods of monitoring HSP or HSP co-chaperone inhibition in a subject. These methods can involve the use of HSP or HSP co-chaperon inhibition alone, or in combination with a BRAF inhibitor. The methods can comprise administering to the subject a HSP or HSP co-chaperone inhibitor, determining the level of a HSP or HSP co-chaperone in a sample from the subject, and comparing the level of the HSP or HSP co-chaperone to a control. For example, the level of HSP or HSP co-chaperone can be determined using liquid chromatography-multiple reaction monitoring (LC-MRM) or direct infusion-multiple reaction monitoring (DI-MRM) mass spectrometry. In some embodiments, an increase or decrease of the HSP or HSP co-chaperone as compared to a control indicates the presence of HSP or HSP co-chaperone inhibition.

Also provided are methods of predicting responsiveness of a subject with cancer to a heat shock protein (HSP) or HSP co-chaperone inhibitor. These methods can involve determining the level of the HSP or the HSP co-chaperone in a tumor sample from the subject using liquid chromatography-multiple reaction monitoring (LC-MRM) or direct infusion-multiple reaction monitoring (DI-MRM) mass spectrometry. The methods can further involve administering to the subject the HSP or HSP co-chaperone inhibitor if the HSP or HSP co-chaperone is overexpressed in the tumor sample.

Further provided are methods of diagnosing a cancer or disease associated with antibody production in a subject. The methods can comprise obtaining a sample from the subject, determining a level of an antibody in the sample, and comparing the level of the antibody to a control. The level of the antibody can, for example, be determined using DI-MRM. In some embodiments, a comparable level of the antibody as compared to the control indicates the subject has cancer or a disease associated with antibody production.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a diagram of the workflow for LC-MRM assay development. Endogenous proteins of interest were selected; information was gathered to inform assay development, which included the protein sequence, post-translational modification sites, isoforms, and prior Western blots to inform gel excision. Tandem mass spectra were optional, typically due to a lack of availability. Transition lists were created for LC-MRM screening. Lysates were prepared from appropriate cell lines, which were known to contain the protein. The coupling of SDS-PAGE with LC-MRM screening can be used to identify peptides for quantitative assay development. Gel regions were determined by comparison to Western blots and reproducibly cut using the molecular weight markers. When more than 3 peptides were detected in the LC-MRM screens, subsequent assay development was expected to be successful. If 3 peptides were not detected, additional protein may be loaded or immunoprecipitation may be necessary to enrich the protein. After peptide synthesis, the coelution of the endogenous and synthetic peptide and the similarity of the transition ratios were used for verification as a checkpoint to further assay development. LC-MRM screens were completed for 218 proteins that have been selected because of their roles in particular pathways and biological processes listed in Table 1. Synthetic stable-isotope labeled peptide standards have been created for 95 assays to enable relative and absolute quantification.

FIG. 2 shows LC-MRM screens for heat shock proteins. A protein interaction map created using Metacore GeneGO MapEditor was used to show the biological relationships between the different components. Each arrow describes the nature of the interaction using the following codes: green for activation, red for inactivation, gray for other known associations, B for binding, P for phosphorylation, and Cn for competition. For clarity, only selected interactions are shown. FIGS. 2B-2K show LC-MRM screening data, which plot the sum of the ion signal for all transitions in a peptide against time, displayed for each protein in this signaling network. The Roman numeral codes linking the corresponding peptide to each peak are identified in Table 2. These peptide sequences are candidates for quantitative assay development.

FIG. 3 shows data for heat shock protein 90α (HS90A_HUMAN). FIG. 3A shows the sequence (SEQ ID NO:83) of the protein. The sequence of each peptide that passed the initial LC-MRM screen is linked to the corresponding peptide page (as shown here in underlined text). FIG. 3B shows an image of a gel of the protein separation shown with a box indicating the region that was excised for LC-MRM analysis. FIG. 3C shows an example of LC-MS/MS data displayed for each peptide; the fragment ion spectrum obtained on a Q-TOF instrument is shown for peptide, DQVANSAFVER (SEQ ID NO:15). FIG. 3D shows a plot of the LC-MRM screen included to enable ranking of target peptides by ion signal intensity and the selectivity of their transitions. Four peptides were detected: DQVANSAFVER (I) (SEQ ID NO:15), FYEQFSK (II) (SEQ ID NO:14), NPDDITNEEYGEFYK (III) (SEQ ID NO:13), and ALLFVPR (IV) (SEQ ID NO:3). Peptides I and IV were selected for assay development.

FIG. 4 shows an example of standard peptide verification for quantification of HSP90α in RPMI-8226 Myeloma Cells. The coelution of the biological peptide (FIG. 4A) and stable-isotope labeled standard (SIS) (FIG. 4B) was detected at 12.16 minutes for the peptide, ALLFVPR (SEQ ID NO:3). Overlay plots of the ion signals for each transition illustrate similar fragmentation of the endogenous and stable-isotope labeled peptides; the transition ratios match within 2.5%. These data are presented, along with tables of peptide and fragment m/z values, breakdown curves, calibration curve, and a link to the peptide synthesis report, which includes preparative HPLC traces as well as MALDI MS and MS/MS verification of the peptide purity and sequence.

FIG. 5 shows a comparison of ELISA and LC-MRM quantification of HSP90α in RPMI-8226 multiple myeloma cells. Serial dilution curves were created using ELISA (FIG. 5A) and LC-MRM (FIG. 5B) where either absorbance at 450 nm or total peak area of all monitored transitions were plotted against the amount of protein standard analyzed. The amounts of HSP90α protein expressed in RPMI-8226 cells were measured for a range of inputs between 500 and 50,000 cells using both ELISA and LC-MRM (FIG. 4C); the total amount of protein in femtomoles in the sample is plotted against the number of myeloma cells. FIG. 5D shows a correlation of the ELISA and LC-MRM data with equations and r² values for linear fits of the data in each plot.

FIG. 6 shows LC-MRM quantification of HSP Expression in RPMI-8226 multiple myeloma cells before and after treatment with 17-DMAG. Protein expression was measured prior to treatment (0 hours) and 24 hours after administration of 17-DMAG at its IC₅₀. FIG. 6A shows bubble plots of protein expression that enable rapid visual interpretation, similar to “dot” immunoblots or reversed-phase protein array data. FIG. 6B shows a bar graph to illustrate the protein modulation in response to drug and to display the variability in the measurements.

FIG. 7 shows transmission effects at different quadrupole settings. Total fragment ion signal and transition ratios were quantified for the SIS peptide, ALLFVPR (SEQ ID NO:3), after setting different quadrupole resolution values. Modulation of Q1 settings (white bars) was analyzed with Q3 resolution set to 0.7; Q3 resolution values were changed (black bars), while Q1 resolution was set to 0.7. Total fragment ion signal decreased when Q1 resolution was set to lower values (FIG. 7A) and when Q3 resolution is set to lower values (FIG. 7B). Modulation of Q1 did not result in any changes in the ratios between transitions, as illustrated by the comparison of the y₅ (m/z 637) to b₂ (m/z 185) ratios (FIG. 7C). Comparison of the transition ratios between y₅ to y₄ (m/z 524) observed under different Q3 settings indicated no significant differences for fragment ions with similar m/z values (FIG. 7D). However, the ratio of y₅ to b₂ did change when different Q3 resolution values were selected (FIG. 7E), indicating that transition ratios must be measured for each Q3 setting.

FIG. 8 shows protein quantification by Direct Infusion-MRM (DI-MRM). The sensitivity of the assay was determined by examining the transition ratios across a dilution series and comparing with LC-MRM data (FIG. 8A); transition ratios for the AEFVEVTK (SEQ ID NO:24) peptide from BSA were consistent for DI-MRM of concentrations between 0.8 and 80 nM. Peak areas of the endogenous peptide (open circles) and the SIS peptide (closed boxes) indicated competition effects at higher concentrations (FIG. 8B) that illustrate the need for internal standards. Relative quantification across this range showed a strong linear relationship (FIG. 8C).

FIG. 9 shows the DI-MRM transition selection for the HSP90α Peptide, ALLFVPR (SEQ ID NO:3), using LC-MRM Data. Total ion chromatograms (FIG. 9A) for the endogenous (top) and SIS (bottom) peptides indicated that both sets of transitions have interference. The individual transitions are shown for the endogenous peptide (FIG. 9B) and the SIS peptide (FIG. 9C). In both cases, the ion signal for the y₆ fragment ion has the most interference, but the other transitions, y₃, y₄ and y₅ have interference peaks that present between 0 and 38.6% of the total signal. With proper selection and correction factors, these transitions can be used for DI-MRM quantification.

FIG. 10 shows the error in relative and absolute quantification using DI-MRM because of the contribution of interference in transitions for the endogenous and standard peptides. A heat map has been generated to plot the ratio error (%) against the contribution of interference (also in %) in the transitions for the endogenous and standard peptide. If the percentages of interference were equal, the ratio will be unchanged. Calculations for interference levels from LC-MRM data can be mapped on this graph to determine the utility of individual transitions as well as their sums.

FIG. 11 shows the correlation between DI-MRM and LC-MRM quantification of HSPs in digests of whole cell lysate. Correlation between signal intensity in DI-MRM and LC-MRM peak area for all transitions used to monitor HSPs in RPMI-8226 lysates (FIG. 11A). Lower intensity signals have higher DI-MRM intensity values due to noise-broadening. The dashed line indicated the maximum level of LC-MRM noise observed in a solvent blank; however, each transition has a different level of noise (the range is indicated with the arrow). In most cases, the intensity of the noise is ˜1 on this QqQ mass spectrometer. Heat maps of protein expression indicate changes in relative expression of HSPs after treatment with 17-DMAG (FIG. 11B). Expression ratios determined by LC-MRM and DI-MRM were highly correlated, as shown for protein expression in RPMI-8226 cells (FIG. 11C). CV values for DI-MRM measurements for protein expression in RPMI-8226 cells were typically higher than LC-MRM, but the majority (33/40) were still below 20% (FIG. 11D).

FIG. 12 shows the contribution of baseline noise in DI-MRM. Baseline noise was considered in evaluating the ion signals from DI-MRM. Measurements of ion signal intensity for the endogenous transitions for these peptides were used to determine that the noise level on this instrument is typically ≦3 a.u; these values were low most likely due to the ion optics (specifically the S-Lens and the curvature of the second quadrupole). Therefore, the errors induced in relative and absolute quantification based on the contribution of baseline noise have been calculated using three integer values (1, 2, and 3 a.u.) at different levels of endogenous and standard signal. Error was shown for peak intensity values from 5 to 50 a.u.; when the endogenous and standard peptide signal intensity values were both above 100 a.u., error was ≦3%. Given that the endogenous and the standard peptides have similar signals, data for ratio errors were shown for higher noise in endogenous as compared with the standard (FIG. 12A) and vice versa (FIG. 12B). The values in the key were the baseline noise levels of peak intensity as measured in the background (solvent blank) for the transitions of the endogenous and standard peptides, respectively. Additional data were plotted for different levels of endogenous signal, when the standard signal was 100 a.u. As in the first two panels, calculations have been made for higher noise in the endogenous (FIG. 12C) as well as in the standard peptide transition (FIG. 12D). In these data, endogenous signals of 100 a.u. (matching the standard intensity) have errors less than 2% due to the expected levels of noise.

FIG. 13 shows the determination of the optimum acquisition time for analysis of HSP Expression. Transitions (n=184) were monitored in digests of whole cell lysate for 10 minutes. The CV (%) was calculated for each acquisition time using three separate DI-MRM experiments (FIG. 13A). Data for three peptides HSQFLGYPITLYLEK (SEQ ID NO:22) with intensity 22.1 (squares), DQVANSAFVER (SEQ ID NO:15) with intensity 286.3 (circles), and ALLFIPR (SEQ ID NO:4) with intensity 685.0 (triangles) were shown. Using data from two minute acquisitions, all HSP assays had less than 20%. Peak area ratios (endogenous/SIS peptide) from multiple two minute acquisitions from within the same DI-MRM experiment were plotted to evaluate the reproducibility of the quantification (FIG. 13B); assays listed on the x-axis in order of increasing signal. FIG. 13C shows the CV values for two minute acquisitions of each DI-MRM assay plotted against peak intensity to enable determination of the signal required for optimal performance. Data from the peptides used in (FIG. 13A) were indicated by the square, circle and triangle, respectively. Assay CV values were below 10% for all peptides with signal intensities greater than 100.

FIG. 14A to 14H are graphs showing cell survival (%) in the naïve (FIGS. 14A, 14C, 14E, 14G) and resistant (FIG. 14B, 14D, F, 14H) M229 (FIGS. 14A, 14B), WM164 (FIG. 14C, 14D), M249 (FIGS. 14E, 14F), and 1205Lu (FIGS. 14G, 14H) cell lines as a function of dose (log₁₀ scale). FIG. 14I is a graph showing cell survival (%) in naïve (solid line) and resistant (dashed line) cell lines as a function of dose (log₁₀ scale).

FIG. 15 shows the HSP90 inhibitor XL888 blocks the growth and survival of melanoma cell lines with diverse mechanisms of vemurafenib resistance. FIGS. 15A and 15B are graphs showing growth (%) of matched pairs of vemurafenib naïve and resistant melanoma cell lines and melanoma cell lines with intrinsic resistance. In FIG. 15A, cells were treated with increasing concentrations of vemurafenib (1 nM-10 μM: 72 hrs) before being subject to the MTT assay. In FIG. 15B, a cell growth assay shows the response of the cell line panel from FIG. 15A to the HSP90 inhibitor (1 nM-10 μM: 72 hours). FIGS. 15C to 15E are bar graphs showing the cell cycle effects of XL888 (300 nM: 24 hours) upon vemurafenib sensitive and naïve cell lines. Cells were fixed, stained with propidium iodide and distributions analyzed by flow cytometry. FIG. 15F-G are bar graphs showing that XL888 induced apoptosis in every model of acquired vemurafenib resistance tested. Cells were treated for either 72 or 144 hours with XL888 (300 nM) followed by Annexin-V. Apoptosis was measured by flow cytometry.

FIGS. 16A to 16C show that XL888 degraded proteins involved in BRAF inhibitor resistance leading to apoptosis and tumor regression in vivo. FIG. 16A, upper panel shows a colony formation assay demonstrating the long-term effectiveness of XL888. Cell lines were treated with 300 nM XL888 for 4 weeks before being fixed and stained with crystal violet. FIG. 16A, lower panel, shows the quantification of absorbance after 4 weeks of drug treatment. FIG. 16B shows that XL888 degraded IGF1R, PDGFRβ, ARAF, CRAF and cyclin D1 and inhibited pAKT, pERK and pS6 signaling in 4 melanoma cell lines with acquired BRAF inhibitor resistance. XL888 degrades the expression of COT and cyclin D1 in melanoma cell lines with intrinsic resistance to vemurafenib. FIG. 16C is a bar graph showing that combining XL888 with vemurafenib leads to enhanced levels of apoptosis in melanoma cell lines with COT overexpression (RPMI7951). Cells were treated with vemurafenib (3 μM), XL888 (300 nM), or the two in combination for 24-72 hours. Apoptosis was measured by Annexin-V staining and flow cytometry.

FIG. 17 depicts HSP90 client proteins that are important in melanoma.

FIG. 18 shows XL888 was effective at blocking the growth and survival of vemurafenib resistant melanoma cell lines grown as 3D collagen implanted spheroids. Spheroids were grown on top of agar before being implanted into collagen and treated with either vehicle (CT) or XL888 (1 μM) for 144 hours. Magnification ×4.

FIGS. 19A to 19D show the development of a quantitative pharmacodynamic assay for HSP90 inhibition. FIG. 19A shows a work flow diagram of the LC-MRM experiment to measure HSP chaperone levels. After reversed-phase HPLC separation, peptides were selected by their mass-to-charge ratio and dissociated by collisions with background gas before the fragment ions were mass selected to enable specific detection and quantification of individual peptides in complex mixtures. FIG. 19B shows a heatmap demonstrating XL888-induced (0-48 hours, 300 nM) HSP70 expression in all of the melanoma cell lines irrespective of vemurafenib resistance mechanism. FIG. 19C shows an image of a Western blot confirming HSP70 upregulation following XL888 treatment (300 nM, 48 hours). FIG. 19D shows the quantification of absolute (fmol/μg) expression of the HSP chaperone protein expression in fine needle aspirates from two melanoma specimens.

FIGS. 20A to 20C show that XL888 induced the regression of established M229R xenografts and was associated with increased intratumoral HSP70 expression. FIG. 20A shows XL888 led to regression of M229R melanoma xenografts. M229R cells were grown until a palpable tumor had formed before being treated with XL888 thrice per week (100 mg/kg) by oral gavage. The growth curves were normalized to starting volumes. FIG. 20B shows photographs of 3 representative tumors from the vehicle (CT) and XL888 treated groups (XL) at 15 days. XL888 treatment led to significant levels of tumor regression (P=0.003). FIG. 20C shows a heatmap demonstrating the increase in HSP70 isoform 1 (HSP71) expression in XL888 treated (15 days, 100 mg/kg) xenograft samples compared to vehicle controls.

FIGS. 21A to 21D show HSP90 inhibition increased BIM, decreased Mcl-1, and restored apoptosis in vemurafenib-resistant melanoma cell lines. FIG. 21A shows an image of a Western blot demonstrating that XL888 (48 hours, 300 nM) decreased BIM phosphorylation (Ser69) and increased BIM expression. FIG. 21B shows a graph of a quantitative RT-PCR experiment showing that treatment with XL888 (300 nM, 48 hours) increased the expression of BIM at the mRNA level. FIGS. 21C and 21D show siRNA knockdown of BIM significantly decreased XL888 (300 nM, 48 hours) mediated apoptosis in two vemurafenib resistant melanoma cell lines (M229R and 1205LuR). FIG. 21E shows an image of a Western blot of Mcl-1 expression in vemurafenib resistant melanoma cell lines treated with XL888 (300 nM) for 48 hours. FIG. 21F shows a bar graph of a quantitative RT-PCR showing that XL888 (300 nM, 48 hours) treatment downregulated Mcl-1 expression at the mRNA level. FIGS. 21G and 21H show that induction of Mcl-1 reduced the magnitude of XL888 induced apoptosis. FIG. 21G shows an image of a Western blot demonstrating the induction of Mcl-1 following doxycycline treatment. Induction of Mcl-1 (DOX+XL) significantly reduced the level of XL888-induced apoptosis compared to XL888 (XL: 300 nM, 72 hours) alone. *P<0.05

FIG. 22 shows representative images of immunofluorescence staining demonstrating that XL888 (300 nM) enhanced the nuclear accumulation of FOXO3a and increased BIM expression in M229R and 1205LuR cell lines.

FIGS. 23A and 23B show a siRNA knockdown of Mcl-1 induced apoptosis in vemurafenib resistant melanoma cell lines. 1205LuR and M229R cells were treated with either non-targeting (NT) or Mcl-1 siRNA (Mcl-1 si) for 96 hours. Protein was resolved and probed for Mcl-1 and GAPDH expression by Western blot (FIG. 23A). Levels of apoptosis were quantified by Annexin-V staining and flow cytometry (FIG. 23B).

FIGS. 24A to 24D shows that HSP90 inhibition was more effective at restoring the apoptotic response than combined MEK+PI3K inhibition. FIG. 24A shows representative images of immunofluorescence staining of 1205LuR and M229R cells for BIM and FOXO3a following treatment with either vehicle, XL888 (300 nM), AZD6244 (3 μM), GDC-0941 (3 μM) and the combination of AZD6244+GDC-0941 (each at 3 μM). FIG. 24B are bar graphs showing XL888 was more effective than MEK+PI3K inhibitors at increasing BIM and decreasing Mcl-1 mRNA expression in 1205LuR and M229R cell lines. Cells were treated with vehicle, XL888, AZD6244, GDC-0941 and the combination of AZD6244+GDC-0941 (as above) and quantitative RT-PCR was performed on BIM and Mcl-1. FIG. 24C are Western blots showing XL888 was more effective than MEK+PI3K inhibitors at increasing BIM and decreasing Mcl-1 protein expression in 1205LuR and M229R cell lines. Cells were treated with vehicle, XL888, AZD6244, GDC-0941 and the combination of AZD6244+GDC-0941 (as above) and Western blots were performed for BIM and Mcl-1. FIGS. 24D to 24I are bar graphs showing XL888 was more effective at inducing apoptosis of melanoma cell lines where resistance was mediated through COT and PDGFRβ expression and in 2 models where the resistance mechanism was unknown. Cells were treated with XL888, AZD6244, GDC-0941 and AZD6244+GDC-0941 as described above for 72 or 144 hours. Apoptosis was measured by Annexin-V staining and flow cytometry.

FIGS. 25A and 25B are bar graphs showing XL888 (HSPi) was less effective than either an inhibitor of MEK (M229R) or the MEK+PI3K inhibitor (M+P) at inducing BMF mRNA in vemurafenib resistant melanoma cell lines M229R (FIG. 25A) or 1205LuR (FIG. 25B). Cell cultures were treated with either vehicle (CT), HPSi (XL888, 300 nM), MEKi (AZD6244, 3 μM), PI3Ki (GDC-0941, 3 μM) or the MEK+PI3K inhibitor combination for 48 hours, before being analyzed by quantitative RT-PCR.

FIGS. 26A to 26C show that although XL888 degraded the 26S proteasome, it was less effective than the MEK+PI3K inhibitor combination at inhibiting chymotrypsin like proteasome activity. FIG. 26A shows an image of a Western blot demonstrating the degradation of 26S proteasome following XL888 treatment (300 nM, 48 hours). Numbers indicate fold decrease in proteasome activity. FIGS. 26B and 26C are bar graphs demonstrating the quantification of cellular chymotrypsin-like proteasome activity following treatment with either vehicle (CT), XL888 (XL, 300 nM), AZD6244 (AZD, 3 μM), GDC-0941 (GDC, 3 μM), the MEK+PI3K inhibitor combination (A+G) or the proteasome inhibitor MG-132 (1 and 3 μM) in cell lines M229R (FIG. 26V) or 1205LuR (FIG. 26C).

### DETAILED DESCRIPTION

The impressive clinical response of melanoma patients to the BRAF inhibitor vemurafenib is limited by the onset of resistance. Resistance can be intrinsic or acquired; it is mediated through an array of mechanisms including acquired mutations in NRAS and MEK1, overexpression of COT, CRAF, PDGFR-β, cyclin D1 and IGFR1. This apparent diversity of resistance mechanisms, coupled with the phenotypic and cell signaling plasticity of melanoma cells, represents a considerable clinical challenge for which no management strategies currently exist. Here it is demonstrated that all of the signaling proteins implicated thus far in the escape from vemurafenib therapy are clients of heat shock protein (HSP)-90. Inhibition of HSP90 using XL888 overcomes both acquired and intrinsic vemurafenib resistance by restoring the apoptotic response. Therefore, the combination of vemurafenib and an HSP90 inhibitor are useful to delay and/or overcome BRAF inhibitor resistance.

Despite the recent clinical success of BRAF inhibitors like vemurafenib (N-(3-{[5-(4-chlorophenyl)-1H-pyrrolo[2,3-b]pyridin-3-yl]carbonyl}-2,4-difluorophenyl)propane-1-sulfonamide) and dabrafenib (N-{3-[5-(2-aminopyrimidin-4-yl)-2-tert-butyl-1,3-thiazol-4-yl]-2-fluorophenyl}-2,6-difluorobenzenesulfonamide) in BRAF mutant melanoma, most of the responses observed are transient, with relapse and resistance occurring in most cases. The emerging data suggests that BRAF inhibitor resistance is complex, multi-factorial and results from intrinsic and acquired mechanisms. To date, the loss/inactivation of PTEN function, deletion of the retinoblastoma protein (RB), expression of the MAP kinase family member COT and amplification of cyclin D1 have each been shown to mediate intrinsic resistance by either diminishing the apoptotic response or allowing for cell cycle entry when oncogenic BRAF is inhibited. Unlike the acquired drug resistance to imatinib seen in chronic myeloid leukemia and to EGFR inhibitors in non-small cell lung cancer, resistance of melanoma cells to BRAF inhibitors does not result from secondary “gate-keeper” mutations in the BRAF kinase. Instead, acquired resistance has been reported to be mediated through constitutive signaling by receptor tyrosine kinases (RTKs) (IGF1R and PDGFR-β), mutations in NRAS or MEK1, or by the increased expression of COT. The apparent diversity of resistance mechanisms, and the likelihood that others exist is expected to complicate the design of future clinical trials to prevent or treat resistance to BRAF inhibitors. The heat shock protein (HSP)-90 family of chaperones maintains the malignant potential of cancer cells by regulating the conformation, stability and function of many RTKs and kinases required for oncogenic transformation. Many proteins required for melanoma initiation and progression, including mutated BRAF, CRAF, IGF1R, cyclin D1, CDK4 and AKT are known to be clients of HSP90.

Provided are methods of treating a BRAF inhibitor resistant or BRAF mutant cancer in a subject. The methods can, for example, comprise administering a pharmaceutically effective amount of a BRAF inhibitor and a heat shock protein (HSP) inhibitor to the subject. The methods can, for example, comprise administering a pharmaceutically effective amount of a BRAF inhibitor and a heat shock protein (HSP) co-chaperone inhibitor to the subject. Administration of the BRAF inhibitor, HSP inhibitor, and/or HSP co-chaperone inhibitor to the subject treats the BRAF inhibitor resistant or BRAF mutant cancer in the subject.

A BRAF inhibitor resistant cancer can, for example, be a cancer that has developed a resistance to treatment with a BRAF inhibitor. Resistance can be intrinsic or acquired and can be mediated through an array of mechanisms, e.g., mutations in NRAS, MEK1, overexpression of COT, CRAF, PDGFR-β, cyclin D1, and/or IGFR1. A BRAF mutant cancer is a cancer caused by a mutation in BRAF. BRAF mutant cancers are known in the art, see, e.g., Dienstmann and Tabernero, Anticancer Agents Med. Chem. 11(3):285-95 (2011).

The HSP inhibitor can, for example, be selected from the group consisting of a HSP90 inhibitor, a HSP27 inhibitor, a HSP70 inhibitor, a HSP71 inhibitor, a HSP72 inhibitor, a HSP74 inhibitor, a HSP7C inhibitor, a HSP7E inhibitor, a HSPA5 inhibitor, and a HSPB3 inhibitor. Optionally, the HSP inhibitor is a HSP90 inhibitor. A HSP90 inhibitor can, for example, be selected from the group consisting of a HSP90α inhibitor, a HSP90β inhibitor, and a HSP90β2 inhibitor. A HSP90 inhibitor can, for example, be selected from the group consisting of XL888, 17-AAG, 17-DMAG, and STA-9090. Optionally, the HSP90 inhibitor is XL888. HSP90 inhibitors are known in the art, see, e.g., Petrikaite and Matulis, Medicina: 47(8):413-20 (2011); Lu et al., Biochem. Pharmacol. (2011); Jhaveri et al., Biochim. Biophys. Acta (2011); Richardson et al., Br. J. Haematol. 152(4):367-79 (2011); Georgakis and Younes, Future Oncol. 1(2):273-81 (2005); and Wang et al., Curr. Opin. Investig. Drugs 11(12):1466-76 (2010).

A HSP co-chaperone inhibitor can, for example, be selected from the group consisting of a HSP90 co-chaperone inhibitor, a HSP27 co-chaperone inhibitor, a HSP70 co-chaperone inhibitor, a HSP71 co-chaperone inhibitor, a HSP72 co-chaperone inhibitor, a HSP74 co-chaperone inhibitor, a HSP7C co-chaperone inhibitor, a HSP7E co-chaperone inhibitor, a HSPA5 co-chaperone inhibitor, a CDC37 co-chaperone inhibitor, and a HSPB3 co-chaperone inhibitor. Optionally, the HSP-co-chaperone inhibitor is a HSP90 co-chaperone inhibitor. The HSP90 co-chaperone inhibitor is selected from the group consisting of a HSP90α co-chaperone inhibitor, a HSP90β co-chaperone inhibitor, and a HSP90β2 co-chaperone inhibitor.

The BRAF inhibitor can, for example, be selected from the group consisting of vemurafenib and dabrafenib. Optionally, the BRAF inhibitor is vemurafenib. BRAF inhibitors are known in the art, see, e.g., Fedorenko et al., Biochem. Pharmacol. 82(3):201-9 (2011) and Puzanov et al., Mol. Oncol. 5(2):116-23 (2011).

A BRAF inhibitor resistant cancer as defined herein, refers to a cancer that is due to a mutation in BRAF (or other intrinsic or acquired mechanisms) that has become resistant to a BRAF inhibitor. A BRAF inhibitor resistant cancer can, for example, be selected from the group consisting of a multiple myeloma, lung cancer, colorectal cancer, thyroid carcinoma, blood cancer, leukemia, and lymphoma. Optionally, the BRAF inhibitor resistant cancer is a melanoma.

Quantitative proteomics is coming of age with rapid advances in liquid chromatography (LC) coupled to selected reaction monitoring mass spectrometry; the assessment of panels of analytes has been termed multiple reaction monitoring (LC-MRM). Prior literature has focused on panels of specific biomarkers, typically for detection in plasma. Strategies have also been developed that the use shotgun sequencing for protein discovery prior to the development of targeted quantitative assays for peptides that are differentially detected. Proteome-wide initiatives are currently underway, including translation of shotgun sequencing data into quantitative assays, as well as broad-scale assessment of MRM transitions using triggered tandem mass spectra to develop peptide targets for the quantification of a significant portion of the yeast proteome. This technology holds great promise for elucidation of cancer biology and patient assessment; LC-MRM is being adopted into many clinical and translational research programs.

Liquid chromatography-multiple reaction monitoring mass spectrometry (LC-MRM) has emerged as versatile method for quantification of protein biomarkers via tryptic peptide surrogates. LC-MRM has been implemented for measurement of biomarker panels, modified peptides, and detection of mutations. LC-MRM has become the method of choice for evaluation of candidate biomarkers generated by discovery proteomics; in addition, standardization has enabled reproducible data to be generated at multiple sites. However, most LC-MRM experiments have low to medium throughput (10s of samples per day). To develop additional capability for high throughput protein quantification, direct infusion (DI) can be used for sample introduction prior to MRM analysis. This development may augment the current LC-MRM capabilities in biomarker evaluation and biology.

Similar to the development trajectory of LC-MRM, direct infusion mass spectrometry has been well established in quantitative analysis of small molecules. Quantification of drugs using chip-based nanoelectrospray ionization sample introduction has also been evaluated for regulatory compliance, indicating that these methods can be translated to clinical scenarios. In addition, “top-down” proteomics approaches often use direct infusion nanospray ion sources. Applications have also been demonstrated in “bottom-up” or peptide-based proteomics. The use of infusion nanospray will complement matrix assisted laser desorption ionization (MALDI) MRM assay development for quantification of small molecules and proteins.

Also provided are methods of monitoring heat shock protein (HSP) or HSP co-chaperone inhibition in a subject. The methods comprise administering to the subject with cancer a HSP or HSP co-chaperone inhibitor, determining the level of a HSP or HSP co-chaperone in a sample from the subject, and comparing the level of the HSP or HSP co-chaperone to a control. The level of HSP or HSP co-chaperone can be determined using liquid chromatography-multiple reaction monitoring (LC-MRM) or direct infusion-multiple reaction monitoring (DI-MRM) mass spectrometry. An increase or decrease of the HSP or HSP co-chaperone as compared to a control indicates the presence of HSP or HSP co-chaperone inhibition.

Optionally, the subject has a disease or condition associated with altered levels of HSP or HSP co-chaperones. Optionally, the disease or condition is cancer. Monitoring HSP or HSP co-chaperone inhibition to determine the presence of inhibition can, for example, determine whether the cancer is being treated. By way of an example, administration of a HSP90 inhibitor can lead to an increase in the level of HSP70, thus an increased level of HSP70 as compared to a control indicates HSP90 inhibition and can indicate treatment of the cancer. By way of another example, if administration of the HSP90 inhibitor does not result in an increased level of HSP70, then HSP90 inhibition is not occurring and this indicates a need for a change in the cancer treatment regimen (e.g., an increased level of HSP or HSP co-chaperone inhibitor could be administered to the subject, or, alternatively, a different treatment method could be pursued).

As used herein, a control can, for example, be determined from a previous sample of the subject. The level of the HSP or HSP co-chaperone can be determined from a sample that is isolated prior to the administration of the HSP or HSP co-chaperone inhibitor. Optionally, if the subject has cancer, the level of the HSP or HSP co-chaperone can be determined from a sample taken from the subject prior to the diagnosis of the cancer. A control can, for example, also refer to a known standard. A person of skill in the art is capable of determining the proper control to evaluate the level of the HSP or HSP co-chaperone after administration of the HSP or HSP co-chaperone inhibitor. If the levels of the HSP or HSP co-chaperone are not increasing or decreasing as desired, the level of the HSP or HSP co-chaperone inhibitor can be increased or decreased as deemed necessary by a person skilled in the art.

The HSP can, for example, be selected from the group consisting of HSP90, HSP27, HSP70, HSP71, HSP72, HSP74, HSP7C, HSP7E, HSPA5, CDC37, and HSPB3. The HSP90 can, for example, be selected from HSP90α, HSP90β, or HSP90β2. The HSP co-chaperone can, for example, be a co-chaperone of any of the above-mentioned HSPs, e.g., the co-chaperone can be selected from the group consisting of a HSP90 co-chaperone, a HSP27 co-chaperone, a HSP70 co-chaperone, a HSP71 co-chaperone, a HSP72 co-chaperone, a HSP74 co-chaperone, a HSP7C co-chaperone, a HSP7E co-chaperone, a HSPA5 co-chaperone, a CDC37 co-chaperone, and a HSPB3 co-chaperone. The HSP co-chaperone can, for example, be a HSP90 co-chaperone. Optionally, the HSP90 co-chaperone is a HSP90α, HSP90β, or HSP90β2 co-chaperone. Optionally, the HSP client proteins consist of IGFR1, COT, AKT, ARAF, MEF, CRAF, cyclin D1, and PDGFR-β could also be measured.

Optionally, determining a level of the HSP or HSP co-chaperone comprises determining a level of a HSP or HSP co-chaperone polypeptide fragment. Optionally, the HSP or HSP co-chaperone polypeptide fragment is selected from any one of SEQ ID NOs:3-16, SEQ ID NOs:18-20, and SEQ ID NOs:26-69.

The cancer can, for example, comprise any cancer in which the level of a HSP or HSP co-chaperone has been altered as compared to a non-cancerous level of HSP or HSP co-chaperone. Optionally, the cancer is selected from the group consisting of multiple myeloma, melanoma, lung cancer, colorectal cancer, blood cancer, leukemia, and lymphoma.

Further provided are methods of diagnosing a cancer or disease associated with antibody production in a subject. The methods comprise obtaining a sample from the subject, determining a level of an antibody in the sample, and comparing the level of the antibody to a control. The level of the antibody can, for example, be determined using DI-MRM. A comparable level of the antibody as compared to the control indicates the subject has cancer or a disease associated with antibody production.

In current clinical practice, the presence of monoclonal immunoglobulin is detected or quantified by serum protein electrophoresis (SPEP) and the total amount of the immunoglobulin is quantified by nephelometry. In reaction monitoring mass spectrometry, specific structural fragments are isolated from specific peptide precursors and quantified by the integration of their peak area. Each precursor and fragment pair is termed a transition. Several transitions detected at the same time provide confidence in the quantification of the molecule.

A level of antibody can, for example, be determined by comparing to a reference standard of known values. A person of skill in the art is capable of preparing a reference standard of known values. The reference standard can be prepared at the same time, prior to, or after determination of the level of antibody. For MRM mass spectrometry, these standards are typically stable-labeled peptides but peptides that are structurally analogous to the sequence of interest can also be used if stable-labeled peptides are not available. The signals for the endogenous (biological) peptide are compared to the same set of signals from the standard peptide to enable quantification.

As used herein a control can comprise a known value or reference sample. A known value refers to a value from a diseased sample or a group of diseased samples, which can represent, a sample from a subject diagnosed with cancer or a disease associated with antibody production. Optionally, the reference sample is from a diseased subject of similar size, weight, height and gender, as the subject being tested.

Optionally, the antibody comprises a heavy chain selected from the group consisting of IgG, IgA, IgM, IgD, and IgE. Optionally, the antibody comprises a light chain selected from a kappa light chain or a lambda light chain.

The level of the antibody can, for example, be determined by determining a level of an antibody polypeptide fragment. Optionally, the antibody polypeptide fragment is selected from one of SEQ ID NOs:70-81.

The cancer can, for example, comprise any disease in which the level of an antibody has been altered as compared to a non-cancerous level of an antibody. Optionally, the cancer is selected from the group consisting of multiple myeloma, melanoma, lung cancer, colorectal cancer, blood cancer, leukemia, and lymphoma. The disease or condition associated with antibody production can be selected from plasma cell dyscrasias or monoclonal gammopathy of undetermined significance (MGUS). Additionally, these measurements may also be relevant to patients with immunodeficiency, including HIV or AIDS.

Provided herein are methods of treating cancer in a subject. Such methods include administering a pharmaceutically effective amount of a BRAF inhibitor, a HSP inhibitor, and/or a HSP co-chaperone inhibitor. Optionally, the BRAF inhibitors, HSP inhibitors, and/or HSP co-chaperone inhibitors are contained within a pharmaceutical composition.

Provided herein are compositions containing the provided BRAF inhibitors, HSP inhibitors, and/or HSP co-chaperone inhibitors and a pharmaceutically acceptable carrier described herein. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippincott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the BRAF inhibitors, HSP inhibitors, and/or HSP co-chaperone inhibitors, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism, for example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout a biological sample from the subject can include a fluid or tissue composition obtained from the subject. Biological samples can, for example, include, but are not limited to, whole blood, peripheral blood, blood plasma, bone marrow, spleen, serum, urine, tears, saliva, sputum, exhaled breath, nasal secretions, pharyngeal exudates, bronchoalveolar lavage, tracheal aspirations, interstitial fluid, lymph fluid, meningeal fluid, amniotic fluid, glandular fluid, feces, perspiration, mucous, vaginal or urethral secretion, cerebrospinal fluid, and transdermal exudate. A biological sample also includes experimentally separated fractions of all of the preceding solutions or mixtures containing homogenized solid material, such as feces, tissues, and biopsy samples.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., cancer or disease associated with antibody production). The term patient or subject includes human and veterinary subjects.

A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder.

According to the methods taught herein, the subject is administered an effective amount of the agent (e.g., a BRAF inhibitor, a HSP inhibitor, and/or a HSP co-chaperone inhibitor). The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Liquid Chromatography-Multiple Reaction Monitoring (LC-MRM) Mass Spectrometry Assays for Elucidating Therapeutic Response in Cancer

Materials and Methods

Reagents.

Chemicals are purchased from Sigma-Aldrich (St. Louis, Mo.) at their highest available purity unless otherwise noted. HPLC solvents (water and ACN) are supplied by Burdick and Jackson (Honeywell, Muskegon, Mich.). Stable-isotope-labeled FMOC amino acids were purchased from Cambridge Isotope Laboratories (Andover, Mass.).

Cell Culture, Lysis, and Sample Preparation for MS.

Cell lines were obtained from the American Type Culture Collection (ATCC, Manassas, Va.) and cultured as directed. Typical conditions are RPMI 1640 medium supplemented with 10% heat-inactivated FCS, with incubation in a 5% CO₂ atmosphere at 37° C. Cell lines are included in Table 1.

The detection of 100 attomoles of yeast alcohol dehydrogenase I (ADH1_YEAST) against a background of lysate from RPMI-8226 myeloma cells was used to establish the sensitivity as well as the variability of quantification of low amounts of protein from biological samples. Triplicate samples of 500 attomoles of ADH1 were spiked into lysate prepared from 10⁵ cells from the RPMI-8226 multiple myeloma line (˜10 mg of the total protein). This number of cells was selected because it is at least two-fold higher than the experiments usually use. Proteins were fractionated with SDS-PAGE prior to gel excision (based on the migration of higher amounts of the standard protein in adjacent lanes), reduction, alkylation, trypsin digestion, and LC-MRM analysis of 20% of the sample (100 attomoles injected). A control of RPMI-8226 cell lysate without ADH1 was analyzed in parallel to enable detection of interference. Each LC-MRM analysis was repeated three times. Two peptides were used to measure the amount of the protein: ANELLINVK (SEQ ID NO:1) (m/z 507.3), and DIVGAVLK (SEQ ID NO:2) (m/z 407.8). Each peptide was quantified using an internal standard, which contained a stable-isotope label: ANELLINV(¹³C₅¹⁵N₁)K (SEQ ID NO:1) (m/z 510.3) and DIVGAVL(¹³C₆¹⁵N¹)K (SEQ ID NO:2) (m/z 411.3).

For LC-MRM screens, cells were lysed in aqueous 8M urea/100 mM ammonium bicarbonate buffer on ice or aqueous 50 mM Tris-HCl pH 7.4, 0.1% NP-40, 1M NaCl, protease inhibitor cocktail (Roche; Indianapolis, Ind.), 25 mM NaF, 2 mM Na₃VO₄, and 0.1M Na₂HPO₄. After lysis, the cell supernatants were clarified by centrifugation and decanted. The equivalent of 25,000-100,000 cells or 2.5-mg of protein was loaded for SDS-PAGE separations, using 10% or 4-12% Criterion XT Bis-Tris gels, which was then visualized with colloidal Coomassie Brilliant Blue G-250 (Bio-Rad, Hercules, Calif.).

Because the bands corresponding to the endogenous proteins of interest were not specifically visualized (as they would be in immunoprecipitates), gel regions containing proteins of interest were excised using the molecular weight (MW) markers as the first guide and, if necessary, by banding patterns observed for the whole cell lysate. For LC-MRM screens, the MW markers were used exclusively and adjacent gel regions were also screened for the endogenous protein of interest. Proteins were reduced with 2 mM Tris-carboxyethylphosphine (TCEP) and alkylated with 20 mM iodoacetamide (IAA) prior to in-gel digestion with sequencing grade trypsin (Promega, Madison, Wis., USA). Following digestion at 37° C. overnight, samples were concentrated by vacuum centrifugation. The peptides were resuspended in 2% acetonitrile (ACN) with 0.1% formic acid (LC solvent A) for mass analysis.

Peptide and Transition Selection for LC-MRM Screening.

The peptides and transitions were predicted by SRM Builder (Prakash et al., J. Proteome Res. 8:2733-9 (2009)), now called Pinpoint (Thermo, San Jose, Calif.) or Skyline (MacLean et al., Bioinform. 26:966-8 (2010)). Doubly protonated molecules corresponding to all peptides between 7 and 25 amino acids in length were probed; cysteine and methionine containing peptides were excluded unless there were few other choices (n<5). In addition, each peptide was reviewed using the existing literature and online databases to examine whether MS/MS data were available and if the sequence contains sites of mutation or post-translational modification. Protein isoforms were also examined to determine peptides that are common in all sequences and to identify peptides that were unique to each isoform. Then, all y fragment ions for each peptide starting from y₃ or yₓ>peptide m/z and ending with y₍ₙ₋₁₎ were monitored in the initial screens. Methods were developed for Xcalibur 2.0 SR2 and TSQ Quantum 1.4 software versions; no scheduling was used for LC-MRM screening. To date, this strategy has not been limiting. For high-molecular-weight proteins, one experiment was used to monitor the best candidates; for lower molecular weight proteins with fewer peptide candidates, the screening was multiplexed. Therefore, each screen contained less than 140 transitions (conservatively 28 peptides with 5 fragment ions each) at 20 milliseconds sampling and still obtained at least 7 points in each peak. Because the peptides were limited to those expected to be optimal for LC-MRM based on length, amino acid content, and flanking sequences, scheduling or multiple screens for any protein have not been necessary.

LC Coupled to MRM.

A nanoflow liquid chromatograph (U3000, Dionex, Sunnyvale, Calif. or Easy-nLC, Proxeon, Denmark) was coupled with the triple quadrupole mass spectrometer (TSQ Quantum Ultra, Thermo, San Jose, Calif.). Aliquots of each sample were loaded onto a C18 reversed-phase trap column and washed for 20 minutes, prior to switching in line with C18 analytical column (PepMap100, Dionex) with 75 mm inner diameter, 15 cm length, 3 mm particle size, and 100 Å pore size. Peptides were eluted at 300 nL/minute using a 35 minute gradient from 5% B to 50% B. The solvent system was composed of aqueous 2% ACN with 0.1% formic acid (A) and aqueous 90% ACN with 0.1% formic acid (B). LC-MRM was performed with 2500 V nanoelectrospray from 10 mm tips (New Objective, Woburn, Mass.) with 200° C. transfer tube temperature, and 12 V skimmer offset. The Q1 setting is m/z 0.2 or m/z 0.4 in width, and Q3 is set to filter m/z 0.7 in width. Fragmentation was achieved with 1.5 mTorr argon. Each transition was monitored for 20 milliseconds. Consistent coelution of transitions and ratios of signal intensity were used to verify peptide candidates; these data were compared with LC-MS/MS data, when available. Currently, one peptide standard was synthesized for quantification of each protein's expression, but all peptides detected in these screens can be monitored in biological experiments. Additional peptides were being synthesized to improve the quantification of expression and enable the assessment of post-translational modifications.

Peptide Synthesis and Evaluation.

Solid-state peptide synthesis (Symphony, Protein Technologies, Tucson, Ariz.) was used to make standards at the 25 mmole scale using standard FMOC chemistry. Purified peptides were analyzed with MALDI-MS to verify coupling efficiency and stable-isotope incorporation level (as specified by Cambridge Isotope Laboratories) and sequenced with MS/MS (4700, Applied Biosystems, Framingham, Mass.). Peptides in 2% ACN with 0.1% formic acid are mixed 1:1 with CHCA (10 mg/mL) in 50% ACN and deposited in 1 mL aliquots on the MALDI target. Peptide standards were quantified by amino acid analysis using 6N HCl-phenol hydrolysis; amino acids were derivatized by FMOC and OPA (to label both primary and secondary amino acids) and then detected after reversed-phase HPLC separation (Amino-Quant, Hewlett Packard; Palo Alto, Calif.).

Verification of Coelution and Transition Patterns Comparing Endogenous and Standard Peptides.

Biological samples were prepared from cell lysates as described above. Stable-isotope-labeled internal standards were added to the samples after in-gel digestion, but prior to vacuum centrifugation, for triplicate LC-MRM analyses. The coelution of the peptide standard and the endogenous peptide of interest and the similarity of their composite tandem mass spectra were used to verify the identity of the peptide and its utility in the assay.

Comparison of Absolute Quantification by ELISA and LC-MRM.

ELISA assays were performed in 96-well plates for expression of HSP90α according to the manufacturer's instructions (EKS-895, Assay Designs-Stressgen, Ann Arbor, Mich.). This sandwich assay uses horseradish peroxidase modification of the tetramethylbenzidine substrate for colorimetric monitoring at 450 nm using a microplate reader (Versa Max, Molecular Devices, Sunnyvale, Calif.). Serial dilutions of the provided protein standard were analyzed with ELISA to make a standard curve. In addition, aliquots of the standard were denatured and digested in solution prior to LC-MRM quantification to create a calibration curve for the peptide-based assay. Then, the amounts of HSP90α were measured in RPMI-8226 multiple myeloma cells (n=500 to 50,000 or 50 ng to 5 μg of protein) using both methods. For ELISA, cells were lysed in the buffer provided by the kit. For LC-MRM, cells were lysed in 8M urea with 30 mM ammonium bicarbonate. After reduction with TCEP and alkylation with iodoacetamide (IAA), the clarified lysate was diluted tenfold and digested with trypsin. LC-MRM analysis was performed for the peptide, ALLFVPR (SEQ ID NO:3), using Q1 to select peptides with a width of m/z 0.4 and Q3 filtering fragment ions with a width of m/z 0.7. Quantification was performed using the stable isotope-labeled peptide, ALLFVP(¹³C₅¹⁵N₁)R (SEQ ID NO:3), spiked in at a known concentration. Other instrument parameters were identical to those described above.

Monitoring HSPs in Multiple Myeloma Cells.

RPMI-8226 multiple myeloma cells were cultured in suspension as described above with the addition of 100 units/mL penicillin/streptomycin. Stock 17-dimethylaminoethylamino-17-demethoxy-geldanamycin (17-DMAG) was dissolved in DMSO at a concentration of 10 mM. Based on prior IC₅₀ measurements, the RPMI-8226 cells were treated with 106 nM 17-DMAG for 24 hours.

RPMI-8226 cells (n=10⁶) were lysed using 50 mL aqueous 8M urea with 100 mM ammonium bicarbonate. Protein concentration was measured by the Bradford assay and 0.38 μg of protein (equivalent to 3,750 cells) was injected for LC-MRM analysis of control and drug-treated samples. The proteins were reduced with 2 mM TCEP, alkylated with 20 mM IAA, and digested overnight in solution with trypsin at 37° C. Corresponding stable-isotope-labeled internal standard peptides were added to the samples after digestion. Then, peptides were extracted using pipette tips packed with C, 18 reversed-phase resin (Ziptip, Millipore; Billerica, Mass.) and concentrated to dryness by vacuum centrifugation (Speedvac, Thermo), and resuspended in aqueous 2% ACN with 0.1% formic acid prior to MS. LC-MRM was performed as described above. Quantification is achieved by using the sum of the peak areas for all detected transitions using QuanBrowser (Xcalibur, Thermo, San Jose, Calif.) or Skyline. The ratio of peak area of the endogenous peptide over corresponding internal standard was calculated for each peptide in the sample to enable absolute and relative quantification. Relative protein expression after treatment was normalized to the pretreatment control for each cell line and plotted to show the change in expression after drug treatment. Bubble plots were created using the SigmaPlot 10 (SPSS).

Results

Target Protein Selection.

A list of representative signaling pathways and biological processes targeted for assay development were prepared. These clusters of interacting proteins were chosen due to relevance to numerous cancer models (Hanahan and Weinberg, Cell 100:57-70 (2000)); frequently, these individual proteins do not have high-quality antibodies or antibody-based assays available for detection and quantification. Most importantly, these proteins represent drug targets, signaling pathways, and biological processes that are well characterized in cancer biology; quantitative assays could be used to translate basic science hypotheses into clinical research with the ultimate goal of patient assessment (Chen et al., J. Proteome Res. 9:4215-27 (2010); Zhang et al., J. Proteome Res. PMID:21080693 (2010)). LC-MRM peptide-based assays can be used for quantifying panels of proteins, enabling multiplexed analysis of protein expression and modification across entire pathways and processes, rather than just focusing on the hubs of protein interaction and activity. This database can also indicate when proteins are interactive in multiple pathways, e.g. HSP90 proteins in heat shock response and EGFR signaling.

Strategy.

The coupling of SDS-PAGE with LC-MRM was selected for numerous reasons, many of which are based on the experiments combining SDS-PAGE and MS in protein quantification and proteome cataloging. For assay development, lysates were prepared from cancer cell lines that were known to contain the protein of interest from prior Westerns, which can be used to predict the migration of the protein. The coupling of SDS-PAGE with LC-MRM enables cancer biologists to view this technology as a direct complement to or enhancement of Westerns. While the sensitivity of SDS-PAGE coupled to LC-MRM has been demonstrated to be sufficient for attomole level detection of proteins (Gerber et al., PNAS USA 100:6940-5 (2003)), additional data were acquired for replicates of 100 attomoles of alcohol dehydrogenase I from yeast (ADH1_YEAST) spiked into a background of lysate from RPMI-8226 multiple myeloma cells prior to SDS-PAGE. These data also illustrate that SDS-PAGE coupled with LC-MRM was a sensitive technique for protein detection. Furthermore, in-gel digestion and subsequent peptide recovery were consistent, because coefficient of variation (CV) values were 10.7% for measuring ANELLINVK (SEQ ID NO:1) and 7.1% for measuring DIVGAVLK (SEQ ID NO:2). Furthermore, the ease of use, reproducibility, and tolerance for contamination (e.g. buffers, salts, and detergents) make SDS-PAGE a methodology that can be implemented in every lab, enabling each collaborating investigator to prepare samples for LC-MRM. The molecular weight fractionation reduced potential interferences for selected transitions and is also useful to isolate isoforms or active/inactive proteins (e.g. NFκB and Notch).

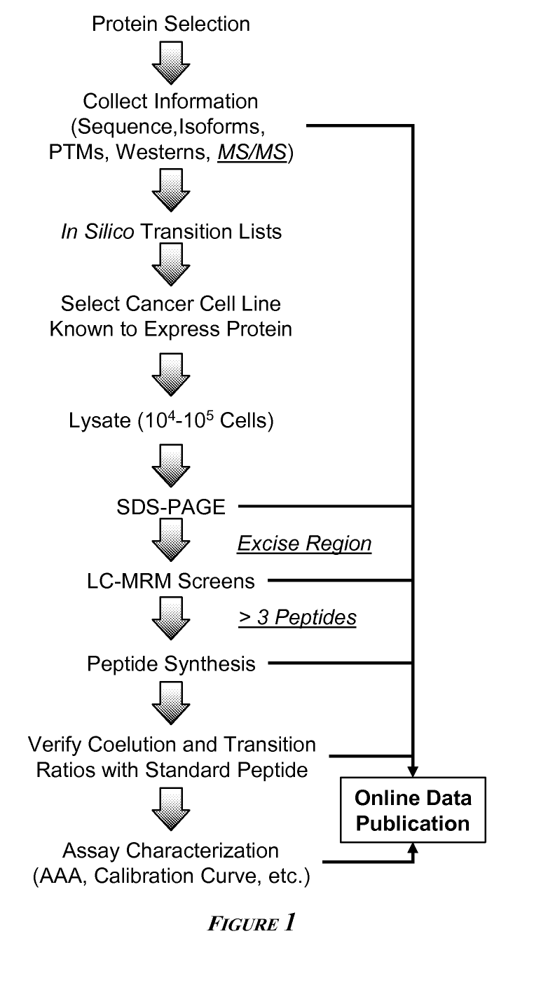

Tryptic digests of gel bands or gel regions delineated by the MW markers were then probed with LC-MRM screens to identify peptides that could be used to quantify the protein of interest, as shown in the workflow (FIG. 1). LC-MRM screening of SDS-PAGE-separated lysates showed few false positives in determining appropriate peptides for monitoring the low-abundance, hypothesis-driven proteins targeted to date. It was rare that the retention time and fragmentation pattern of synthetic stable-isotope-labeled peptides did not match the data for the endogenous peptide observed in the LC-MRM screen.

Pathway Displays.

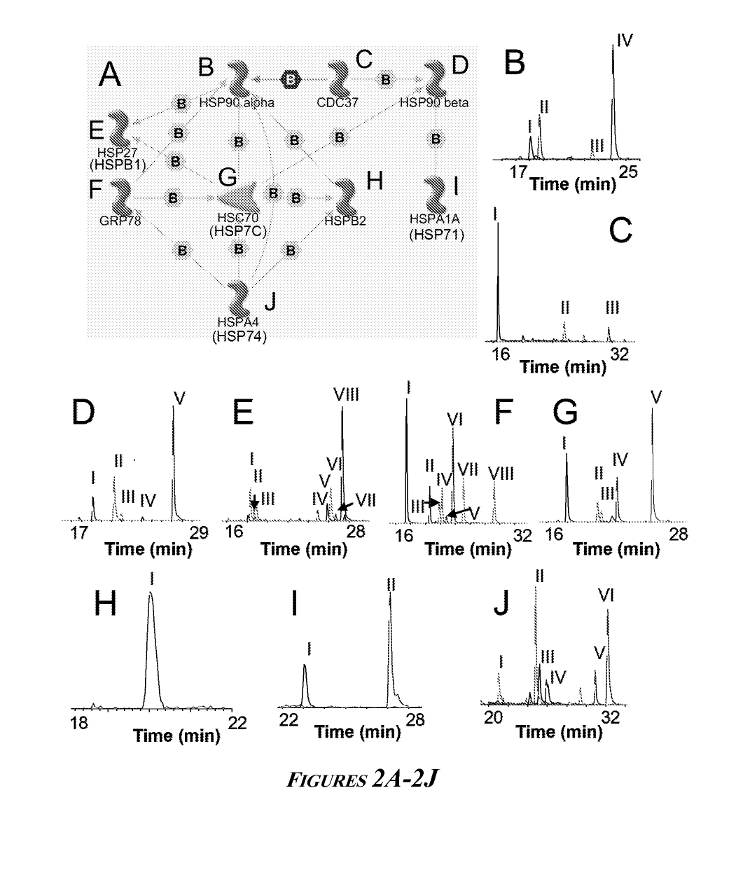

Protein interaction networks were displayed using MapEditor from GeneGO, which demonstrated the relationships between the target proteins. Components of the heat shock response and protein chaperoning process were illustrated as an example of the pathway maps in the QuAD (FIG. 2A). Because of their role in folding and chaperoning proteins (particularly mutant forms), the HSPs have emerged as promising drug targets in diverse cancer types, including multiple myeloma, leukemias, and solid tumors. Phase 1 clinical trials have recently been completed for the HSP90 inhibitor, 17-DMAG. The ability to monitor numerous components of the chaperone network will enable the systematic observation of the response to HSP treatment and detect changes indicative of drug sensitivity or drug resistance.

The main purpose of the pathway maps was to define the clusters of interacting proteins and show how multiplexed assays could be constructed and implemented to address biological hypotheses. In addition to the map, a table listing the protein icons in the pathway map with their corresponding names and UniProt accession numbers is provided at the bottom of the webpage. Each protein icon in the map is linked to the corresponding webpage with data for that protein target. Here, the results of the LC-MRM screens are shown for each protein (FIGS. 2B-2J); peptides selected for assay development are listed in Table 1. For the initial analysis of HSPs and other protein chaperones, the following proteins were selected based on the literature curation: HSP90α (HS90A_HUMAN), HSP90β (HS90B_HUMAN), HSP90β2 (H90B2_HUMAN), the inhibitory co-chaperone CDC-37 (CDC37_HUMAN), HSP27 (HSPB2_HUMAN), HSPβ3 (HSPB3_HUMAN), HSP70 isoform 1 (HSP71_HUMAN), HSP70 isoform 2 (HSP72_HUMAN), HSP70 isoform 4 (HSP74_HUMAN), heat shock cognate 71 (HSP7C_HUMAN), and heat shock 70 kDa protein 5 (GRP78_HUMAN). Potential peptide matches detected by the screens are reported in the online database; peptides chosen for assay development are listed in Table 1.

TABLE 1
Endogenous peptides, corresponding internal standards, and selected
transitions for quantification of heat shock proteins (HSPs) in
RPMI-8226 multiple myeloma cells. Underlined amino acid residues
are stable-isotope labeled with ¹³C and ¹⁵N in the corresponding
synthetic standards. Student's t-tests were used for calculation
of p values. Roman numerals indicate the corresponding ion signals
in FIG. 2, panels B-J.

| | | | Pre- | Post- | | |
|---|---|---|---|---|---|---|
| Protein | | | Treatment | Treatment | | |
| (UniProt | Endogenous | | (amol/ | (amol/ | | p |
| Ident.) | Peptide | Transitions | cell) | cell) | Ratio | value |
| HS90A | ALLFVPR (IV) | y₃-y₆ | 11.2 | 20.9 | 1.87 | 2.9E−5 |
| | (SEQ ID NO: 3) | | | | | |
| HS90B | ALLFIPR (V) | y₃-y₆ | 7.90 | 14.5 | 1.84 | 4.4E−4 |
| | (SEQ ID NO: 4) | | | | | |
| H90B2 | HSQFLGYPITLYLEK (IV) | y₃-y₁₂ | 16.1 | 25.3 | 1.57 | 3.2E−4 |
| | (SEQ ID NO: 5) | | | | | |
| CDC37 | LQAEAQQLR (I) | y₄-y₇ | 1.08 | 0.91 | 1.07 | 0.038 |
| | (SEQ ID NO: 6) | | | | | |
| HSPB3 | ADLINNLR (I) | y₃-y₇ | 0.11 | 0.14 | 1.25 | 0.026 |
| | (SEQ ID NO: 7) | | | | | |
| HSP71 | NQVALNPQNTVFDAK (I) | y₃, y₄, | 0.013 | 0.051 | 3.95 | 1.2E−5 |
| | (SEQ ID NO: 8) | y₉-y₁₂ | | | | |
| HSP72 | EIAEAYLGGK (I) | y₃-y₈ | 0.27 | 0.39 | 1.21 | 0.11 |
| | (SEQ ID NO: 9) | | | | | |
| HSP7C | GTLDPVEK (I) | y₃-y₇ | 7.72 | 14.9 | 1.93 | 2.2E−6 |
| | (SEQ ID NO: 10) | | | | | |
| HSP74 | AFSDPFVEAEK (III) | y₄, y₆-y₉ | 0.73 | 0.89 | 1.23 | 1.3E−3 |
| | (SEQ ID NO: 11) | | | | | |
| GRP78 | VEIIANDQGNR (I) | y₄-y₉ | 48.8 | 110 | 2.25 | 2.7E−4 |
| | (SEQ ID NO: 12) | | | | | |

Protein-Based Display for Hypothesis-Driven Targets.

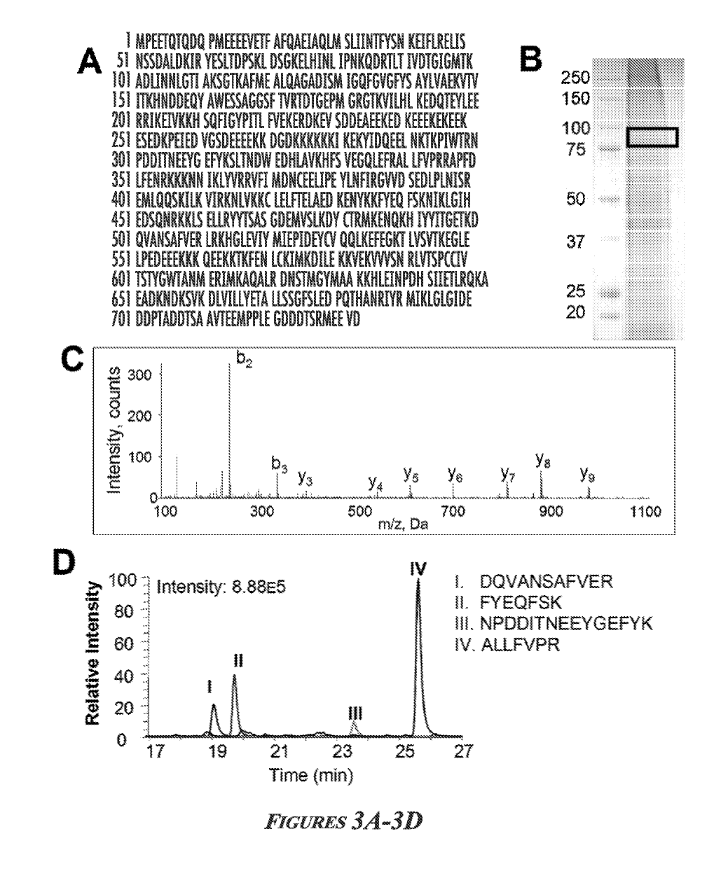

On each protein page, the protein name, UniProt accession number, and sequence were listed. Links were placed in the protein sequence to connect to each webpage that describes a particular peptide that passed the LC-MRM screening criteria; these links were highlighted in yellow when a quantitative assay was developed. Sites of post-translational modification are highlighted in green, when assays for these molecular changes were under development. Selected items from the protein display are shown in FIG. 3, which contains the sequence, SDS-PAGE gel image, LCMS/MS data, and LC-MRM screen for HSP90α. A description of the methods used for protein separation was provided with an image of the results; currently, most of these illustrations were from SDS-PAGE (as in FIG. 3B), but maps using monolithic reversed-phase chromatography for one-dimensional protein separation were also being generated. When available, LC-MS/MS data from previous analyses are included for each target peptide (FIG. 3C) in annotated raw data or Scaffold displays (proteomesoftware.com). Finally, the LC-MRM screens, as shown in FIG. 3D, illustrated all peptides that could be used to quantify the protein in the small amounts of sample injected into the mass spectrometer (0.5-2.5 μg of protein from 5,000 to 25,000 cells fractionated by SDS-PAGE). These peptides should prove to have the most robust ion signals with little interference from other molecules. For HSP90α, four peptides could be detected in the initial screen: NPDDITNEEYGEFYK (SEQ ID NO:13), ALLFVPR (SEQ ID NO:3), FYEQFSK (SEQ ID NO:14), and DQVANSAFVER (SEQ ID NO:15) (assays have been developed for the underlined peptides).

Peptide-Based Display for Developed Assays.

In all LC-MRM screens, successfully detected peptides were ranked by the intensity of the sum of all detected transitions and the amount of interference observed in the ion chromatograms. Using this information, the highest intensity peptides were selected as candidate internal standards, which were then synthesized with stable-isotope labels.

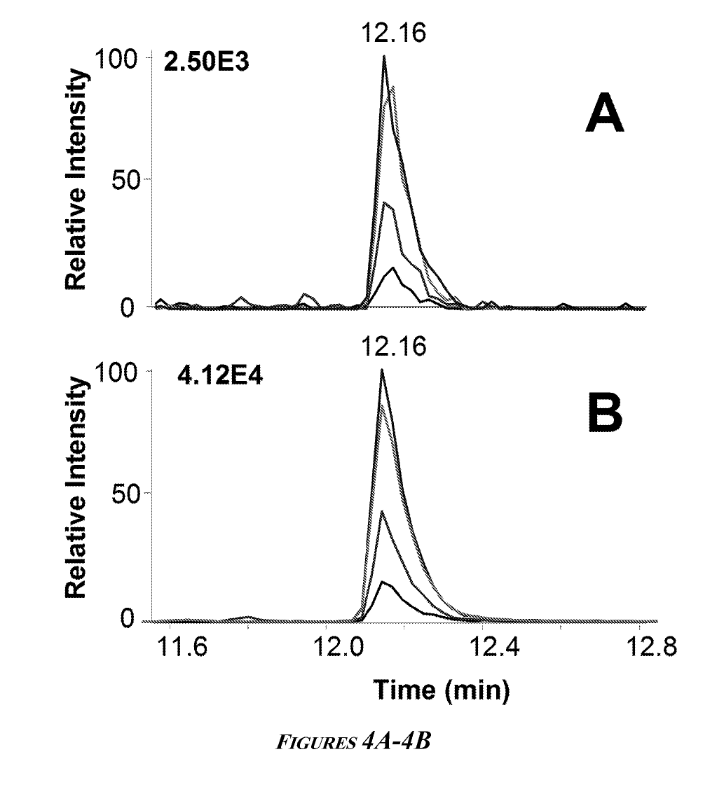

On each peptide webpage, the sequence, location in the protein (noted by amino acid residue numbers), peptide m/z values, isoelectric points, and tables of fragment ions were presented for the native and labeled synthetic sequences. Further characterization of the standard peptide is presented in the peptide synthesis report, which shows the results of purification with reversed-phase HPLC, purity assessment with MALDI-MS, and sequence verification with MS/MS. Each synthetic standard is then analyzed on the triple quadrupole mass spectrometer for assay development, which includes the following steps: MS' to identify the most prominent charge state(s), full-scan MS/MS, optimization of the y ion transitions used to screen the peptide, and optimization of any additional high-intensity and high-specificity (typically high-m/z) transitions. Calibration curves were then created to assess the limit of detection and limit of quantification in order to define the sensitivity of the assay. Finally, the verification of each assay in a cell line model was displayed on the peptide page, as shown by the example in FIG. 4, for HSP90α. Five peptides for HSP90α passed the initial LC-MRM screen: NPDDITNEEYGEFYK (SEQ ID NO:13), LGIHEDSQNR (SEQ ID NO:16), DQVANSAFVER (SEQ ID NO:15), ALLFVPR (SEQ ID NO:3), and FYEQFSK (SEQ ID NO:14). Of these, ALLFVPR (SEQ ID NO:3) and DQVANSAFVER (SEQ ID NO:15) were selected for synthesis due to their intensities (stable-isotope-labeled amino acids are underlined in bold text). After the synthesis of the peptide standard is complete, the assay was tested in a biological sample derived from the cell line used for assay development. To verify the identity of the ion signal in the screen, LC-MRM data must match the retention time of the endogenous peptide detected during screening to the synthetic standard within 0.05 minutes (FIG. 4); plots of detected transitions were created to compare the fragmentation patterns of the synthetic standard and the biological peptide further increasing confidence in its assignment (FIG. 4). The transition ratios, expressed as a percentage of the base peak, should agree within 5%. In the example shown in FIG. 4, all ratios for the endogenous peptide were within 2.5% of the values observed for the synthetic stable-isotope-labeled standard. The co-elution of the biological peptide and internal standard as well as similar fragmentation patterns verify the accuracy of each assay and serve as a checkpoint before any further assay development was performed, including calibration curves and amino acid analysis of the synthetic peptide.

Comparison with ELISA for Absolute Quantification.

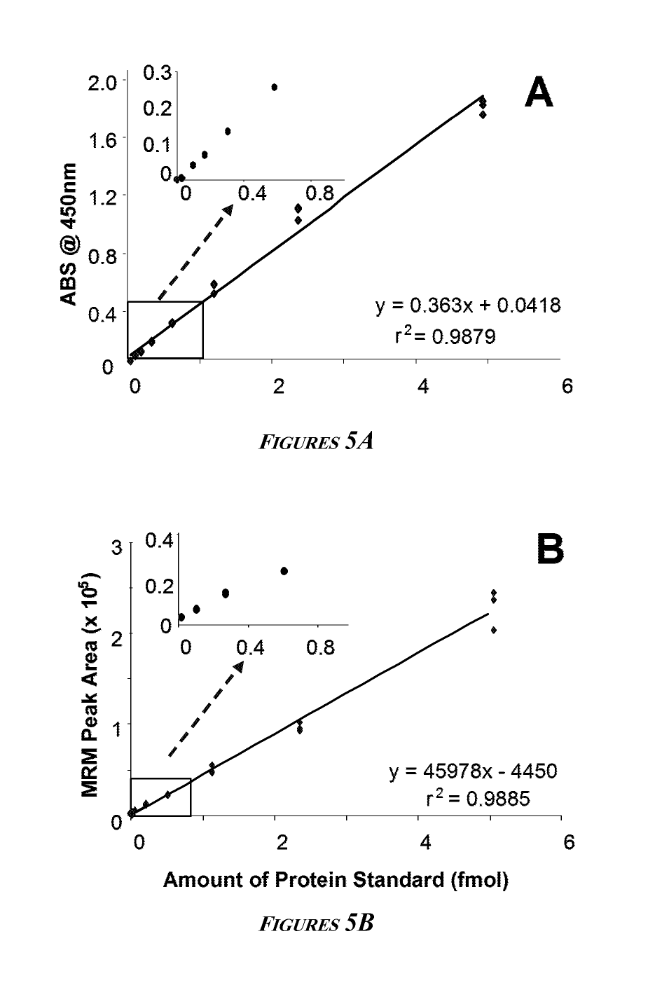

In addition to providing an example of absolute quantification, LC-MRM was compared to an existing method for protein quantification (ELISA). Calibration curves were created using serial dilutions of protein standard using both ELISA measurements of standard protein (FIG. 5A) and LC-MRM monitoring of the ALLFVPR (SEQ ID NO:3) peptide (FIG. 5B). The concentration of the protein standard was determined using the concentration of the heavy-labeled peptide internal standard as calculated by amino acid analysis; the amount determined was higher than the specified value by a factor of 1.9. Equations and r² values were provided for best fit lines on both plots to enable comparison and illustrate the fact that similar linear responses could be obtained for both methods across this range of protein amounts. HSP90α was then measured in 500-50,000 multiple myeloma cells from the RPMI-8226 line using both techniques (FIG. 5C). The absolute quantification by ELISA was based on the measurements of standard protein, while LC-MRM data were quantified by the use of an internal stable-isotope-labeled peptide standard. Using lysates from 50,000 cells, the amounts detected by ELISA, 14.9±0.7 fmol, and the value obtained by LC-MRM, 12.0±0.2 fmol, were systematically offset by a factor of 0.775 (FIG. 5D). The difference could be due to incomplete digestion and peptide recovery prior to LC-MRM analysis. The limits of detection and quantification were slightly better for LC-MRM when compared with ELISA. The median CV values for both sets of measurements were similar: 5.9% for LC-MRM and 10.4% for ELISA. The variability in both methods increased with decreasing sample amount: for example, the values ranged from 1.5% for 50,000 cells to 17% for 500 cells in LC-MRM. The highest CV values observed for ELISA were 22% when analyzing amounts between 500 to 2,500 cells. Using this example for illustration, the LC-MRM methods and reagents described in the QuAD could provide utility for absolute quantification that compared well with antibody-based techniques in amounts of cells expected from patient samples (e.g. bone marrow aspirates).

Implementation in Relative and Absolute Quantification of HSPs in Multiple Myeloma Cells and their Modulation by 17-DMAG Treatment.

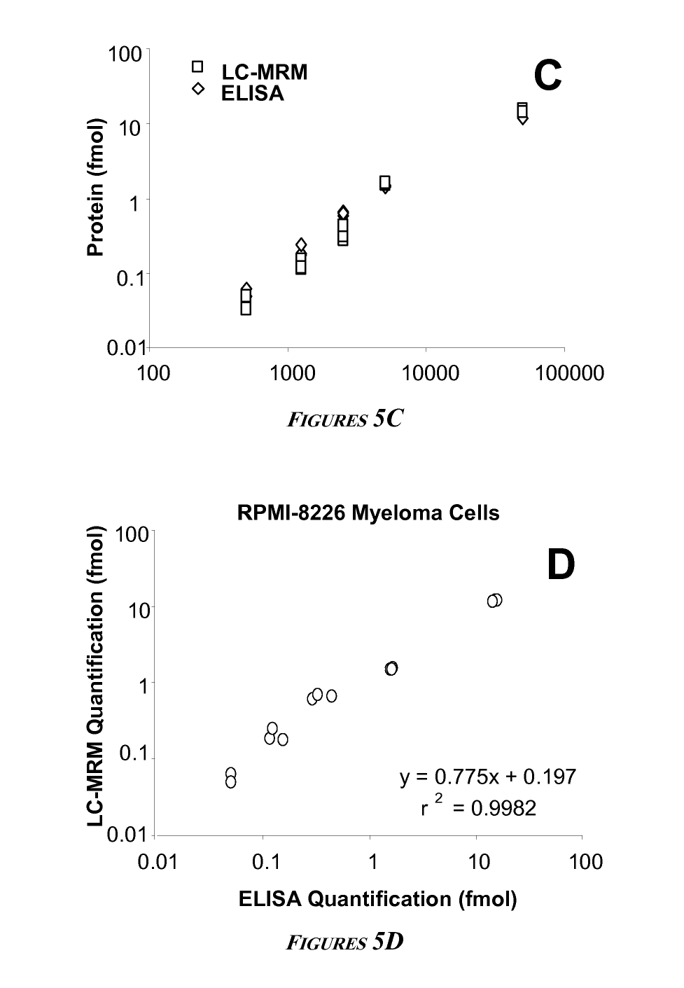

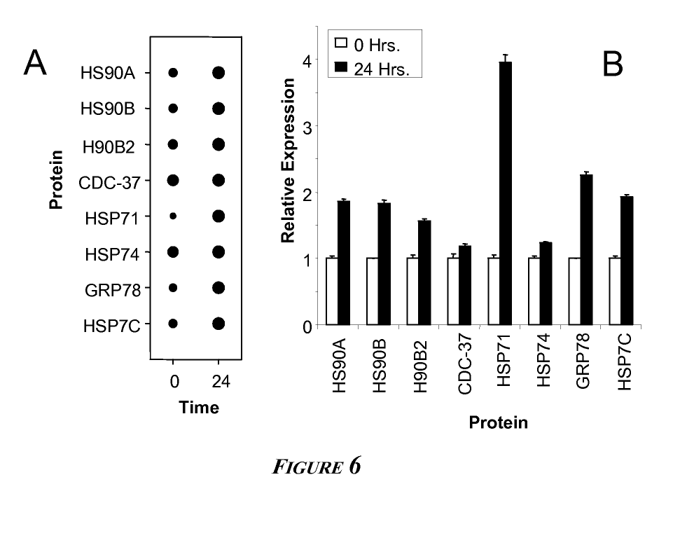

To illustrate the utility of these assays in monitoring response to treatment, the expression of selected HSPs was measured before and 24 hours after treatment of RPMI-8226 multiple myeloma cells with 17-DMAG. Although the LC-MRM screens for assay development were carried out using SDS-PAGE for protein fractionation, these endogenous proteins are expressed at sufficient levels that they can be monitored in tryptic digests of whole cell lysate, enabling higher throughput analysis. The endogenous and standard peptides monitored in each assay, the data for absolute quantification, and the changes in expression with treatment are reported in Table 1; p values were calculated using Student's t-tests. Relative expression is also shown in FIG. 6A using plots analogous to “dot” immunoblots or reversed-phase protein arrays (Zhang et al., Bioinformatics 25:650-4 (2009); Park et al., Mol. Cancer Ther. 9:257-67 (2010)), which enable rapid detection of the most significant changes. The mean and standard deviation of each measurement was plotted in bar graph format in FIG. 6B to examine the assay performance as well as the fold change in expression. Significant upregulation (p<0.01) is noted for several HSPs, including HSP90α and HSP90β as well as HSP71. Changes in the expression of HSP90α and HSP90β were not visualized in traditional Western blotting analysis of HSP expression in RPMI-8226 cells treated with 17-allylamino-17-demethoxygeldanamycin (17-AAG), but could be measured as significantly different using quantitative Western blotting with an Odyssey Infrared Imaging System (Caervantes-Gomez et al., Cancer Res. 69:3947-54 (2009); Stuhmer et al., Leukemia 22:1604-12 (2008)). The precise quantification from LC-MRM enables detection of 1.5- to 1.8-fold changes in protein expression at 24 hours after treatment when compared with controls. Quantifications of increased HSP71 and HSC71 expression, which were among the compensatory mechanisms for HSP90 inhibition, were also possible using LC-MRM, which indicated modulation of expression by factors of 3.95 and 1.93, respectively. The modulations in HSP expression as measured by LC-MRM appear to be in agreement with the prior data acquired by quantitative Western blotting (Caervantes-Gomez et al., Cancer Res. 69:3947-54 (2009)). Additional changes were observed for other chaperones, including GRP78.

To define advantages of this change in technology, the LC-MRM experiments used less than 0.5 μg of protein, whereas Western blots typically use 50-100 μg of protein. Furthermore, the multiplexed analysis of numerous analytes in LC-MRM enables detection of the proteins in a single sample, as opposed to aliquots of the same cell lysate and this set of assays could be further expanded to include other endogenous proteins of interest.

### Example 2

### Evaluation of Direct-Infusion Multiple Reaction Monitoring (DI-MRM) Mass Spectrometry for Quantification of Heat Shock Proteins

Materials and Methods

Reagents.

All chemicals were purchased from Sigma-Aldrich (St. Louis, Mo.) at the highest purity, unless otherwise specified. Formic acid was acquired from Fluka (Sigma-Aldrich). HPLC solvents (water and acetonitrile) were from Burdick and Jackson (Honeywell, Muskegon, Mich.). Bovine serum albumin (BSA) was purchased from Sigma-Aldrich (96% purity).

Synthesis and DI-MRM of Standard Peptides.

Standard peptides were synthesized and characterized as described previously (Remily-Wood et al., Proteomics Clin. Appl. 5:383-96 (2011)). An automated chip-based nanoelectrospray ion source, (NanoMate100, Advion BioSciences, Ithaca, N.Y.), was mounted on a triple quadrupole mass spectrometer (TSQ Vantage, Thermo, San Jose, Calif.). To evaluate the effects of experimental conditions and instrument parameters on the observed peptide fragmentation pattern, two synthetic peptides, ALLFVPR (SEQ ID NO:3) (containing stable isotope-labeled proline) and IEADSESQEEIIR (SEQ ID NO:17) were diluted to 1 μM in 50% aqueous ACN with 0.1% formic acid. For synthetic peptide infusion, the spray voltage was tuned between 1.5 and 1.9 kV for stable ion signal. The gas pressure was set to 0.3 psi. Data were acquired at 20 ms per transition for 2 minutes using m/z 0.002 scan widths. The quadrupole resolution values were modulated from 0.2 to 0.7.

Standard Protein DI-MRM.

As described below, a dilution series of BSA digest was created in 50% aqueous acetonitrile with 0.1% formic acid spiked with five synthetic peptide standards (20 nM). In DI-MRM, the spray voltage was set to 1.6 kV, and the gas pressure was set to 0.3 psi. Peptide selection was performed using Q1=0.4; fragment ion selection was performed using Q3=0.7. Transitions (n=36) were monitored for 20 ms each with scan width set to 0.002; data were acquired for 5 minutes. Relative and absolute quantification values were calculated using the ratio of the endogenous and standard peptide ion signals observed for all monitored transitions.

In-Solution Digestion of BSA Standard Protein.

To prepare for in-solution digestion, aliquots of BSA were denatured using 8M urea in 100 mM ammonium bicarbonate, reduced with 4 mM tris-carboxyethylphosphine (TCEP) at 50° C., for 15 minutes), and then alkylated with 20 mM iodoacetamide at room temperature for 30 minutes in the dark. Before tryptic digestion, 100 mM ammonium bicarbonate buffer was added to reduce the urea concentration to 1 M. Modified trypsin (Promega, Madison, Wis.) was added at an enzyme-to-substrate ratio of 1:50 (w/w); each digestion was incubated at 37° C. overnight. Peptides were extracted with pipette-tip columns (μC18 ZipTips, Millipore, Billerica, Mass.). After concentration by vacuum centrifugation (Speedvac, Thermo), peptides were resuspended in aqueous 2% acetonitrile with 0.1% formic acid.

Cell Culture, Lysis, and HSP Sample Preparation for Mass Spectrometry.

RPMI-8226 and 8226/LR5 cells were maintained as previously described (Bellamy et al., Cancer Res. 51:995-1002 (1991); Remily-Wood et al., Proteomics Clin. Appl. 5:383-96 (2011)). The heat shock protein 90 inhibitor, 17-desmethoxy-17-N,N-dimethylaminoethylamino-geldanamycin (17-DMAG), was dissolved in DMSO (100 μM stock). Based on prior IC₅₀ measurements, the RPMI-8226 cells were treated with 100 nM 17-DMAG for 24 hours, and 8226/LR5 cells were treated with 50 nM 17-DMAG. Cells (n=10⁶) were lysed using in aqueous 100 mM ammonium bicarbonate containing 8M urea for protein denaturation. Protein concentrations were measured by Bradford assays; consistent aliquots of total protein (380 ng) were injected for LC-MRM, and the same solutions were used for DI-MRM. Digestion, addition of SIS peptides, and mass spectrometry sample preparation were performed as described for standard proteins.

LC-MRM Quantification.

For HSP proteins in cell lysate, 184 transitions from 32 peptides (see Table 2) were monitored as previously described (Remily-Wood et al., Proteomics Clin. Appl. 5:383-96 (2011)). Both relative and absolute quantification were calculated from the sum of the peak areas for all detected transitions using Skyline (MacLean et al., Bioinformatics 26:966-8 (2010)) or the combination of MRMer (Martin et al., Mol. Cell Proteomics 7:2270-8 (2008)) and Post-MRMer (http://proteome.moffitt.org).

Calculation of Correction Factors to Account for Interference in DI-MRM.

Additional peaks observed in LC-MRM would constitute interference in DI-MRM for transitions from either endogenous peptides or SIS peptides. To evaluate the ability to eliminate these contributions, correction factors were calculated based on LC-MRM data using the following set of equations (Equations 1 and 2):

$$1. Correction Factor ( CF ) = ( LC \text{-} MRM Peptide PA ) / ( TIC PA )$$

$$2. Corrected DI \text{-} MRM Ratio = \frac{( DI \text{-} MRM PAendogenous ) \times CFendogenous}{( DI \text{-} MRM PAstandard ) \times CFstandard}$$

where PA is peak area and TIC is total ion chromatogram (i.e. the total signal observed for that transition throughout the entire LC separation). This correction factor strategy may not be applicable to samples that could have different biological backgrounds (e.g. different cell types or tissues) and therefore different interference contributions.

TABLE 2
LC-MRM and DI-MRM Assays Developed for Measuring the Expression
of Heat Shock Proteins. For each protein, the Uniprot accession,
peptide(s), label, and transitions for LC-MRM analysis are listed.
Transitions for DI-MRM were selected using a cutoff value of 40%
for interference contribution from other ion signals observed in
that transition during LC-MRM. GAPDH was used as a control for
evaluation of protein loading.

| Protein | | | LC-MRM | DI-MRM |
|---|---|---|---|---|
| (Uniprot) | Peptide | Label | Transitions | Transitions |
| HS90A | ALLFVPR | P₆: ¹³C₅, ¹⁵N | y₃-y₆ | y₃-y₅ |
| | (SEQ ID NO: 3) | | | |
| | EQVANSAFVER | V₉: ¹³C₅, ¹⁵N | y₄-y₉ | y₅, y₆, |
| | (SEQ ID NO: 18) | | | y₈, y₉ |
| HS90B | ALLFIPR | P₆: ¹³C₅, ¹⁵N | y₃-y₆ | y₃-y₅ |
| | (SEQ ID NO: 4) | | | |
| | DQVANSAFVER | V₉: ¹³C₅, ¹⁵N | y₄-y₉ | y₅-y₉ |
| | (SEQ ID NO: 15) | | | |
| CDC37 | LQAEAQQLR | L₈: ¹³C₆, ¹⁵N | y₄-y₇ | y₇ |
| | (SEQ ID NO: 6) | | | |
| | EGEEAGPGDPLLEAVPK | P₁₆: ¹³C₅, ¹⁵N | y₄, y₅, y₈, | y₈, |
| | (SEQ ID NO: 19) | | y₉, y₁₁-y₁₃ | y₁₁-y₁₃ |
| HSP71 | NQVALNPQNTVFDAK | V₁₁: ¹³C₅, ¹⁵N | y₃, y₄, | y₃, y₄, |
| | (SEQ ID NO: 8) | | y₉-y₁₂ | y₉-y₁₂ |
| HSP7C | GTLDPVEK | V₆: ¹³C₅, ¹⁵N | y₃-y₇ | y₄, y₆ |
| | (SEQ ID NO: 10) | | | |
| HSP74 | AFSDPFVEAEK | V₇: ¹³C₅, ¹⁵N | y₄, y₆-y₉ | y₇, y₉ |
| | (SEQ ID NO: 11) | | | |
| HSPA5 | VEIIANDQGNR | V₁: ¹³C₅, ¹⁵N | y₄-y₉ | y₇, y₉ |
| | (SEQ ID NO: 12) | | | |
| HSP7E | FTVLFPSGTPLPAR | P₁₂: ¹³C₅, ¹⁵N | y₃, y₅, | y₉-y₁₁ |
| | (SEQ ID NO: 20) | | y₇-y₁₁ | |
| GAPDH | VGVNGFGR | G₇ → A | y₃-y₇ | y₃-y₇ |
| | (SEQ ID NO: 21) | | | |

Results

Stability of Peptide Transition Ratios Under Different Conditions.

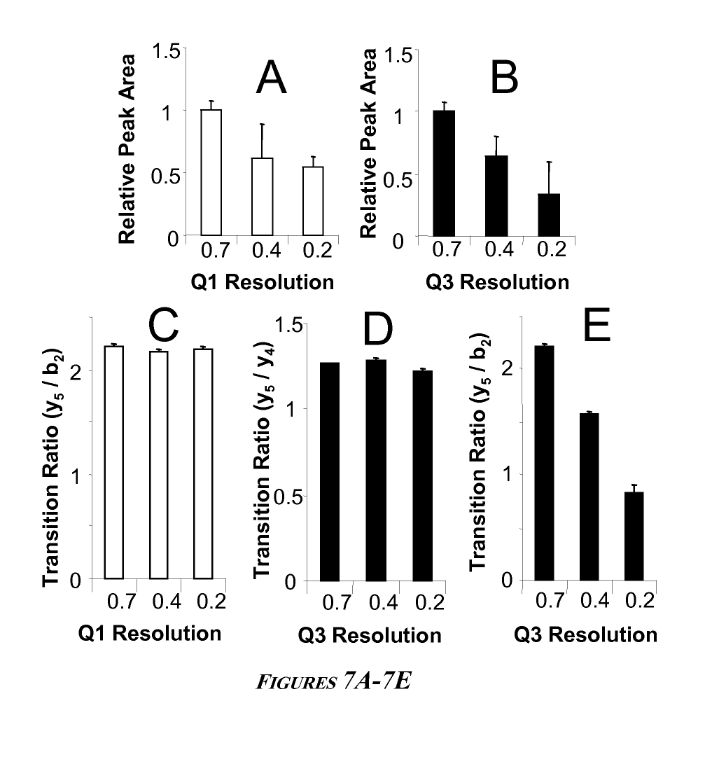

In DI-MRM, verification of the identity of the target peptide can only be achieved using the ratios of the fragment ion intensities. For that reason, the effects of different parameters on the transition ratios were investigated. The effects of quadrupole resolution settings on fragment ion transmission and transition ratios were investigated using two synthetic peptides: ALLFVPR (SEQ ID NO:3) (containing labeled proline) and IEADSESQEEIIR (SEQ ID NO:17) (FIG. 7). Decreasing values for Q1 and Q3 resolution led to progressively lower amounts of signal. Q1 resolution did not have an effect on fragment ion ratios, but modulation of Q3 did.

The changes in transition ratios due to Q3 resolution were most notable when comparing fragment ions with low m/z values (<300) to those with higher m/z values (>500). Therefore, Q1 and Q3 settings should not be varied during an experiment. If narrower values were selected for Q3 resolution, the transition ratios must be re-evaluated. Variations in the solvent system (from 10% to 90% ACN) did not have any effect on fragmentation patterns. To enable comparison with LC-MRM, 50% aqueous acetonitrile with 0.1% formic acid was selected as the solvent system for DI-MRM. Data were acquired for the BSA digest (50 nM) at 7 different spray voltages (from 1.5 to 2.0 kV). Loss of low intensity ion signals was noted at the two lowest spray voltages, but transitions with sufficient intensity (above 20 a.u.) did not show significant differences in fragmentation patterns (transition ratio CV values <2). In order to eliminate ion signal loss, higher spray voltages were selected for the rest of the experiments (1.6 to 1.9 kV for standard peptides and protein digests and 1.9 kV for digested whole cell lysate).

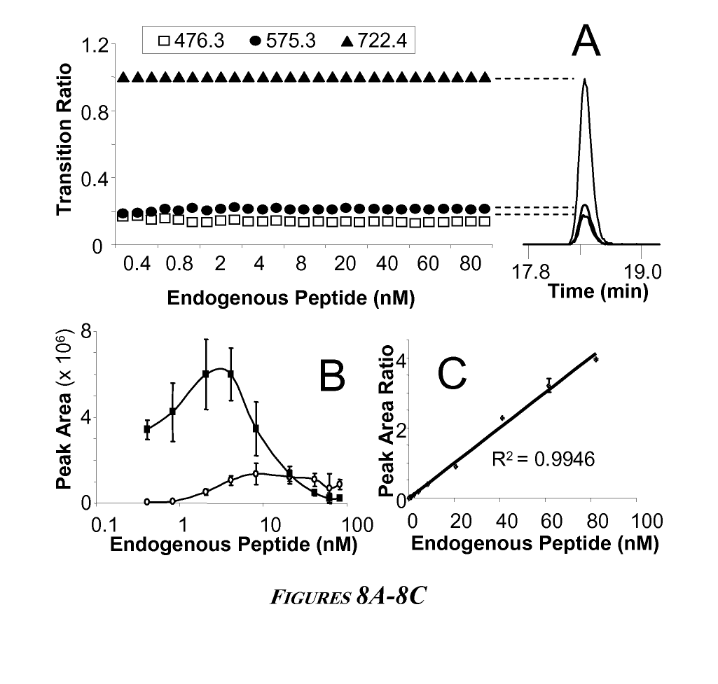

Nine different concentrations (from 0.4 to 80 nM) of the BSA digest were prepared to evaluate DI-MRM reproducibility, sensitivity and linearity of response. Prior to sample analysis, the solvent blank was analyzed as a control. Among the 36 transitions monitored for endogenous and spiked standard BSA peptides, most intensity values were less than 1 a.u. with the maximum observed value ˜3 a.u. For peptides with the lowest amounts of ion signal, DDSPSLPK (SEQ ID NO:22) and QTALVELLK (SEQ ID NO:23), the CV values for transition ratios were below 10% for all samples with concentrations greater than or equal to 2 nM. For the peptides with the highest ion signals (peak intensity >300 a.u.), AEFVEVTK (SEQ ID NO:24) and YLYEIAR (SEQ ID NO:25), the CV values for the transition ratios measured across the entire dilution series were less than 7%; for concentrations >2 nM, the CV values of the transition ratios were less than 2%, indicating the high degree of reproducibility for the transition ratios in DI-MRM. DI-MRM sensitivity was established using consistency of the transition ratios. The fragmentation pattern of the AEFVEVTK (SEQ ID NO:24) peptide in DI-MRM data was consistent with LC-MRM for all concentrations except 0.4 nM, indicating the threshold for transition ratio verification, and thus for quantification of the peptide (FIG. 8A). At high concentrations of endogenous peptides, saturation effects were notable (FIG. 8B), so matched SIS peptides are required for DI-MRM quantification. However, this DI-MRM assay has high linearity (R²=0.9946) over this range of concentrations (FIG. 8C).

Quantification of Heat Shock Proteins in Digests of Whole Cell Lysates.

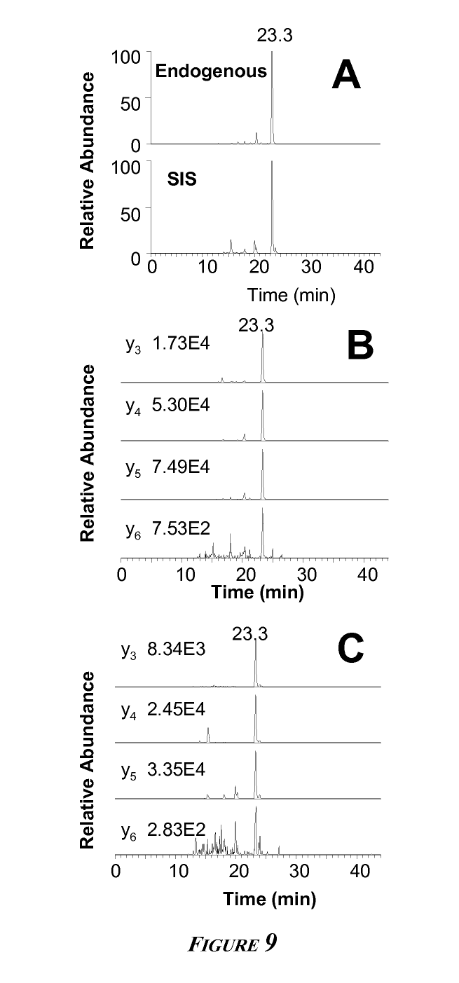

DI-MRM assay development was illustrated using the endogenous and SIS peptides for ALLFVPR (SEQ ID NO:3) from HSP90α (FIG. 9). Total ion signal and potential interferences were evaluated for each peptide and each transition. A cutoff value of 40% interference was selected to enable evaluation of transitions with little interference as well as the utility of correction factors to reduce or eliminate the contribution of noise and interfering peptide peaks. The level of interference in each transition is included in Table 3, and a heat map of the error induced in relative and absolute quantification plotted against interferences (in %) in both endogenous and standard transitions is shown in FIG. 10.

TABLE 3
Levels of Potential DI-MRM Interferences
Detected in LC-MRM. The total ion chroma-
tograms for each transition were integrated
to explore the amount interference that
would be incorporated into the DI-MRM
measurement. Interference values include
both baseline noise integration and the
contribution of other observed peaks.

| | | Fragment | Inter- |
|---|---|---|---|
| | | m/z | ference |
| Protein | Peptide | Values | (%) |
| CDC37 | Endogenous | 414.3 | 84.57 |
| | EGEEAGPGDPLLEAVPK | 543.3 | 61.65 |
| | (SEQ ID NO: 19) | 866.5 | 10.59 |
| | | 981.6 | 51.75 |
| | | 1135.6 | 14.56 |
| | | 1192.7 | 11.46 |
| | | 1263.7 | 0.00 |
| | SIS | 420.3 | 45.44 |
| | EGEEAGPGDPLLEAVPK | 549.3 | 9.56 |
| | (SEQ ID NO: 19) | 872.5 | 0.00 |
| | | 987.6 | 42.22 |
| | | 1141.6 | 0.00 |
| | | 1198.7 | 0.00 |
| | | 1269.7 | 21.03 |
| CDC 37 | Endogenous | 544.3 | 0.00 |
| | LQAEAQQLR | 615.4 | 43.05 |
| | (SEQ ID NO: 6) | 744.4 | 27.36 |
| | | 815.4 | 9.81 |
| | SIS | 551.3 | 0.00 |
| | LQAEAQQLR | 622.4 | 0.00 |
| | (SEQ ID NO: 6) | 751.4 | 0.00 |
| | | 822.5 | 0.79 |
| HSP71 | Endogenous | 333.2 | 51.59 |
| | NQVALNPQNTVFDAK | 480.2 | 18.63 |
| | (SEQ ID NO: 8) | 1019.5 | 0.72 |
| | | 1133.6 | 6.09 |
| | | 1246.6 | 0.00 |
| | | 1317.7 | 0.00 |
| | SIS | 333.2 | 0.00 |
| | NQVALNPQNTVFDAK | 480.2 | 0.00 |
| | (SEQ ID NO: 8) | 1025.5 | 0.00 |
| | | 1139.6 | 0.00 |
| | | 1252.7 | 0.00 |
| | | 1323.7 | 0.00 |
| HSP74 | Endogenous | 476.2 | 53.72 |
| | AFSDPFVEAEK | 722.4 | 49.11 |
| | (SEQ ID NO: 11) | 819.4 | 9.46 |
| | | 934.5 | 26.07 |
| | | 1021.5 | 6.26 |
| | SIS | 476.2 | 0.00 |
| | AFSDPFVEAEK | 728.4 | 0.00 |
| | (SEQ ID NO: 11) | 825.4 | 4.47 |
| | | 940.5 | 0.00 |
| | | 1027.5 | 0.00 |
| HSP90α | Endogenous | 550.3 | 0.00 |
| | EQVANSAFVER | 621.3 | 0.00 |
| | (SEQ ID NO: 18) | 708.4 | 0.00 |
| | | 822.4 | 0.00 |
| | | 893.4 | 15.69 |
| | | 992.5 | 0.00 |
| | SIS | 556.3 | 61.48 |
| | EQVANSAFVER | 627.3 | 6.87 |
| | (SEQ ID NO: 18) | 714.4 | 9.50 |
| | | 828.4 | 46.81 |
| | | 899.5 | 11.57 |
| | | 998.5 | 1.50 |
| HSP90α | Endogenous | 371.2 | 0.00 |
| | ALLFVPR | 518.3 | 12.32 |
| | (SEQ ID NO: 3) | 631.4 | 15.22 |
| | | 744.5 | 0.00 |
| | SIS | 377.3 | 0.00 |
| | ALLFVPR | 524.3 | 23.74 |
| | (SEQ ID NO: 3) | 637.4 | 30.79 |
| | | 750.5 | 0.00 |
| HSP90β | Endogenous | 385.3 | 0.00 |
| | ALLFIPR | 532.3 | 0.00 |
| | (SEQ ID NO: 4) | 645.4 | 0.00 |
| | | 758.5 | 0.00 |
| | ALLFIPR | 391.3 | 0.00 |
| | (SEQ ID NO: 4) | 538.3 | 0.00 |
| | | 651.4 | 6.26 |
| | | 764.5 | 0.00 |
| HSP90β | Endogenous | 550.3 | 0.00 |
| | DQVANSAFVER | 621.3 | 0.00 |
| | (SEQ ID NO: 15) | 708.4 | 0.00 |
| | | 822.4 | 0.00 |
| | | 893.4 | 0.00 |
| | | 992.5 | 0.00 |
| | SIS | 556.3 | 26.23 |
| | DQVANSAFVER | 627.3 | 8.85 |
| | (SEQ ID NO: 15) | 714.4 | 18.69 |
| | | 828.4 | 13.71 |
| | | 899.5 | 9.35 |
| | | 998.5 | 3.99 |
| HSPA5 | Endogenous | 474.2 | 0.00 |
| | VEIIANDQGNR | 589.3 | 0.00 |
| | (SEQ ID NO: 12) | 703.3 | 0.00 |
| | | 774.3 | 0.00 |
| | | 887.4 | 0.00 |
| | | 1000.5 | 5.56 |
| | SIS | 474.2 | 20.78 |
| | VEIIANDQGNR | 589.3 | 72.99 |
| | (SEQ ID NO: 12) | 703.3 | 37.67 |
| | | 774.3 | 19.23 |
| | | 887.4 | 45.06 |
| | | 1000.5 | 2.58 |
| HSP7E | Endogenous | 343.2 | 37.61 |
| | FTVLFPSGTPLPAR | 553.3 | 73.21 |
| | (SEQ ID NO: 20) | 711.4 | 81.22 |
| | | 798.4 | 40.61 |
| | | 895.5 | 6.09 |
| | | 1042.6 | 28.67 |
| | | 1155.7 | 0.00 |
| | SIS | 349.2 | 0.00 |
| | FTVLFPSGTPLPAR | 559.4 | 0.00 |
| | (SEQ ID NO: 20) | 717.4 | 0.00 |
| | | 804.5 | 0.00 |
| | | 901.5 | 0.00 |
| | | 1048.6 | 0.00 |
| | | 1161.7 | 0.00 |
| HSP7C | Endogenous | 375.2 | 66.67 |
| | GTLDPVEK | 472.3 | 13.56 |
| | (SEQ ID NO: 10) | 587.3 | 52.38 |
| | | 700.4 | 7.38 |
| | | 801.4 | 0.00 |
| | SIS | 381.2 | 0.00 |
| | GTLDPVEK | 478.3 | 4.55 |
| | (SEQ ID NO: 10) | 593.3 | 0.00 |
| | | 706.4 | 0.24 |
| | | 807.4 | 0.00 |

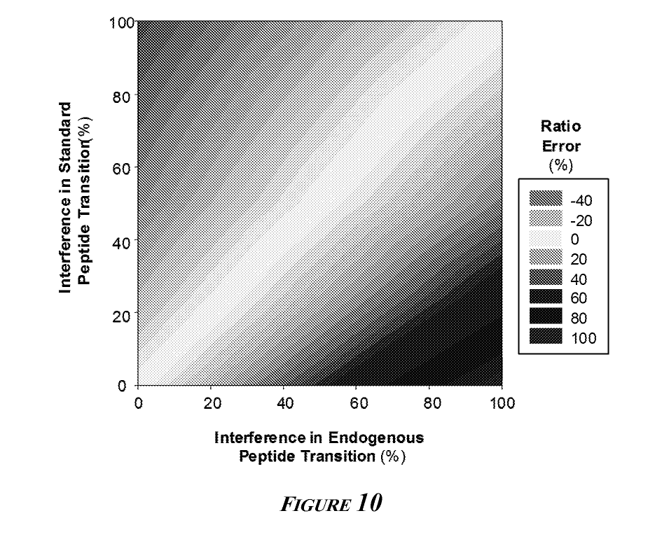

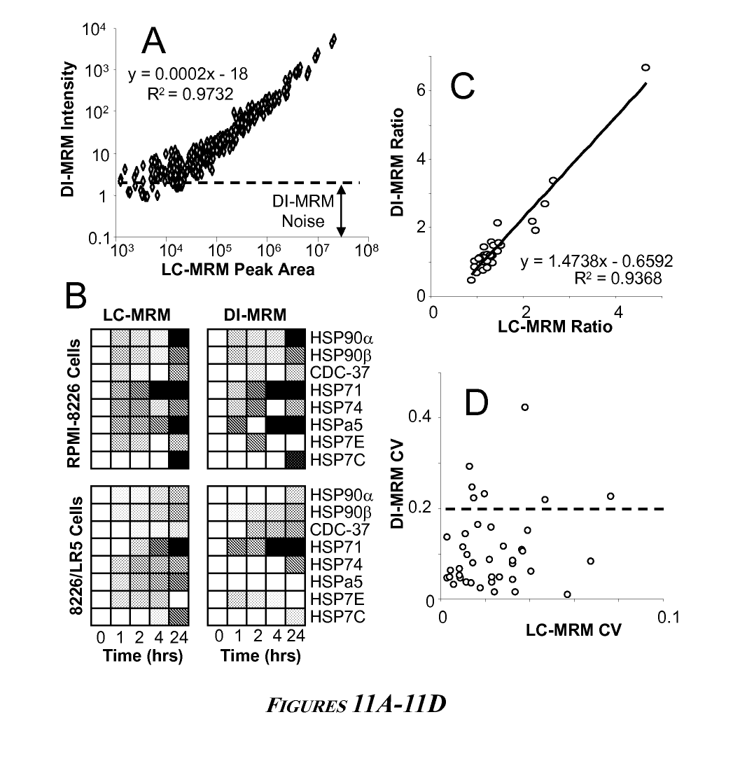

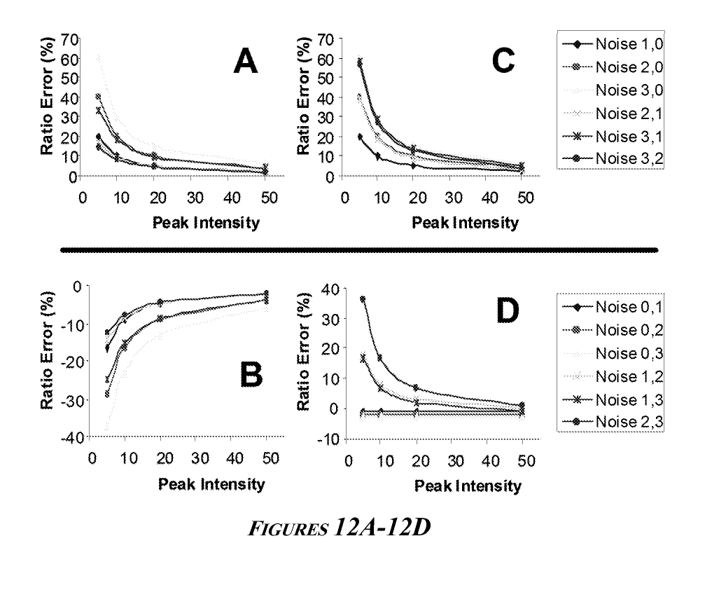

The next step in the development of the DI-MRM assay was to evaluate the amount of ion signal observed for each transition and assess whether it was sufficient for quantification. First, a solvent blank was analyzed to observe the level of background noise in each of the 184 monitored transitions; the maximum value for peak intensity was ˜3 a.u. Then, DI-MRM peak intensity was plotted against LC-MRM peak area (FIG. 11A). Noise contribution can be significant for peptide ion signals at or below 10 a.u. in DI-MRM intensity. A trend line was fitted to the data, which indicated a strong correlation between the two methods (R²=0.9732) when DI-MRM intensity values were greater than 10 a.u. For the peaks with low intensity in DI-MRM (<10 a.u.), the correlation of DI-MRM intensity with LC-MRM peak area was poor (R²=0.2796). Because of the noise contribution, quantification was not reliable for peaks below a certain intensity threshold; theoretical calculations for the effect of baseline noise on the relative and absolute quantification derived from DI-MRM data are shown in FIG. 12. For comparison, the peak intensity values for each peptide (given as the sum of all transitions) are shown in Table 4. Each peak must be evaluated, because the limits of detection and quantification as well as the accuracy of the values will depend on the amount of background noise observed in each transition.

TABLE 4
Peak Intensity Values for Endogenous HSP
Peptides. Replicate measurements for 2
minutes were used to evaluate the average
peak intensity of the sum of all transitions.
Based on the data from FIG. 7, the desired
amount of endogenous signal would be at
least 100 a.u. Therefore, some of these
measurements that have lower values for
peak intensity would be expected to have
the potential for error in relative and
absolute quantification.

| | | Replicate Measurements | | | | |
|---|---|---|---|---|---|---|
| Pro- | | (2 minutes each) | | | | |
| tein | Peptide | A | B | C | D | E |
| CDC37 | EGEEAGPGDPL | 17.8 | 24.7 | 24.6 | 29.1 | 22.1 |
| | LEAVPK | | | | | |
| | (SEQ ID | | | | | |
| | NO: 19) | | | | | |
| | LQAEAQQLR | 41.8 | 44.2 | 36.5 | 44 | 42.7 |
| | (SEQ ID | | | | | |
| | NO: 6) | | | | | |
| HSP71 | NQVALNPQNTV | 40.2 | 49.5 | 46.1 | 38.3 | 56 |
| | FDAK | | | | | |
| | (SEQ ID | | | | | |
| | NO: 8) | | | | | |
| HSP74 | AFSDPFVEAEK | 29.5 | 23.8 | 22.1 | 22.5 | 27.5 |
| | (SEQ ID | | | | | |
| | NO: 11) | | | | | |
| HS90A | EQVANSAFVER | 634.6 | 631.6 | 650.9 | 647.9 | 649.3 |
| | (SEQ ID | | | | | |
| | NO: 18) | | | | | |
| | ALLFVPR | 426.5 | 440.1 | 415.1 | 433 | 445.3 |
| | (SEQ ID | | | | | |
| | NO: 3) | | | | | |
| HS90B | ALLFIPR | 685.9 | 689.5 | 673.7 | 685.1 | 704.5 |
| | (SEQ ID | | | | | |
| | NO: 4) | | | | | |
| | DQVANSAFVER | 288.9 | 292.9 | 306.4 | 319.4 | 273.1 |
| | (SEQ ID | | | | | |
| | NO: 15) | | | | | |
| HSPA5 | VEIIANDQGNR | 403.4 | 477.5 | 478.1 | 490.5 | 477.7 |
| | (SEQ ID | | | | | |
| | NO: 12) | | | | | |
| HSP7E | FTVLFPSGTPL | 26.6 | 26.5 | 27.6 | 30.1 | 20.5 |
| | PAR | | | | | |
| | (SEQ ID | | | | | |
| | NO: 20) | | | | | |
| HSP7C | GTLDPVEK | 238.8 | 219.9 | 222.9 | 251.6 | 254.3 |
| | (SEQ ID | | | | | |
| | NO: 10) | | | | | |
| GAPDH | VGVNGFGR | 173.6 | 161.8 | 168 | 169.9 | 165.8 |
| | (SEQ ID | | | | | |
| | NO: 21) | | | | | |

Selection of DI-MRM Acquisition Times.

After transition evaluation, the total sample analysis time was selected (Equation 3): Total Acquisition Time=(# of transitions)(scan time per transition)(# of observations).

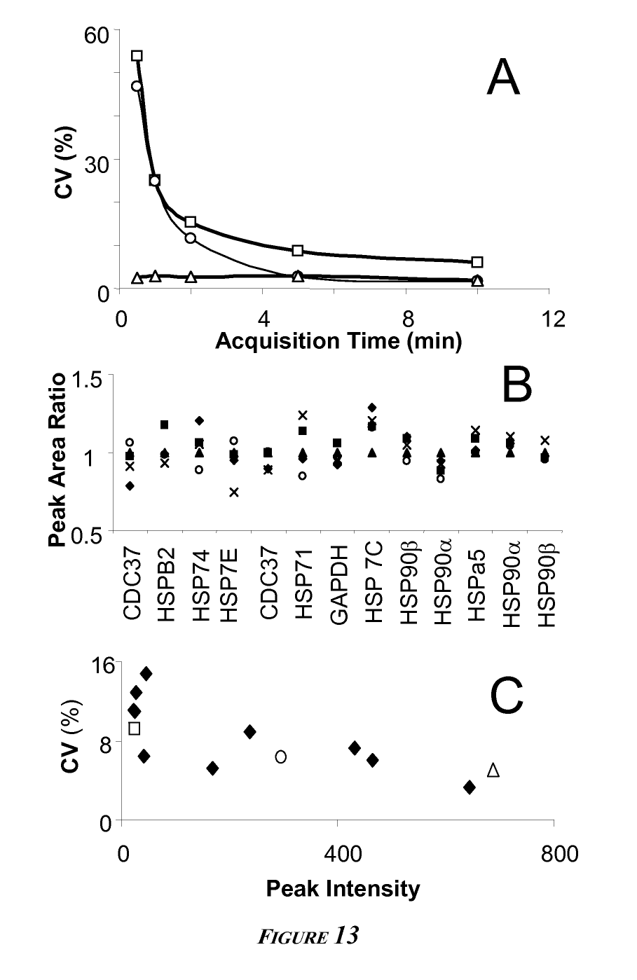

For BSA (monitoring 36 transitions), one minute acquisitions obtained more than 80 observations for each transition and generated reproducible data with CV values typically less than 10%. For analysis of HSP proteins in digests of whole cell lysates, the effect of acquisition time on CV was evaluated using data acquired over spans of 30 seconds to 10 minutes (FIG. 13). Using 20% CV as a maximum cutoff value, data could be acquired in as little as two minutes for this set of transitions (n=184, each sampled 32 times). CV values correlate well with DI-MRM peak intensity; higher intensity values produce more precise measurements. One noteworthy advantage of DI-MRM is that data acquisition time can be lengthened to improve sampling and assay performance, whereas in LC-MRM the peptides can only be detected over their elution profiles. DI-MRM experiments described here typically include >30 observations of each transition. If improved precision was required (e.g. CV<10%), data could be acquired for 5 minutes for HSP monitoring.

Comparison of LC-MRM to DI-MRM Measurements of HSPs in Digests of Whole Cell Lysates.

HSP expression levels were monitored in RPMI-8226 and 8226/LR5 cells following 17-DMAG treatment. In order to examine the trends in protein expression and compare LC-MRM and DI-MRM data sets, heat maps were generated for the relative expression of each protein using ratios to the pretreatment control (FIG. 11B). No proteins decreased significantly, so darker shading indicates increases in protein expression. Overall, the same trends are observed by both LC-MRM and DI-MRM. Most notably, the upregulation of HSP90α, HSP90β, and HSP71 are diminished and delayed in the melphalan-resistant 8226/LR5 myeloma cells, when compared to RPMI-8226 cells. Here, DI-MRM is consistent with LC-MRM in the pattern of changes in protein expression, but the use of DI-MRM for relative and absolute quantification must be further explored. DI-MRM expression ratio measurements of HSPs from RPMI-8226 cell lysates correlate well (R²=0.9368) to the corresponding LC-MRM values (FIG. 11C). The slope of the trend line (1.5) is driven mainly by the differences in ratios with higher fold change values; most DI-MRM ratio values were lower than those from LC-MRM data. Comparison of the CV values for the same data set acquired for RPMI-8226 cells is shown in FIG. 11D, illustrating that DI-MRM variability was higher than LC-MRM in these assays applied to digests of whole cell lysate. Even so, most measurements (33/40) still have CV values below 20%, indicating that DI-MRM can still obtain sufficient precision.

A more detailed examination of relative quantification was performed to evaluate the use of raw DI-MRM data and correction factors. Different methods for relative quantification have been compared for evaluating HSP90α expression at 24 hours after treatment (Table 5). In the LC-MRM data, selection of different sets of transitions (y₃-y₆, y₃-y₅, or just y₃) produced the same results, approximately 2.2-fold increase in RPMI-8226 and 1.2-fold increase in 8226/LR5 cells. For RPMI-8226 cells, none of the DI-MRM measurements was significantly different from LC-MRM (p>0.05). Selection of the single transition (y₃) with the least interference in LC-MRM resulted in the highest CV value (30%) and the most disparate value for the change in relative expression. In 8226/LR5 cells, all values for relative quantification of the change in HSP90α were significantly different from the LC-MRM data (p<0.05). The values for the changes in expression of HSP90α were calculated to be 1.1-fold in DI-MRM versus 1.2-fold in LC-MRM. Again, the use of the single transition produced the most disparate data (0.76-fold change in expression). From both cell lines, selection of the unique transition from ALLFVPR (SEQ ID NO:3) with no background peaks in LC-MRM was not the best strategy for DI-MRM. Correction factors improved the agreement between DI-MRM and LC-MRM, but some differences were still significant.

TABLE 5
Comparison of Different Methods for DI-MRM Data Analysis. HSP90α expression
was measured at 0 and 24 hours after treatment with 17-DMAG using DI-MRM
and LC-MRM to quantify the ALLFVPR (SEQ ID NO: 3) peptide. The results
from selection of different sets of transitions and incorporation of interference
correction were compared in terms of relative quantification (fold change)
and CV values. Student's t tests were used to calculate p values.

| | RPMI-8226 Cells | | | | | 8226/LR5 Cells | | | | |
|---|---|---|---|---|---|---|---|---|---|---|
| | (24 hrs after treatment vs. control) | | | | | (24 hrs after treatment vs. control) | | | | |
| | LC-MRM | | DI-MRM | | | LC-MRM | | DI-MRM | | |
| | Fold | CV | Fold | CV | p | Fold | CV | Fold | CV | p |
| Transitions | Change | (%) | Change | (%) | value | Change | (%) | Change | (%) | value |
| y₃-y₆ | 2.21 | 1.0 | 1.86 | 9.6 | 0.077 | 1.23 | 0.9 | 1.14 | 2.0 | 0.0068 |
| (All LC-MRM | | | | | | | | | | |
| Transitions) | | | | | | | | | | |
| y₃-y₆ | 2.21 | 1.0 | 1.87 | 11 | 0.099 | 1.23 | 0.9 | 1.12 | 0.9 | 0.00023 |
| (DI-MRM | | | | | | | | | | |
| Interference- | | | | | | | | | | |
| Corrected) | | | | | | | | | | |
| y₃-y₅ | 2.21 | 1.0 | 1.85 | 9.6 | 0.076 | 1.23 | 0.9 | 1.13 | 2.0 | 0.0078 |
| (Selected | | | | | | | | | | |
| Transitions) | | | | | | | | | | |
| y₃-y₅ | 2.21 | 1.0 | 1.85 | 9.8 | 0.079 | 1.23 | 0.9 | 1.14 | 1.6 | 0.0037 |
| (DI-MRM | | | | | | | | | | |
| Interference- | | | | | | | | | | |
| Corrected) | | | | | | | | | | |
| y₃ | 2.19 | 3.0 | 2.96 | 30 | 0.27 | 1.26 | 6.7 | 0.76 | 12 | 0.011 |
| (Least | | | | | | | | | | |
| Interference) | | | | | | | | | | |

Based on these results, the use of correction factors to eliminate the contribution of interference in DI-MRM data for other peptides was evaluated. Relative quantification of the other HSPs was calculated using all LC-MRM transitions with correction factors for elimination of the contribution of interference (Table 6); in addition, relative quantification was also calculated using selected LC-MRM transitions (below 40% interference contribution) with interference correction (Table 7). Most of the LC-MRM and DI-MRM data are concordant; those values that show significant differences based on Student's t tests had very high precision in both LC-MRM and DI-MRM (CV values typically less than 2%). Those measurements also still showed the same trends in protein expression, as noted above (FIG. 11). Agreement in the values for relative quantification was noted for the HSP peptides that had the highest ion signals, but deviations were observed in peptides with lower amounts of DI-MRM ion signals. Similar to relative quantification, the interference-corrected values for absolute quantification (Table 8) were reliable for peptides with higher intensity values, but discordant when the peak intensities were low. The expression levels of HSP90α, HSP90β, CDC37, HSP7C, and HSPA5 were consistent by LC-MRM and DI-MRM (p>0.05), but some of the errors in the average values were >20% when comparing against LC-MRM data. DI-MRM measurements of the expression levels of proteins with low intensity peptides, such as HSP71, HSP74, and HSP7E, were significantly different from the LC-MRM results. Therefore, DI-MRM was most appropriate for high intensity peptides (>100 a.u.); however, data for proteins like CDC37 indicate that lower intensity DI-MRM measurements can be consistent with LC-MRM when corrected for the contributions from interference.

TABLE 6
Comparison of Relative Quantification of HSPs using LC-MRM and Interference-Corrected
DI-MRM Data. For each protein measurement, the fold change and CV (%) values are shown
for LC-MRM and DI-MRM. Student's t tests were used to calculate p values.

| | RPMI-8226 Cells | | | | | 8226/LR5 Cells | | | | |
|---|---|---|---|---|---|---|---|---|---|---|
| | (24 hrs after treatment vs. control) | | | | | (24 hrs after treatment vs. control) | | | | |
| Protein | LC-MRM | | DI-MRM | | | LC-MRM | | DI-MRM | | |
| (Uniprot | Fold | CV | Fold | CV | p | Fold | CV | Fold | CV | p |
| Accession) | Change | (%) | Change | (%) | value | Change | (%) | Change | (%) | value |
| HS90A | 2.21 | 1.0 | 1.86 | 10 | 0.089 | 1.23 | 0.9 | 1.12 | 0.9 | 0.00023 |
| HS90B | 1.53 | 1.2 | 1.46 | 6.9 | 0.24 | 1.28 | 0.2 | 1.23 | 2.7 | 0.12 |
| CDC37 | 1.33 | 1.5 | 1.08 | 20 | 0.18 | 1.08 | 3.3 | 1.24 | 12 | 0.22 |
| HSP71 | 4.67 | 2.6 | 6.10 | 4.2 | 0.0031 | 3.54 | 2.0 | 5.50 | 2.3 | 0.032 |
| HSP7C | 2.28 | 1.4 | 1.52 | 3.3 | 0.00021 | 1.56 | 0.3 | 0.94 | 3.3 | 0.00084 |
| HSP74 | 1.45 | 1.7 | 1.22 | 18 | 0.21 | 1.34 | 20 | 1.33 | 38 | 0.99 |
| HSPA5 | 2.66 | 2.2 | 3.43 | 5.5 | 0.022 | 1.53 | 1.9 | 0.86 | 18 | 0.018 |
| HSP7E | 1.23 | 0.3 | 0.87 | 20 | 0.07 | 0.99 | 1.2 | 0.93 | 7.8 | 0.28 |

TABLE 7
Comparison of Relative Quantification of HSPs using LC-MRM and Noise Corrected DI-MRM
Data using only Transitions below 40% Interference Contribution. For each protein measurement,
the fold change and CV (%) values are shown for LC-MRM and DI-MRM. Student's t tests
were used to calculate p values. Data can be compared with Table 8.

| | RPMI-8226 Cells | | | | | 8226/LR5 Cells | | | | |
|---|---|---|---|---|---|---|---|---|---|---|
| | (24 hrs after treatment vs. control) | | | | | (24 hrs after treatment vs. control) | | | | |
| Protein | LC-MRM | | DI-MRM | | | LC-MRM | | DI-MRM | | |
| (Uniprot | Fold | CV | Fold | CV | p | Fold | CV | Fold | CV | p |
| Accession) | Change | (%) | Change | (%) | value | Change | (%) | Change | (%) | value |
| HS90A | 2.21 | 1.0 | 1.85 | 9.8 | 0.079 | 1.23 | 0.9 | 1.14 | 1.6 | 0.0037 |
| HS90B | 1.53 | 1.2 | 1.46 | 4 | 0.19 | 1.28 | 0.2 | 1.23 | 2.6 | 0.11 |
| CDC37 | 1.33 | 1.5 | 1.20 | 22 | 0.47 | 1.08 | 3.3 | 1.35 | 12 | 0.11 |
| HSP71 | 4.67 | 2.6 | 6.10 | 5.0 | 0.0048 | 3.54 | 2.0 | 5.51 | 1.8 | 0.017 |
| HSP7C | 2.28 | 1.4 | 1.84 | 8.4 | 0.0035 | 1.56 | 0.3 | 1.14 | 4.1 | 0.0044 |
| HSP74 | 1.45 | 1.7 | 1.37 | 16 | 0.60 | 1.34 | 20 | 1.41 | 29 | 0.80 |
| HSPA5 | 2.66 | 2.2 | 2.53 | 9.4 | 0.46 | 1.53 | 1.9 | 0.90 | 34 | 0.073 |
| HSP7E | 1.23 | 0.3 | 0.81 | 28 | 0.088 | 0.99 | 1.2 | 1.05 | 18 | 0.66 |

TABLE 8
LC-MRM and DI-MRM Measurements of Baseline HSP Protein Expression Levels in RPMI-
8226 and 8226/LR5 Cells. For each protein measurement, the average amounts and
standard deviations (in femtomoles per microgram of total protein) are shown
for LC-MRM and DI-MRM. Student's t test was used to calculate p values.

| | RPMI-8226 Cells | | | | 8226/LR5 Cells | | | |
|---|---|---|---|---|---|---|---|---|
| | (fmol/μg total protein) | | | | (fmol/μg total protein) | | | |
| | | | Error | p | | | Error | p |
| Protein | LC-MRM | DI-MRM | (%) | value | LC-MRM | DI-MRM | (%) | value |
| HS90A | 110.3 ± 4.5 | 113.4 ± 6.9 | 2.8 | 0.56 | 134.5 ± 2.2 | 151.8 ± 4.8 | 12.9 | 0.011 |
| HS90B | 78.0 ± 0.47 | 77.5 ± 2.5 | −0.6 | 0.75 | 97.9 ± 0.79 | 97.5 ± 2.2 | −0.4 | 0.76 |
| CDC37 | 9.0 ± 0.61 | 9.2 ± 0.76 | 22 | 0.74 | 12.3 ± 0.58 | 10.1 ± 2.5 | −17.9 | 0.44 |
| HSP71 | 128.3 ± 6.01 | 72.1 ± 15.8 | −43.7 | 0.01 | 113.6 ± 5.3 | 37.8 ± 11.3 | −66.7 | 0.0018 |
| HSP7C | 76.2 ± 3.0 | 101.9 ± 15.4 | 33.7 | 0.11 | 77.2 ± 0.6 | 138.1 ± 7.4 | 78.9 | 0.055 |
| HSP74 | 7.16 ± 0.27 | 2.8 ± 1.2 | −60.9 | 0.025 | 5.2 ± 0.4 | 1.82 ± 0.48 | −65 | 0.00059 |
| HSPA5 | 481.6 ± 5.7 | 661.3 ± 71.2 | 37.3 | 0.18 | 432.0 ± 9.0 | 954.2 ± 158.4 | 120.9 | 0.13 |
| HSP7E | 1.92 ± 0.15 | 0.93 ± 0.21 | −51.6 | 0.0026 | 2.61 ± 0.07 | 1.53 ± 0.97 | −41.4 | 0.19 |

### Example 3

### The Heat Shock Protein-90 Inhibitor XL888 Overcomes BRAF Inhibitor Resistance Mediated Through Diverse Mechanisms

Materials and Methods

Cell Culture and Generation of BRAF Inhibitor Resistance.

The parental 1205Lu, WM39 and WM164 melanoma cells lines were genotyped as having the BRAF V600E mutation as described previously (Smalley et al., Br. J. Cancer 96:445-9 (2007)). The M229, M229R, M249 and M249R have been described in (Nazarian et al., Nature 468:973-7 (2010)). The RPMI7951 melanoma cell line was purchased from ATCC. The identities of all cell lines were confirmed by Biosynthesis Inc (Lewisville, Tex.) through STR validation analysis. Naïve and intrinsically resistant lines were cultured in 5% FBS, RPMI. For all studies, all acquired resistant cell lines were maintained in 5% media with the addition of vemurafenib at the following concentrations: 104 for M229R and M249R, 204 for WM164R and 3 μM for 1205LuR.

Growth Inhibition.

Cells were plated at a density of 2.5×10⁴ cells per ml and left to grow overnight before being treated with increasing concentrations of vemurafenib or XL888. After 72 hours, the levels of growth inhibition were examined using the MTT assay (Smalley et al., Br. J. Cancer 96:445-9 (2007)). Data show the mean of at least three independent experiments±the S.E. mean. Vemurafenib and XL888 were dissolved in 100% DMSO and stored at −20° C. as a 10 mM solution.

Western Blotting.

Proteins were extracted and blotted for as described previously (Smalley et al., Br. J. Cancer 96:445-9 (2007)). After analysis, Western blots were stripped once and re-probed for β-actin or GAPDH to demonstrate even protein loading. The antibodies to IGF1R, PDGFRβ, ARAF, CRAF, phospho-AKT Ser473, total AKT, phospho-ERK, total ERK, cyclin D1, phospho-S6, total S6, phospho-BIM (Ser69), total BIM, HSP70 and MCL-1 were from Cell Signaling Technology (Beverly, Mass.). Anti-26S was purchased from Abcam (Cambridge, Mass.), and the antibody against COT was from Santa Cruz Biotechnology (Santa Cruz, Calif.).

Flow Cytometry.

Cells were plated into 6 well tissue culture plates at 60% confluency and left to grow overnight before being treated with either 300 nM XL888, 3 μM AZD6244, 3 μM GDC-0941 (Selleck Chemical Co.) or the combination of 3 μM AZD6244 and 3 μM GDC-0941 for 72 or 144 hours. In some studies, RPMI7951 cells were treated with 300 nM XL888 in the presence or absence of 3 μM vemurafenib and harvested after 24, 48 and 72 hours Annexin V staining was performed as described in (Paraiso et al., Cancer Res. 71:2750-60 (2011)).

RNA Interference.

M229R and 1205LuR were plated at 1×10⁵ and left to grow overnight in RPMI complete media. The following day, complete media was replaced with Opti-MEM (Invitrogen; Carlsbad, Calif.) and Mcl-1 (25 nM Cell Signaling Technologies) or BIM (25 nM Cell Signaling Technology) siRNA's in complex with Lipofectamine 2000 (Invitrogen) were added. In addition, scrambled siRNA's were added as nontargeting controls. A final concentration of 5% FBS in complete RPMI was added the next day. In the BIM studies, cells were transfected for a total of 48 hours prior to a 48 hour treatment with 300 nM XL888. In the Mcl-1 studies, cells were transfected for a total of 96 hours prior to analysis.

Immunofluorescent Staining.

M229R and 1205LuR cells were seeded at 50% confluency onto glass coverslips in a 12 well plate and allowed to adhere overnight. The following day, cells were treated for 48 hours with 300 nM XL888, 3 μM AZD6244, 3 μM GDC-0941 or the combination of 3 μM AZD6244 and 3 μM GDC-0941 prior to fixation and permeabilized as previously described (Lyman et al., PLos ONE 6:e17692 (2011))). Fixed cells were separately stained by incubating overnight with rabbit anti-BIM or anti-FOXO3a antibodies followed by staining with secondary anti-rabbit AF488. Cells were then mounted onto slides with ProLong Gold Antifade containing DAPI (Invitrogen) and imaged with a Leica confocal microscope equipped with a 40× oil immersion lens.

Proteomics Sample Preparation.

Proteins were extracted as described for Western Blotting and processed as described in (Remily-Wood et al., Proteomics Clin. App. 5:383-96 (2011)).

Liquid Chromatography, Multiple Reaction Monitoring Mass (LC-MRM) Spectrometry (LC-MRM) Analysis.

LC-MRM was performed as described in (Remily-Wood et al., Proteomics Clin. App. 5:383-96 (2011)). Protein expression was determined using the ratio of peak area of the native peptide to corresponding internal standard; normalization of tissue results was performed using GAPDH to control for cellularity (Table 9). Data were then normalized to the pretreatment (cell lines) or vehicle controls (tissue) and plotted to show the changes in expression after drug treatment.

TABLE 9
Endogenous Peptides, Corresponding Internal
Standards, and Selected Transitions for
Quantification of Heat Shock Proteins.
Underlined amino acid residues are labeled
with ¹³C and ¹⁵N in the corresponding
synthetic standards.

| Protein | | |
|---|---|---|
| (UniProt | Endogenous | |
| Identifier) | Peptide | Transitions |
| HS90α | ALLFVPR | y₃-y₆ |
| | (SEQ ID NO: 3) | |
| HS90β | ALLFIPR | y₃-y₆ |
| | (SEQ ID NO: 4) | |
| H90β2 | HSQFLGYPITLYLEK | y₃-y₁₂ |
| | (SEQ ID NO: 5) | |
| CDC37 | LQAEAQQLR | y₄-y₇ |
| | (SEQ ID NO: 6) | |
| HSPB3 | ADLINNLR | y₃-y₇ |
| | (SEQ ID NO: 7) | |
| HSP71 | NQVALNPQNTVFDAK | y₃, y₄, |
| | (SEQ ID NO: 8) | y₉-y₁₂ |
| HSP72 | EIAEAYLGGK | y₃-y₈ |
| | (SEQ ID NO: 9) | |
| HSP7C | GTLDPVEK | y₃-y₇ |
| | (SEQ ID NO: 10) | |
| HSP74 | AFSDPFVEAEK | y₄, y₆-y₉ |
| | (SEQ ID NO: 11) | |
| GRP78 | VEIIANDQGNR | y₄-y₉ |
| | (SEQ ID NO: 12) | |

Human Specimen Procurement.

Patients scheduled to undergo surgical resection for metastatic melanoma were prospectively consented and accrued to an existing melanoma tissue procurement protocol approved by the Moffitt Cancer Center Scientific Review Committee and The University of South Florida Institutional Review Board. Following excision of the specimen in the operating room, fine needle tumor aspirates were taken using a 22-gauge needle for proteomic analysis of the resulting tumor homogenate.

MCL-1 Inducible Cell Line.

WM793TR MCL-1 cells were previously described (Boisvert-Adamo et al., Mol. Cancer. Res. 7:549-56 (2009)). Mcl-1 expression was induced by the addition of 100 ng/mL doxycycline for 72 hours prior to treatment with 300 nM XL888 for an additional 72 hours.

Quantitative Real-Time PCR.

Cells treated for 48 hours with 300 nM XL888, 3 μM AZD6244, 3 μM GDC-0941 or the combination of 3 μM AZD6244 and 3 μM GDC-0941 prior to RNA isolation. Total RNA was isolated using Qiagen's RNeasy mini kit. The following TaqMan® Gene Expression Assays primer/probes were used: Hs00197982_m1 (BIM), Hs01050896_m1 (MCL-1), Hs00372937_m1 (BMF), Hs00818121_m1 (FOXO3a), P/N 4319413E (18S) and Hs99999905_m1 (GAPDH). The 18S+GAPDH data were used for normalizing BIM. Q-RT-PCR reactions were performed as previously described (Paraiso et al., Cancer Res. 71:2750-60 (2011)).

Colony Formation.

Cells (1×10⁴ per ml) were grown overnight before being treated with vehicle (DMSO) or XL888 (300 nM) for 4 weeks as described in (Paraiso et al., Br. J. Cancer 102:1724-30 (2010)), and relative colony density was determined by solubilizing the crystal violet dye in 10% acetic acid followed by measurement of absorbance at 450 nm.

Xenograft Experiments.

BALB SCID mice (The Jackson Laboratory, Bar Harbor, Me.) were subcutaneously injected with 2.5×10⁶ cells per mouse (resuspended in 111 μL L-15 media, 10 mM HEPES, 37.5 μL Matrigel). Tumors were grown to approximately 100 mm³ prior to dosing. Mice were treated with either 100 mg XL888/kg (n=5) or an equivalent volume of vehicle (10 mM HCl), 3 times per week by oral gavage. Mouse weights and tumor volumes (L×W2/2) were measured 3 times per week. Upon completion of the experiment, vehicle and drug treated tumor biopsies were processed for LC-MRM analysis (as above).

3D Spheroid Assays.

Melanoma spheroids were prepared using the liquid overlay method (Smalley et al. Mol Cancer Ther. 5: 1136-1144 (2006)). Spheroids were treated for 144 hours with either vehicle or 1 μM XL888 before being washed (3 times in media) and treated with calcein-AM and ethidium bromide (Molecular Probes, Eugene, Oreg.) for 1 hour at 37° C., according to the manufacturer's instructions. Pictures of the invading spheroids were taken using a Nikon-300 inverted fluorescence microscope.

Measurement of Proteasome Activity.

M229R and 1205LuR cells were harvested and plated at 7,500 cells per well in a white-walled 96 well plate. Cells were allowed to grow overnight prior to treatment with either 0.3 μM XL888, 3 μM AZD6244, 3 μM GDC-0941 or the combination of 3 μM AZD6244 and 3 μM GDC-0941 (48 hours) or MG-132 (0.3, 1 or 3 μM for 24 hours). Proteasome activity was assessed using the Proteasome-Glo Chymotrypsin-Like Cell Based Assay (Promega; Madison, Wis.) and was measured in relative luminescence units (RLU).

Statistical Analysis of GI50 Values.

Triplicate experiments were performed for each cell line under each drug treatment. To estimate the IC₅₀ values for each cell line for each treatment, a 4-parameter Hill equation was used to model the nonlinear sigmoid relationship between the drug concentration and % cell survival. Briefly,

$$y = \frac{( E - B ) \cdot {( \frac{x}{{GI}_{50}} )}^{m}}{1 + {( \frac{X}{{GI}_{50}} )}^{m}} + B + ɛ ,$$

where x is the drug concentration, y is the % survival, ε is the error term. The four parameters in the model to estimate are: 1) the control-level effect (E); 2) the background-level effect (B); 3) the median effect concentration (GI₅₀); and 4) the strength of the inhibition (m). The constraint of non-negative minimum background effect (B) is imposed so the model is biologically meaningful. Levenberg-Marquardt algorithm for nonlinear least squares was used to fit the model and estimate all four parameters for each cell line under each treatment. Estimated concentration at 50% cell survival, i.e., IC₅₀, is estimated using the plugged-in parameters after model fitting. Note that when the control and background effects are 100% and 0%, respectively, the estimated GI₅₀ and IC₅₀ values would be identical. To compare the estimated IC₅₀ values between the resistance and naïve paired of cell lines, after log transformation, a paired t-test was performed for each drug.

Statistical Analysis.

Data show the mean of at least three independent experiments±the S.E. mean, unless stated otherwise. Statistically significant results were considered where P≦0.05. Additional statistical analyses are described in the supplemental material.

Results

Inhibition of HSP90 Overcomes Resistance to Vemurafenib Resistance Mediated Through Diverse Mechanisms.

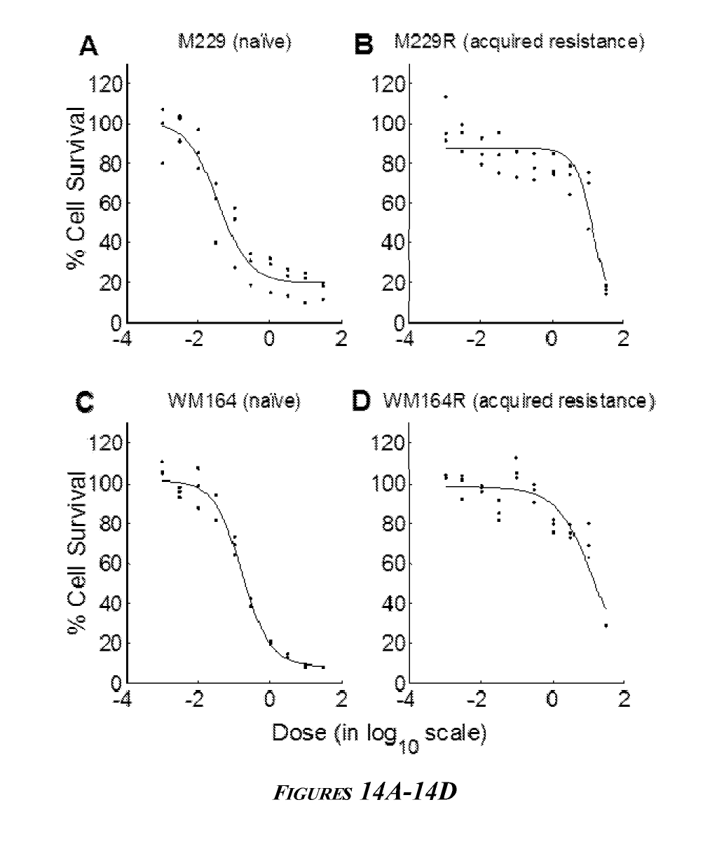

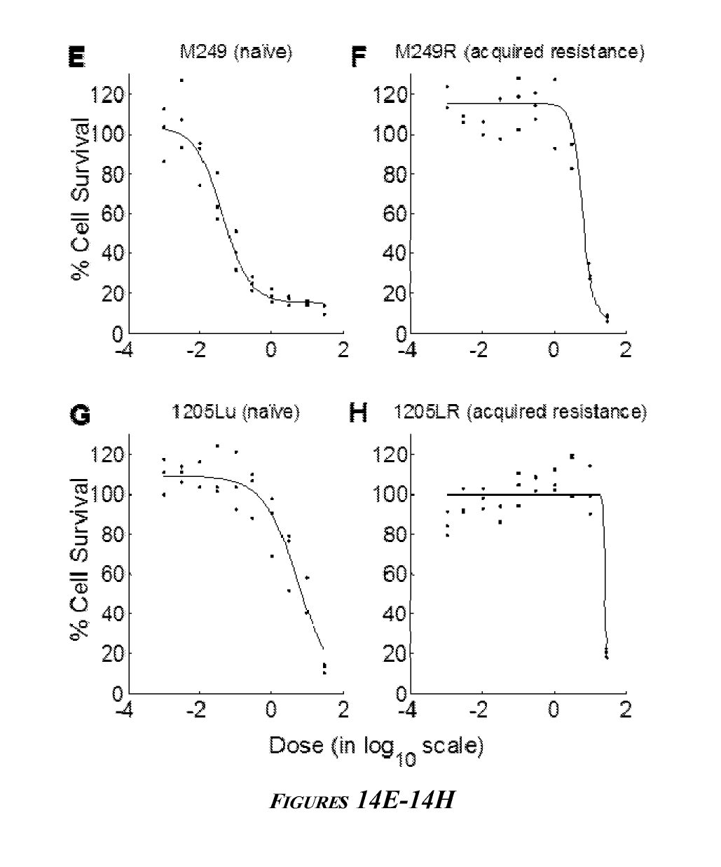

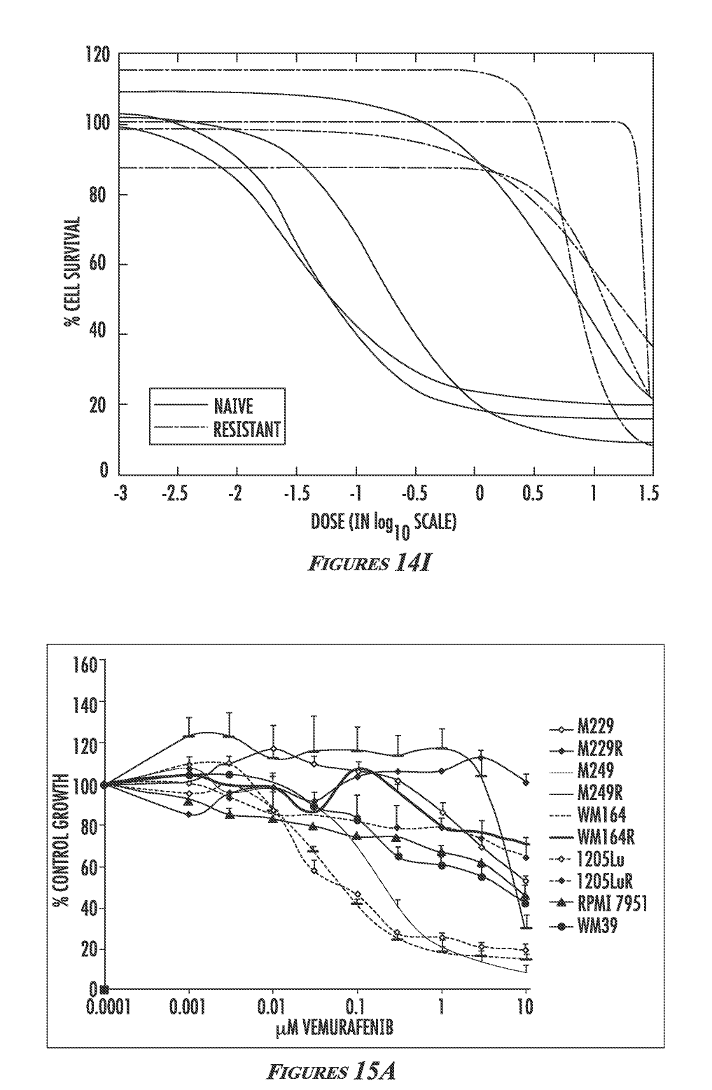

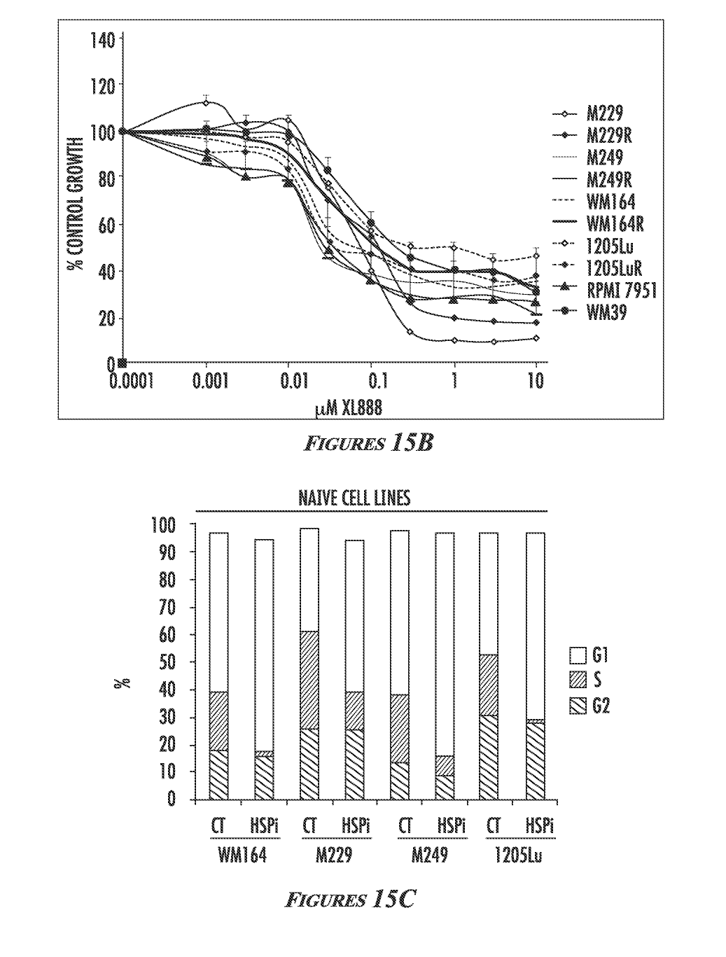

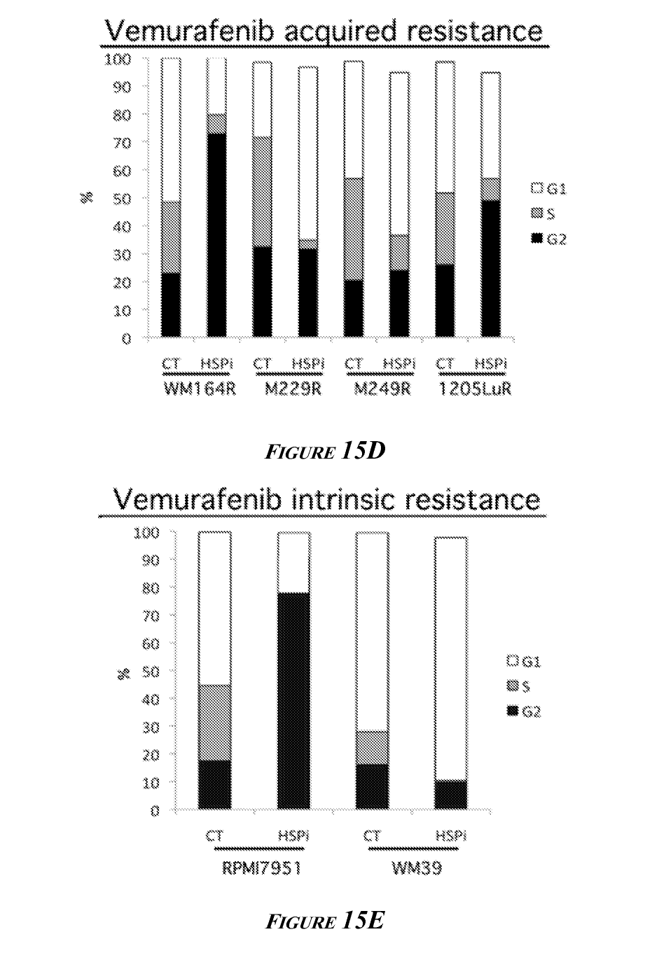

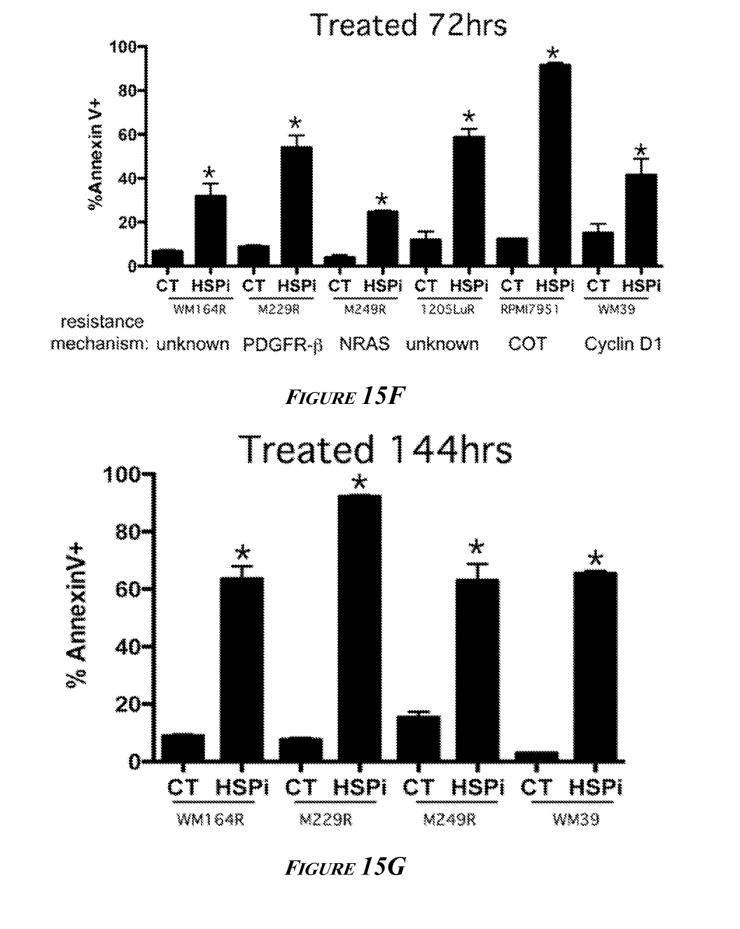

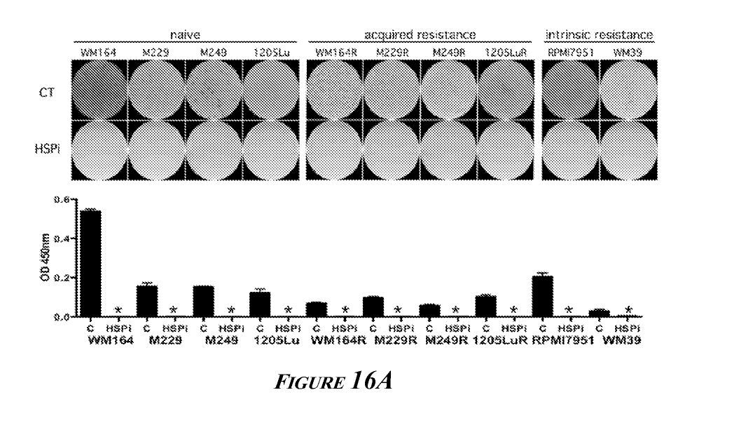

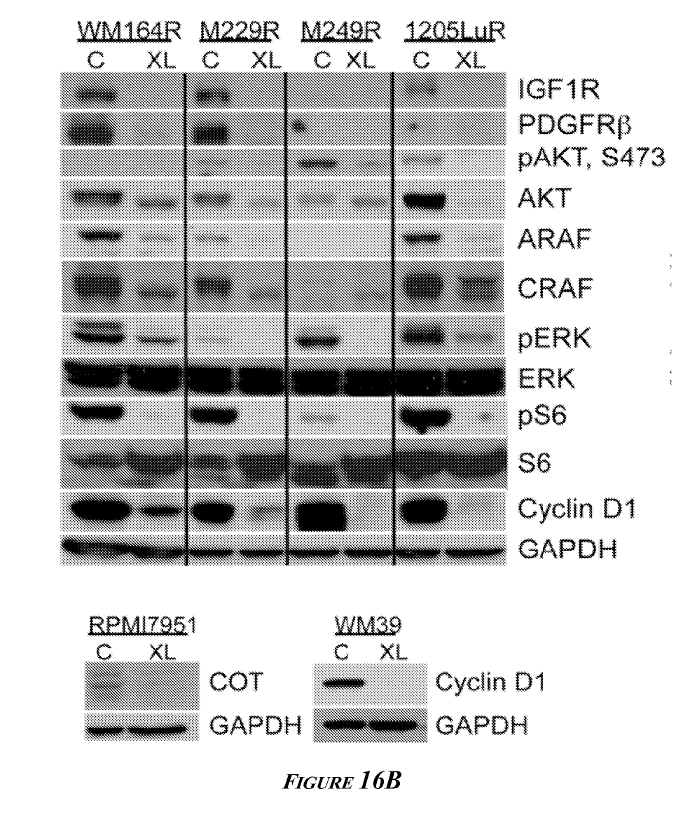

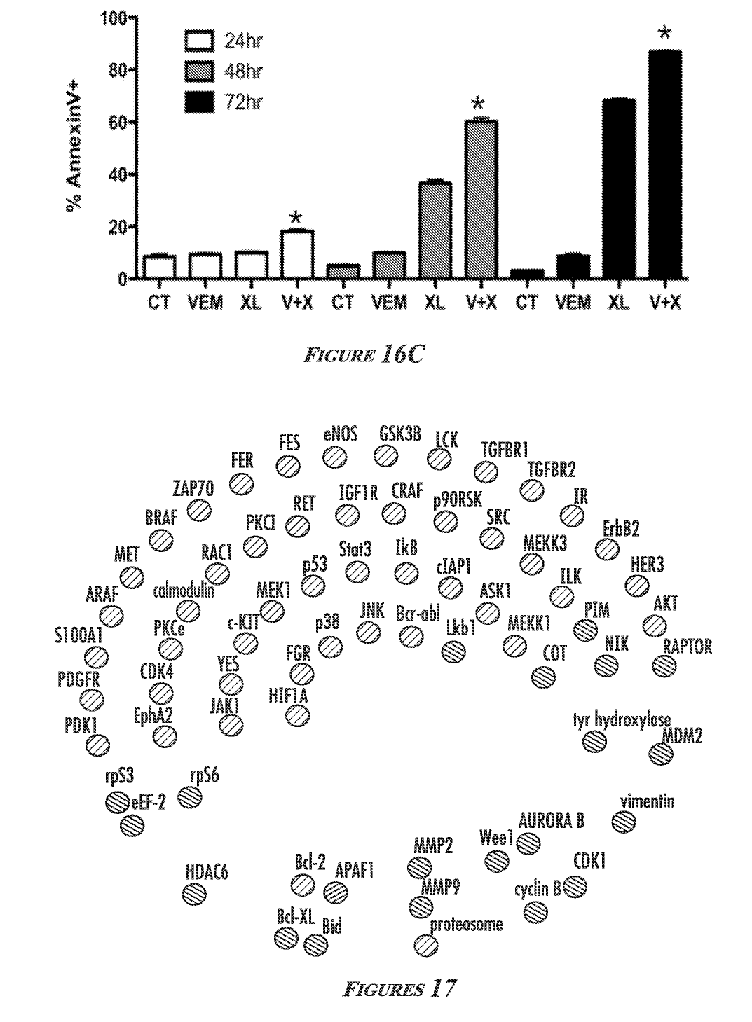

A panel of BRAF V600E mutant melanoma cell lines with different mechanisms of intrinsic resistance and acquired vemurafenib resistance was assembled (Table 10). Treatment of matched BRAF inhibitor naïve and resistant melanoma cell lines with vemurafenib showed a statistically significant difference in the extent of growth inhibition (P=0.02; t=−0.4.38; FIG. 14) when resistance was mediated through increased PDGFRβ expression (M229R), and an acquired NRAS mutation (M249R), as well as two lines with uncharacterized mechanisms of resistance (WM164R and 1205LuR) (FIGS. 15A, 15B). Cell lines with amplification of cyclin D1 (WM39) and overexpression of COT (RPMI 7951) showed signs of intrinsic resistance to vemurafenib (IC₅₀>304). By contrast, treatment of with the HSP90 inhibitor XL888 led to dose dependent decreases in the growth of all the cell lines with no significant difference in IC₅₀ values observed between the naïve and resistance pairs of cell lines (t=0.25, p=0.82) (FIGS. 15C, 15E). The growth inhibitory effects of XL888 were associated with induction of either a G1-phase cell cycle arrest (WM164, M229, M229R, M249, M249R, 1205Lu, WM39) or a G2/M phase cell cycle arrest (WM164R, 1205LuR, RPMI 7951) (FIG. 15C, 15E). Treatment of all of the vemurafenib resistant melanoma cell lines with XL888 (300 nM) induced high levels (>66%) of apoptosis in every cell line tested (FIG. 15F, 15G). The cytotoxic effects of XL888 were durable with no signs of colony formation observed in any of the cell lines (up to 4 weeks: FIG. 16A).

TABLE 10
List of cell lines with mechanisms of resistance included.

| Cell line | Resistance mechanism |
|---|---|
| WM164R | Unknown |
| 1205LuR | Unknown |
| M229R | PDGFR-β overexpression |
| M249R | NRAS mutation |
| WM39 | Cyclin D1 amplification |
| RPMI7951 | COT overexpression |

Inhibition of HSP90 Degrades all of the Proteins Identified as being Critical for Vemurafenib Resistance.

Whether XL888 treatment induced the degradation of all the signaling mediators implicated in acquired and intrinsic resistance was investigated (FIG. 17 summarizes melanoma-relevant HSP90 clients). XL888 treatment (300 nM, 48 hours) led to the degradation of IGF1R, PDGFRβ, ARAF, CRAF and cyclin D1 and the inhibition of AKT, ERK and S6 signaling in all of the cell lines with acquired BRAF inhibitor resistance (FIG. 16B). In the intrinsically vemurafenib-resistant melanoma cell lines RPMI7951 and WM39, XL888 treatment was found to degrade both COT and cyclin D1, respectively (FIG. 16B). In line with the observation that COT mediates resistance to vemurafenib (Johannessen et al., Nature 468:968-72 (2010)), the combination of XL888 with vemurafenib significantly enhanced levels of apoptosis observed in RPMI7951 cells, compared to XL888 alone (FIG. 16C). Because the microenvironment modulates the response of melanoma cells to targeted therapies (Smalley et al., Mol. Cancer Ther. 5:1136-44 (2006)), the panel of vemurafenib-resistant cell lines were grown as collagen implanted 3D spheroids, and it was noted that XL888 was effective at inducing cell death (FIG. 18).

Development of a Quantitative Pharmacodynamic Assay of HSP90 Inhibition.

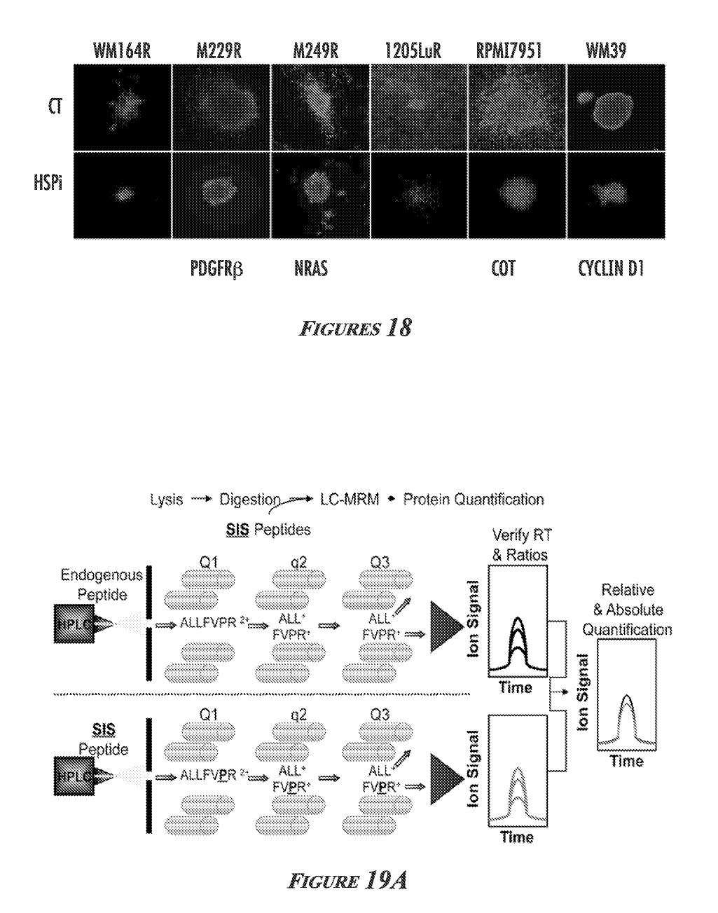

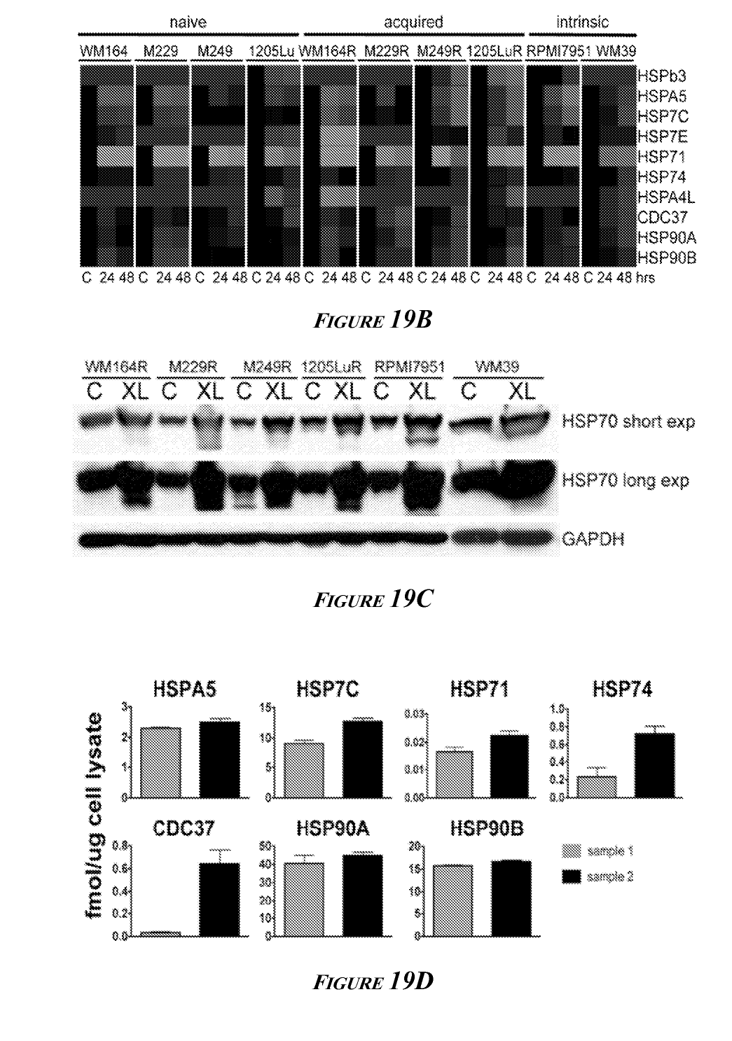

The clinical development of HSP90 inhibitors has been hampered by the lack of a good pharmacodynamic assay for quantifying target inhibition within the tumor (Trepel et al., Cancer 10:537-49 (2010)). As inhibition of HSP90 typically leads to the increased expression of other HSP family members which can be used as a surrogate for HSP90 inhibition, a highly sensitive quantitative LC-MRM assay for the quantification of 11 HSP family members was developed (FIG. 19A). Treatment of cell lines that were naïve, intrinsically resistant and with acquired vemurafenib resistance with XL888 (300 nM) led to robust time-dependent increases in the expression of HSP70 isoform 1 (HSP71) (FIG. 19B). Western blot experiments confirmed the XL888-dependent increases in HSP70 expression in every cell line evaluated (FIG. 19C). The potential clinical relevance of the LC-MRM assay was demonstrated by the successful quantification of HSP70 and other chaperone proteins from fine needle aspirates (˜2000 cells) taken from two melanoma specimens (FIG. 19D).

XL888 Treatment Causes the Regression of Vemurafenib-Resistant Xenografts In Vivo Associated with Increased Intratumoral HSP70 Expression.

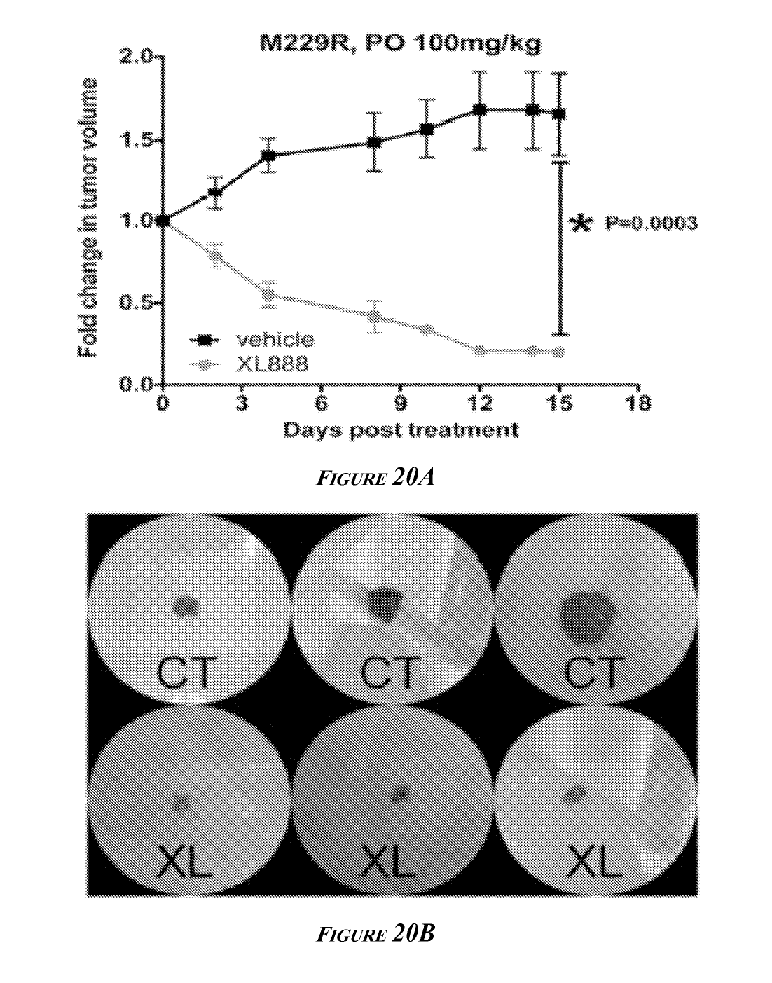

The relevance of HSP90 inhibition as a strategy to overcome BRAF inhibitor resistance in vivo was demonstrated by the ability of XL888 (100 mg/kg, PO, 3× week) to significantly induce the regression of established M229R xenografts in SCID mice (FIGS. 20A and 20B). LC-MRM mediated analysis of xenograft samples following 15-days of XL888 treatment showed a robust (8.6-fold) increase in intratumoral HSP70 expression compared to controls (FIG. 20C).

HSP90 Inhibition Restores Nuclear Localization of FOXO3a, Upregulates BIM Expression and Inhibits Mcl-1 Expression in Vemurafenib-Resistant Cell Lines.

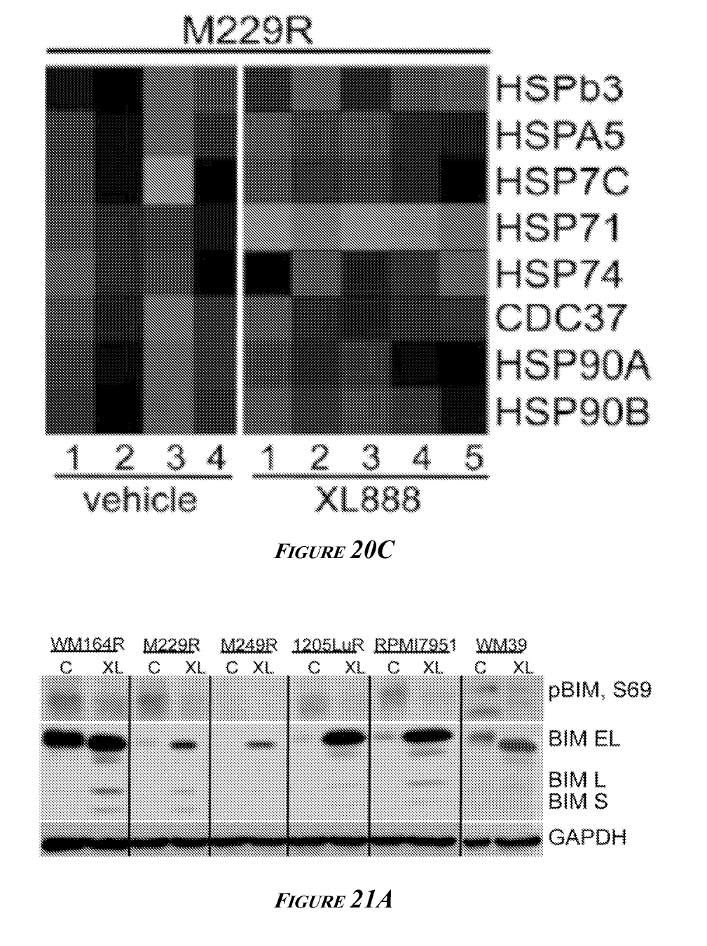

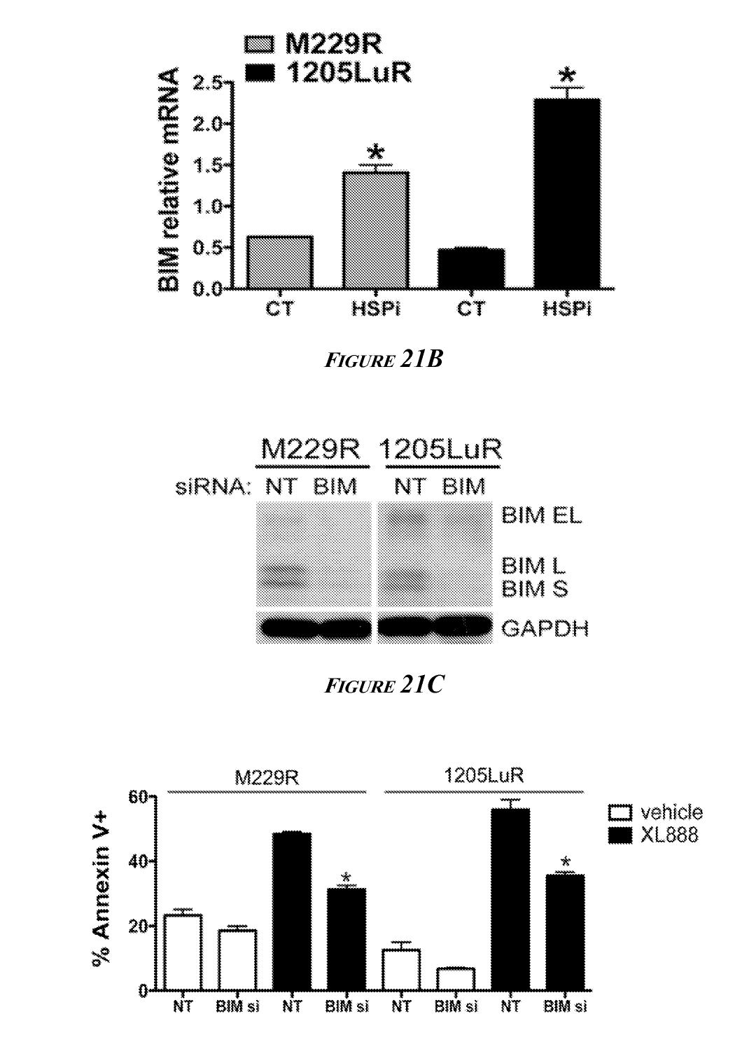

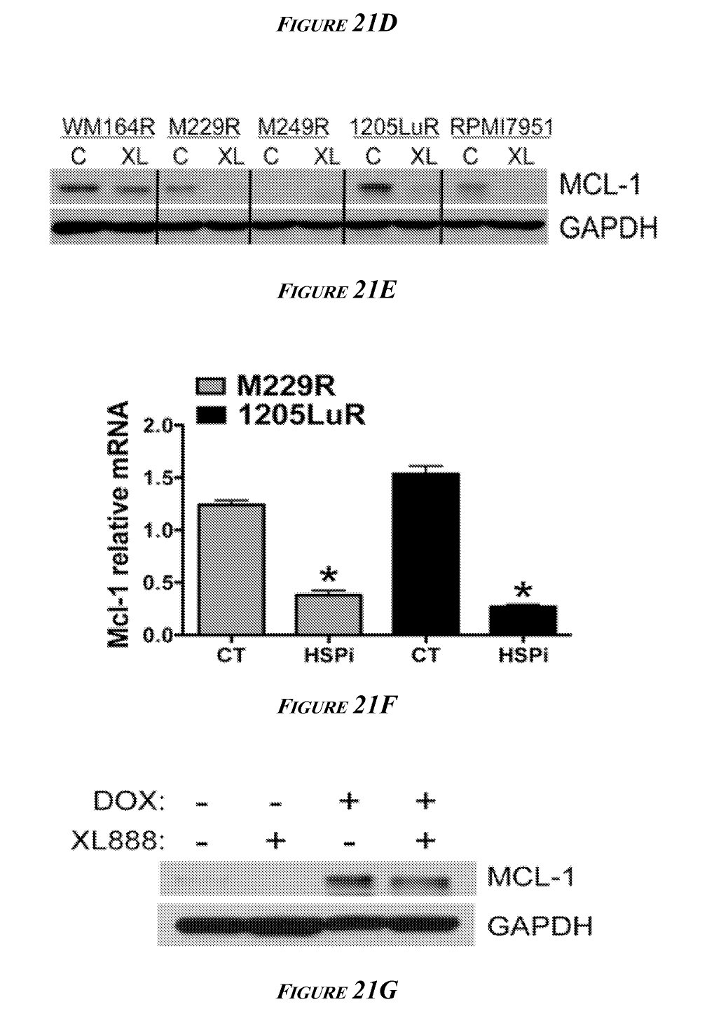

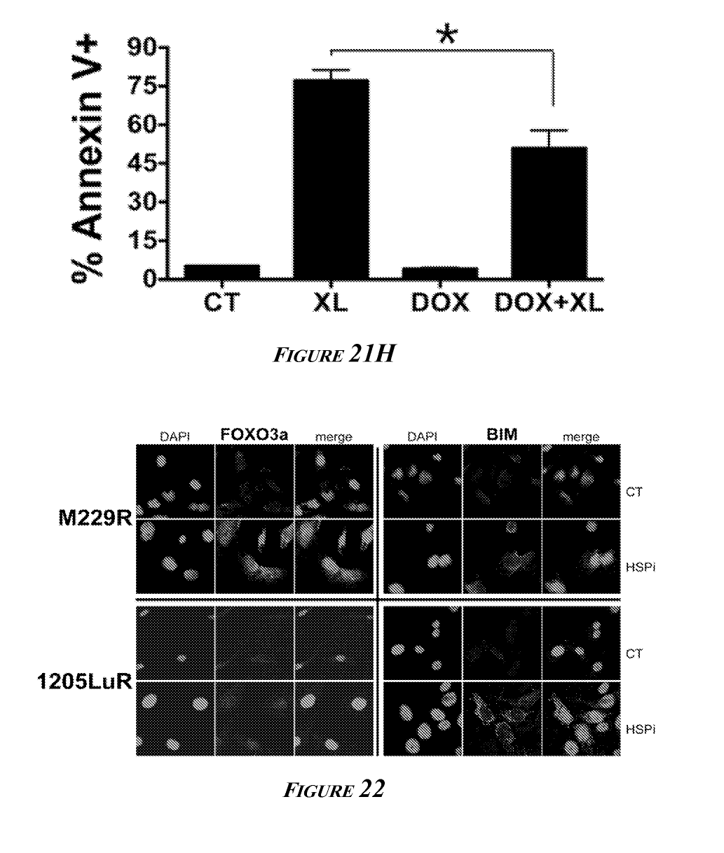

To determine the mechanism of XL888-induced apoptosis in the vemurafenib-resistant melanoma cell lines, BIM was first investigated. Whereas vemurafenib treatment increased expression of BIM in melanoma cell lines that were drug naïve (Paraiso et al., Cancer Res. 71:2750-60 (2011)), the resistant cell lines suppressed their expression of BIM even in the continuous presence of vemurafenib (FIG. 21A, 21B). XL888 treatment reversed this and increased BIM expression across the entire cell line panel, irrespective of resistance mechanism (FIG. 21A, 21B). These effects were mediated in part through increased BIM protein stability as noted by decreased BIM phosphorylation at Ser69 in all of the cell lines tested apart from M249R (FIG. 21A). Whether HSP90 inhibition also affected BIM expression at the mRNA level was next investigated. In vemurafenib naïve cells, inhibition of BRAF leads to the nuclear accumulation of the transcription factor FOXO3a and increased BIM expression (Paraiso et al., Cancer Res. 71:2750-60 (2011)). In contrast, cell lines with acquired resistance to vemurafenib excluded FOXO3a from the nucleus and suppressed BIM protein and mRNA expression even in the continuous presence of vemurafenib (FIGS. 21A and 22). XL888 treatment reversed these effects and led to the nuclear accumulation of FOXO3a and an increase in BIM mRNA and protein expression (FIGS. 21A, 21B, and 22). The importance of BIM expression in the XL888-mediated cell death response was demonstrated by the significant inhibition of apoptosis observed when BIM expression was knocked down by siRNA (FIG. 21C, 21D).

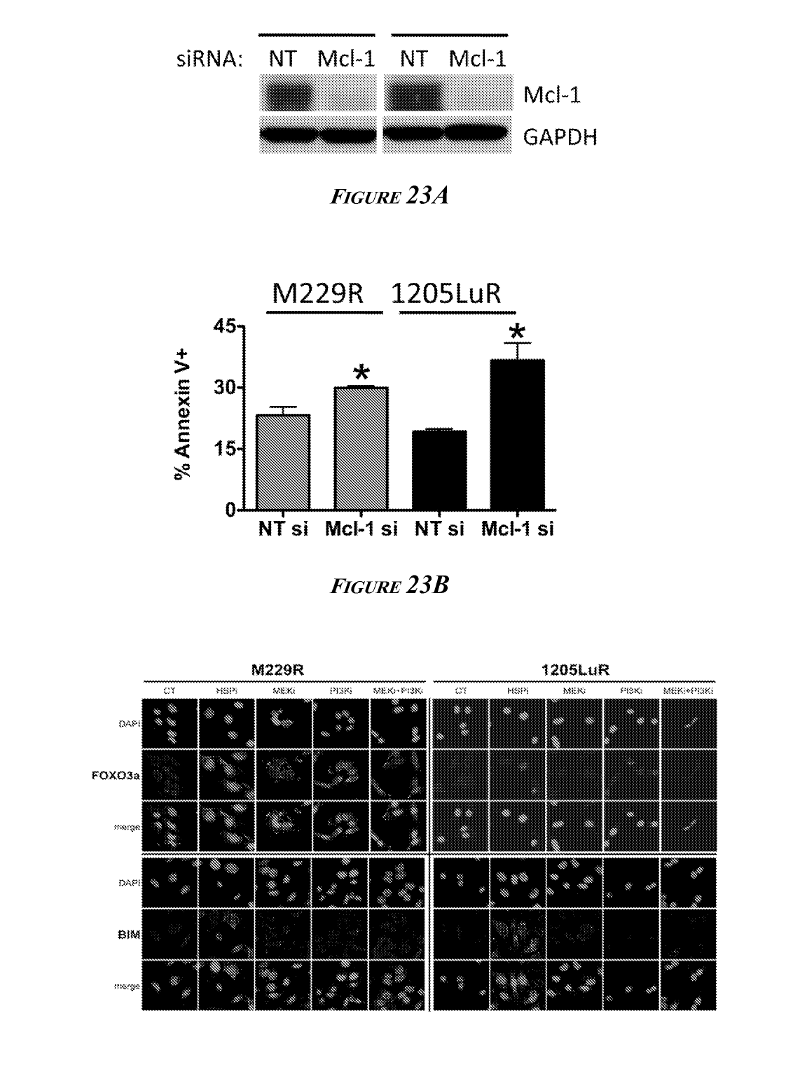

Mcl-1 is pro-survival BH3 family protein member that antagonizes the activity of BIM (Biosvert-Adamo et al., Mol. Cancer Res. 7:549-56 (2009)). Treatment of melanoma cell lines in which vemurafenib resistance was mediated through PDGFRβ, COT overexpression and two melanoma cell lines with unknown resistance mechanisms with XL888 (300 nM, 48 hours) led to a marked decrease in the expression of Mcl-1 (FIG. 21E). Quantitative RT-PCR experiments showed that XL888 treatment also blocked Mcl-1 expression at the mRNA level (FIG. 21F). The importance of Mcl-1 expression for the survival of vemurafenib-resistant melanoma cell lines was confirmed by the significant induction of apoptosis observed following siRNA knockdown of Mcl-1 expression (FIG. 23). Further evidence for the role of Mcl-1 expression in the drug resistance phenotype came from overexpression studies in which induction of Mcl-1 expression following doxycycline treatment led to a significant reduction in the magnitude of XL888-induced apoptotic response (FIG. 21G, 21H).

HSP90 Inhibition is More Effective at Inducing BIM Expression and Apoptosis than Combined MEK+PI3K Inhibition.

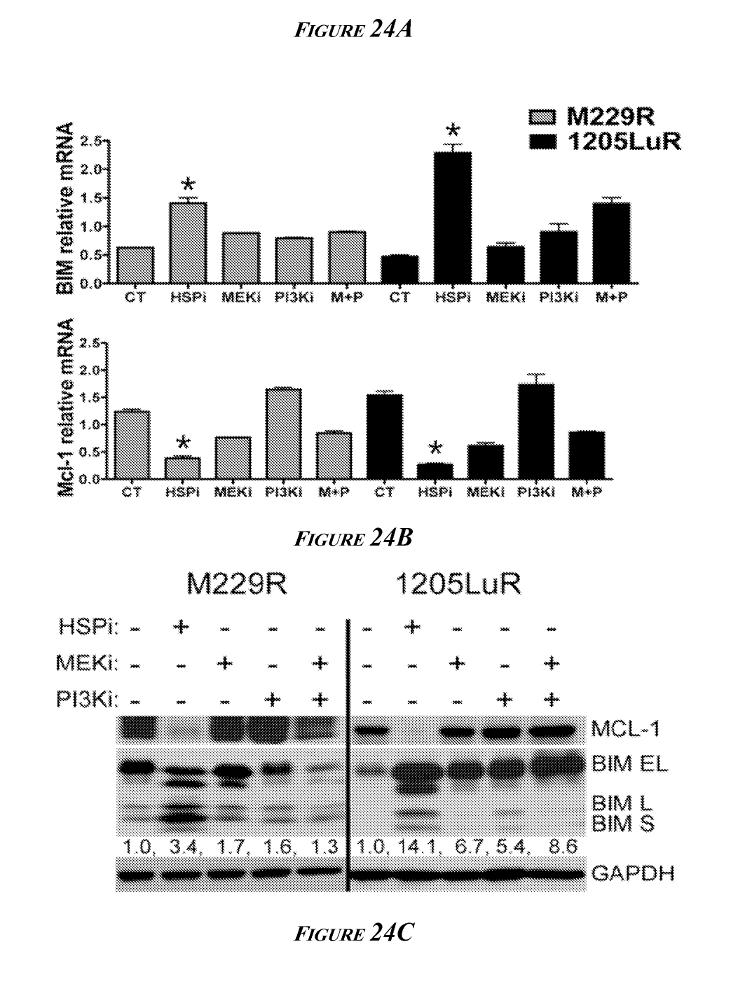

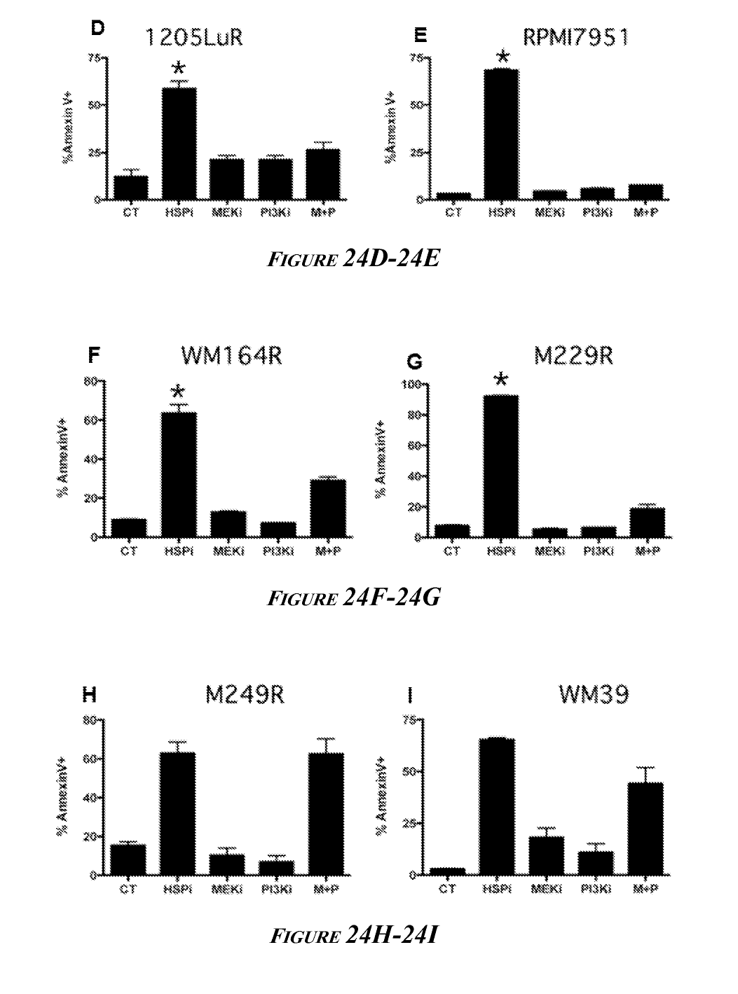

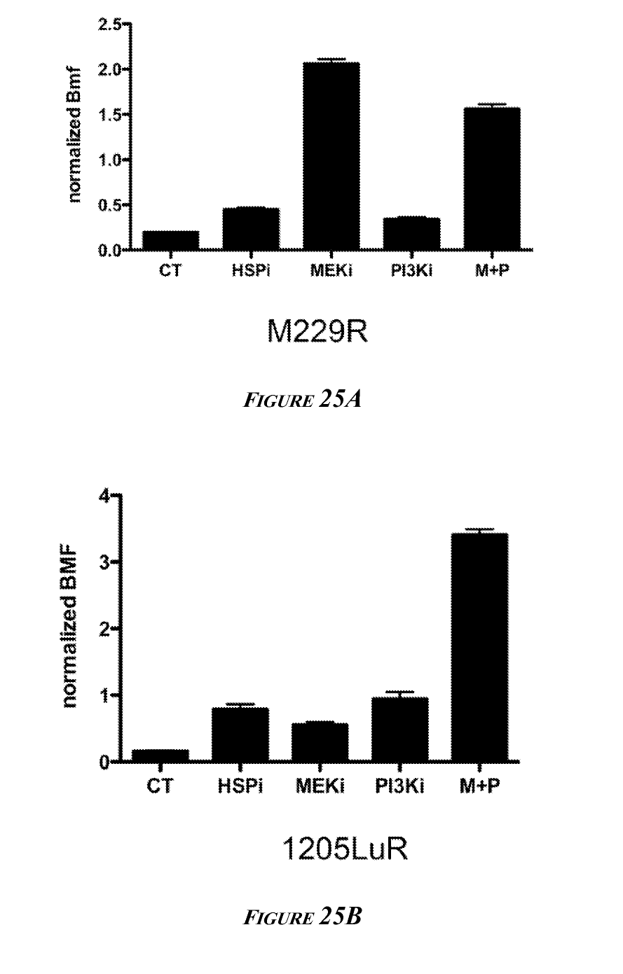

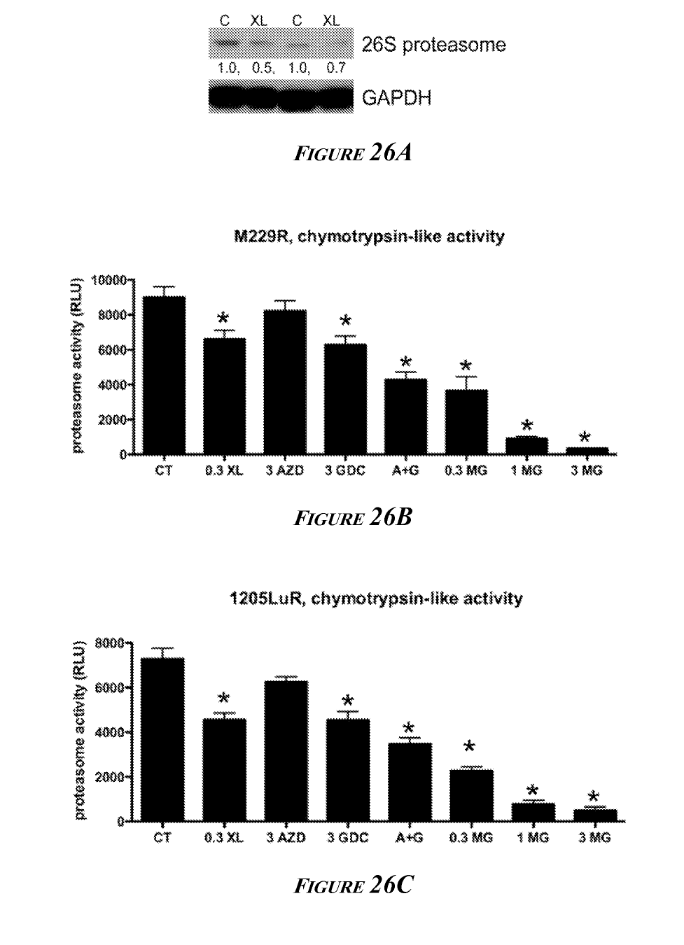

One potential strategy for overcoming vemurafenib resistance is the simultaneous targeting of MEK/ERK and PI3K/AKT signaling. Whether HSP90 inhibition was more effective than the MEK+PI3K inhibitor combination at restoring apoptosis in vemurafenib-resistant melanoma cells was investigated. Although both XL888 and the PI3K inhibitor GDC-0941 were highly efficient at increasing nuclear accumulation of FOXO3a (FIG. 24A), XL888 treatment led to a greater induction of BIM expression at both the protein and mRNA levels and significantly restored the apoptotic response (FIGS. 24B and 24C). Similarly, XL888 treatment was also more effective than the MEK or PI3K inhibitor, alone or in combination, at downregulating the expression of Mcl-1 at both the mRNA and protein levels (FIGS. 24B and 24C). Although there is evidence that the BH3 protein family member BMF plays a role in the apoptotic response to BRAF inhibition (Shao et al., 70:6670-81 (2010)), XL888 treatment only weakly induced BMF mRNA expression (FIG. 25). In contrast, treatment of two vemurafenib-resistant cell lines with either the MEK inhibitor (M229R) or the MEK+PI3K inhibitor (1205LuR) led to a robust induction of BMF expression but induced less apoptosis than following XL888 treatment (FIGS. 24D-24I, and 25). As the phosphorylation of BIM by MEK/ERK leads to its proteasomal degradation and the 26S proteasome is an HSP90 client protein, the contribution of proteasome inhibition to the cytotoxic effects of XL888 was determined next. Although XL888 treatment was observed to partly degrade the 26S proteasome, HSP90 inhibition had a considerably weaker effect upon proteasomal activity than either the MEK+PI3K inhibitor combination or the proteasome inhibitor (MG-132) (FIG. 26). In agreement with the marked effects of HSP90 inhibition on BIM and Mcl-1 expression compared to the MEK, PI3K and MEK+PI3K inhibitor combination, XL888 was observed to induce significantly higher levels of apoptosis than each of the other drug combinations in cell lines where resistance was mediated through amplification of COT, PDGFRβ overexpression and in two other models where the resistance mechanism is as yet unknown (FIG. 24D-24I). The level of apoptosis induced by the MEK+PI3K inhibitor combination was equivalent to that of the HSP90 inhibitor when resistance was mediated through NRAS mutation or cyclin D1 amplification (FIG. 24D-24I).

### Example 4

### High-Throughput Quantification of Heat Shock Proteins and Immunoglobulins

High-throughput methods have been devised for measuring protein expression using direct infusion-multiple reaction monitoring mass spectrometry (DI-MRM). These methods were appropriate for rapid measurements of biomarkers that enable evaluation of tumor burden or assessment of response and resistance to drug treatment. While protein quantification with liquid chromatography-multiple reaction monitoring mass spectrometry (LC-MRM) usually takes 30-60 minutes, DI-MRM measurements can be completed on the seconds to minutes time scale (typically <2 minutes). These methods enable the assessment of large patient cohorts without the purchase of multiple mass spectrometers. An Advion Nanomate 100 automated nanospray infusion ion source interfaced with a Thermo Vantage triple quadrupole was used for these measurements, but similar equipment is suitable.

Two cases have been explored. In the first, the expression of heat shock proteins (HSPs) in cell lysates was quantified to enable the assessment of response and resistance to heat shock protein inhibition. As an example, heat shock proteins are a drug target of interest for cancer treatment. These chaperones assist in protein folding and stability, and they are even more important for maintenance of mutant proteins that support tumorigenesis and cancer progression. While HSP90 and HSP70 isoform 1 (HSP71) have been the focus for biology and treatment interventions, a biomarker panel of heat shock proteins has been developed that can be assessed to examine HSP inhibitor response. Of note, HSP 71 increases after HSP90 inhibition. For each protein, several peptides have been detected by LC-MRM screening and DI-MRM assays have been developed for selected peptides as described throughout. In addition, the housekeeping protein, GAPDH, is used to assess tumor cellularity when applied to patient samples.

TABLE 11
LC-MRM and DI-MRM Assays Developed for Measuring the Expression of Heat
Shock Proteins. For each protein, the Uniprot accession, peptide(s),
label, and transitions for LC-MRM analysis are listed. Transitions for
DI-MRM were selected using a cutoff value of 40% for interference con-
tribution from other ion signals observed in that transition during LC-
MRM. GAPDH was used as a control for evaluation of protein loading.

| Protein | | | LC-MRM | DI-MRM |
|---|---|---|---|---|
| (Uniprot) | Peptide | Label | Transitions | Transitions |
| HS90A | ALLFVPR | P₆: ¹³C₅, ¹⁵N | y₃-y₆ | y₃-y₅ |
| | (SEQ ID NO: 3) | | | |
| | EQVANSAFVER | V₉: ¹³C₅, ¹⁵N | y₄-y₉ | y₅, y₆, |
| | (SEQ ID NO: 18) | | | y₈, y₉ |
| HS90B | ALLFIPR | P₆: ¹³C₅, ¹⁵N | y₃-y₆ | y₃-y₅ |
| | (SEQ ID NO: 4) | | | |
| | DQVANSAFVER | V₉: ¹³C₅, ¹⁵N | y₄-y₉ | y₅-y₉ |
| | (SEQ ID NO: 15) | | | |
| CDC37 | LQAEAQQLR | L₈: ¹³C₆, ¹⁵N | y₄-y₇ | y₇ |
| | (SEQ ID NO: 6) | | | |
| | EGEEAGPGDPLLEAVPK | P₁₆: ¹³C₅, ¹⁵N | y₄, y₅, y₈, y₉, | y₈, |
| | (SEQ ID NO: 19) | | y₁₁-y₁₃ | y₁₁-y₁₃ |
| HSP71 | NQVALNPQNTVFDAK | V₁₁: ¹³C₅, ¹⁵N | y₃, y₄, y₉-y₁₂ | y₃, y₄, |
| | (SEQ ID NO: 8) | | | y₉-y₁₂ |
| HSP7C | GTLDPVEK | V₆: ¹³C₅, ¹⁵N | y₃-y₇ | y₄, y₆ |
| | (SEQ ID NO: 10) | | | |
| HSP74 | AFSDPFVEAEK | V₇: ¹³C₅, ¹⁵N | y₄, y₆-y₉ | y₇, y₉ |
| | (SEQ ID NO: 11) | | | |
| HSPA5 | VEIIANDQGNR | V₁: ¹³C₅, ¹⁵N | y₄-y₉ | y₇, y₉ |
| | (SEQ ID NO: 12) | | | |
| HSP7E | FTVLFPSGTPLPAR | P₁₂: ¹³C₅, ¹⁵N | y₃, y₅, y₇-y₁₁ | y₉-y₁₁ |
| | (SEQ ID NO: 20) | | | |
| GAPDH | VGVNGFGR | G₇ → A | y₃-y₇ | y₃-y₇ |
| | (SEQ ID NO: 21) | | | |

### Additional Peptides

| | |
|---|---|
| GRP78: | |
| VTHAVVTVPAYFNDAQR, | (SEQ ID NO: 26) |
| ELEEIVQPIISK, | (SEQ ID NO: 27) |
| TFAPEEISAMVLTK, | (SEQ ID NO: 28) |
| TWNDPSVQQDIK, | (SEQ ID NO: 29) |
| ITPSYVAFTPEGER, | (SEQ ID NO: 30) |
| SQIFSTASDNQPTVTIK, | (SEQ ID NO: 31) |
| NQLTSNPENTVFDAK | (SEQ ID NO: 32) |
| H90β2_HUMAN: | |
| ADLINNLGTIAK, | (SEQ ID NO: 33) |
| HSQFLGYPITLYLEK, | (SEQ ID NO: 34) |
| YESLTDPSK, | (SEQ ID NO: 35) |
| SIYYITGESK | (SEQ ID NO: 36) |
| HS74L_HUMAN: | |
| EDISSIEIVGGATR, | (SEQ ID NO: 37) |
| SFDDPIVQTER | (SEQ ID NO: 38) |
| HS90α_HUMAN: | |
| NPDDITNEEYGEFYK, | (SEQ ID NO: 13) |
| LGIHEDSQNR, | (SEQ ID NO: 16) |
| DQVANSAFVER, | (SEQ ID NO: 15) |
| FYEQFSK | (SEQ ID NO: 14) |
| HS90β_HUMAN: | |
| FYEAFSK, | (SEQ ID NO: 39) |
| EQVANSAFVER, | (SEQ ID NO: 18) |
| NPDDITQEEYGEFYK, | (SEQ ID NO: 13) |
| HLEINPDHPIVETLR | (SEQ ID NO: 40) |
| HSP71_HUMAN: | |
| LLQDFFNGR, | (SEQ ID NO: 41) |
| FELSGIPPAPR, | (SEQ ID NO: 42) |
| IINEPTAAAIAYGLDR, | (SEQ ID NO: 43) |
| TTPSYVAFTDTER, | (SEQ ID NO: 44) |
| LIGDAAK | (SEQ ID NO: 45) |
| HSP74_HUMAN: | |
| EDIYAVEIVGGATR, | (SEQ ID NO: 46) |
| EFSITDVVPYPISLR, | (SEQ ID NO: 47) |
| ELSTTLNADEAVTR, | (SEQ ID NO: 48) |
| SNLAYDIVQLPTGLTGIK | (SEQ ID NO: 49) |
| HSP7C_HUMAN: | |
| EIAEAYLGK, | (SEQ ID NO: 50) |
| TTPSYVAFTDTER, | (SEQ ID NO: 51) |
| DAGTIAGLNVLR, | (SEQ ID NO: 52) |
| FELTGIPPAPR, | (SEQ ID NO: 53) |
| SQIHDIVLVGGSTR | (SEQ ID NO: 54) |
| HSP7E_HUMAN: | |
| AAGFNVLR, | (SEQ ID NO: 55) |
| YEIDTGEETK, | (SEQ ID NO: 56) |
| FAQVVLQDLDK, | (SEQ ID NO: 57) |
| FVNPEDVAR | (SEQ ID NO: 58) |
| HSPB1_HUMAN: | |
| DWYPHSR, | (SEQ ID NO: 59) |
| QLSSGVSEIR, | (SEQ ID NO: 60) |
| DGVVEITGK, | (SEQ ID NO: 61) |
| LFDQAFGLPR, | (SEQ ID NO: 62) |
| VSLDVNHFAPDELTVK, | (SEQ ID NO: 63) |
| LATQSNEITIPVTFESR, | (SEQ ID NO: 64) |
| GPSWDPFR, | (SEQ ID NO: 65) |
| VPFSLLR | (SEQ ID NO: 66) |
| HSPB2_HUMAN: | |
| TVDNLLEVSAR | (SEQ ID NO: 67) |
| CDC37_HUMAN: | |
| LGPGGLDPVEVYESLPEELQK, | (SEQ ID NO: 68) |
| EGEEAGPGDPLLEAVPK | (SEQ ID NO: 69) |

In the second case, the ability to quantify immunoglobulin expression for the ongoing assessment of multiple myeloma patients and other patients with plasma cell dyscrasias or monoclonal gammopathy of undetermined significance (MGUS) was determined. In this case, the amount of the antibody is quantified as part of the diagnostic and prognostic evaluation of the patient. In the US, there are approximately 50,000 patients living with multiple myeloma; based on their response to therapy, they live on average 2.5 to 5 years and require monitoring on a monthly basis (if not more frequently). MGUS is much more prevalent; as the population ages, the incidence increases from 3% at age 50 to 7% at age 85. MGUS patients progress to multiple myeloma at a rate of 1% per year. DI-MRM analysis of these patients provides high-throughput processing of large volumes of patient samples. A central facility could serve the entire country with a minimum number of mass spectrometers due to the high speed of sample analysis. As previously described for LC-MRM, panel of peptide biomarkers representative of the immunoglobulins and serum albumin has been developed for DI-MRM.

TABLE 12

| | Average | | | |
|---|---|---|---|---|
| | Expres- | | | |
| | sion & | | | |
| | Reference | | | |
| Pro- | Range | | | Transi- |
| tein | (mg/ml) | Peptide | IS | tions |
| IgG1 | 5.91⁷ | GPSVFPLAPSSK | 10.2 | y₈-y₁₀ |
| | 3.19-10.2 | (SEQ ID NO: 70) | | |
| IgG2 | 3.04⁷ | GLPAPIEK | 6.63 | y₄-y₆ |
| | 1.23-6.63 | (SEQ ID NO: 71) | | |
| IgG3 | 0.61⁷ | WYVDGVEVHNAK | 1.94 | y₆, y₈, |
| | 0.16-1.94 | (SEQ ID NO: 72) | | y₉ |
| IgG4 | 0.24⁷ | TTPPVLDSDGSFFLYSR | NS | y₈, y₁₀, |
| | 0.03-1.33 | (SEQ ID NO: 73) | | y₁₂ |
| IgA1 | 1.88⁶ | TPLTATLSK | NS | y₅-y₇ |
| | 1.36-2.5 | (SEQ ID NO: 74) | | |
| IgA2 | 0.54⁶ | DASGATFTWTPSSGK* | 0.6 | y₇-y₁₀ |
| | 0.28-0.61 | (SEQ ID NO: 75) | | |
| IgA1- | 2.42 | WLQGSQELPR | 3.1 | y₆-y₈ |
| 2 | 1.64-3.11 | (SEQ ID NO: 76) | | |
| IgM | 0.70⁸ | DGFFGNPR | 2.3 | y₄-y₆ |
| | 0.4-2.3 | (SEQ ID NO: 77) | | |
| κ LC | 2.31⁹ | TVAAPSVFIFPPSDEQLK* | 3.0 | y₈, y₉, |
| | 1.55-3.08 | (SEQ ID NO: 78) | | y₁₁ |
| λ LC | 1.54⁹ | AGVETTTPSK | 2.24 | y₅-y₇ |
| | 0.83-2.24 | (SEQ ID NO: 79) | | |
| IgD | 0.0139¹⁰ | EPAAQAPVK | 0.50 | y₅-y₇ |
| | 0.001-0.024 | (SEQ ID NO: 80) | | |
| IgE | 0.0001¹¹ | GSGFFVFSR* | 0.50 | y₅-y₇ |
| | 0-0.002 | (SEQ ID NO: 81) | | |
| Albu- | 35¹¹ | LVNEVTEFAK* | 3.5 | y₅, y₇, |
| min | 30-40 | (SEQ ID NO: 82) | | y₈ |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating a BRAF inhibitor resistant or a BRAF mutant cancer in a subject, the method comprising administering to the subject a pharmaceutically effective amount of:
a) a BRAF inhibitor, and
b) a heat shock protein (HSP) inhibitor, a HSP co-chaperone inhibitor, or a combination thereof.

2. The method of claim 1, wherein the BRAF inhibitor is selected from the group consisting of vemurafenib and dabrafenib.

3. The method of claim 2, wherein the BRAF inhibitor is vemurafenib.

4. The method of claim 1, wherein the HSP inhibitor is selected from the group consisting of a HSP90 inhibitor, a HSP27 inhibitor, a HSP70 inhibitor, a HSP71 inhibitor, a HSP72 inhibitor, a HSP74 inhibitor, a HSP7C inhibitor, a HSP7E inhibitor, a HSPA5 inhibitor, a CDC37 inhibitor, and a HSPB3 inhibitor.

5. The method of claim 4, wherein the HSP inhibitor is a HSP90 inhibitor.

6. The method of claim 5, wherein the HSP90 inhibitor is selected from the group consisting of a HSP90α inhibitor, a HSP90β inhibitor, and a HSP90β2 inhibitor.

7. The method of claim 6, wherein the HSP90 inhibitor is selected from the group consisting of 5-((R)-sec-butylamino)-N1-((1R,3s,5S)-8-(5-(cyclopropanecarbonyl)pyridin-2-yl)-8-azabicyclo[3.2.1]octan-3-yl)-2-methylterephthalamide (XL888), 17-(Allylamino)-17-demethoxygeldanamycin (17-AAG), 17-Dimethylaminoethylamino-17-demethoxygeldanamycin (17-DMAG), and Ganetespib (STA-9090).

8. The method of claim 7, wherein the HSP90 inhibitor is XL888.

9. The method of claim 1, wherein the HSP co-chaperone inhibitor is selected from the group consisting of a HSP90 co-chaperone inhibitor, a HSP27 co-chaperone inhibitor, a HSP70 co-chaperone inhibitor, a HSP71 co-chaperone inhibitor, a HSP72 co-chaperone inhibitor, a HSP74 co-chaperone inhibitor, a HSP7C co-chaperone inhibitor, a HSP7E co-chaperone inhibitor, a HSPA5 co-chaperone inhibitor, a CDC37 co-chaperone inhibitor, and a HSPB3 co-chaperone inhibitor.

10. The method of claim 9, wherein the HSP co-chaperone inhibitor is a HSP90 co-chaperone inhibitor.

11. The method of claim 10, wherein the HSP90 co-chaperone inhibitor is selected from the group consisting of a HSP90α co-chaperone inhibitor, a HSP90β co-chaperone inhibitor, and a HSP90β2 co-chaperone inhibitor.

12. The method of claim 1, wherein the method comprises administering an HSP inhibitor and an HSP co-chaperone inhibitor to the subject.

13. The method of claim 1, wherein the BRAF inhibitor resistant or BRAF mutant cancer is selected from the group consisting of multiple myeloma, melanoma, lung cancer, colorectal cancer, thyroid carcinoma, blood cancer, leukemia, and lymphoma.

14. The method of claim 13, wherein the BRAF inhibitor resistant or BRAF mutant cancer is a melanoma.

15. A method of monitoring the effectiveness of a heat shock protein (HSP) or HSP co-chaperone inhibitor, the method comprising:
a) administering to a subject an HSP inhibitor or a HSP co-chaperone inhibitor;
b) determining the level of a HSP or an HSP co-chaperone in a sample from the subject, wherein the level of HSP or HSP co-chaperone is determined using liquid chromatography-multiple reaction monitoring (LC-MRM) or direct infusion-multiple reaction monitoring (DI-MRM) mass spectrometry; and
c) comparing the level of the HSP or HSP co-chaperone in the sample to a control, wherein an increase or decrease of the HSP or HSP co-chaperone as compared to a control indicates the effectiveness of the HSP or HSP co-chaperone inhibitor.

16-27. (canceled)

28. A method of predicting responsiveness of a subject with cancer to a heat shock protein (HSP) or HSP co-chaperone inhibitor, the method comprising:
a) determining the level of the HSP or the HSP co-chaperone in a tumor sample from the subject using liquid chromatography-multiple reaction monitoring (LC-MRM) or direct infusion-multiple reaction monitoring (DI-MRM) mass spectrometry; and
b) administering to the subject the HSP or HSP co-chaperone inhibitor if the HSP or HSP co-chaperone is overexpressed in the tumor sample.

29-45. (canceled)


SEQUENCE LISTING (83 sequences)

SEQ ID NO: 1 - protein, 9 aa, Artificial Sequence
  Synthetic Construct
ANELLINVK

SEQ ID NO: 2 - protein, 8 aa, Artificial Sequence
  Synthetic Construct
DIVGAVLK

SEQ ID NO: 3 - protein, 7 aa, Artificial Sequence
  Synthetic Construct
ALLFVPR

SEQ ID NO: 4 - protein, 7 aa, Artificial Sequence
  Synthetic Construct
ALLFIPR

SEQ ID NO: 5 - protein, 15 aa, Artificial Sequence
  Synthetic Construct
HSQFLGYPITLYLEK

SEQ ID NO: 6 - protein, 9 aa, Artificial Sequence
  Synthetic Construct
LQAEAQQLR

SEQ ID NO: 7 - protein, 8 aa, Artificial Sequence
  Synthetic Construct
ADLINNLR

SEQ ID NO: 8 - protein, 15 aa, Artificial Sequence
  Synthetic Construct
NQVALNPQNTVFDAK

SEQ ID NO: 9 - protein, 10 aa, Artificial Sequence
  Synthetic Construct
EIAEAYLGGK

SEQ ID NO: 10 - protein, 8 aa, Artificial Sequence
  Synthetic Construct
GTLDPVEK

SEQ ID NO: 11 - protein, 11 aa, Artificial Sequence
  Synthetic Construct
AFSDPFVEAEK

SEQ ID NO: 12 - protein, 11 aa, Artificial Sequence
  Synthetic Construct
VEIIANDQGNR

SEQ ID NO: 13 - protein, 15 aa, Artificial Sequence
  Synthetic Construct
NPDDITNEEYGEFYK

SEQ ID NO: 14 - protein, 7 aa, Artificial Sequence
  Synthetic Construct
FYEQFSK

SEQ ID NO: 15 - protein, 11 aa, Artificial Sequence
  Synthetic Construct
DQVANSAFVER

SEQ ID NO: 16 - protein, 10 aa, Artificial Sequence
  Synthetic Construct
LGIHEDSQNR

SEQ ID NO: 17 - protein, 13 aa, Artificial Sequence
  Synthetic Construct
IEADSESQEEIIR

SEQ ID NO: 18 - protein, 11 aa, Artificial Sequence
  Synthetic Construct
EQVANSAFVER

SEQ ID NO: 19 - protein, 17 aa, Artificial Sequence
  Synthetic Construct
EGEEAGPGDPLLEAVPK

SEQ ID NO: 20 - protein, 14 aa, Artificial Sequence
  Synthetic Construct
FTVLFPSGTPLPAR

SEQ ID NO: 21 - protein, 8 aa, Artificial Sequence
  Synthetic Construct
VGVNGFGR

SEQ ID NO: 22 - protein, 8 aa, Artificial Sequence
  Synthetic Construct
DDSPSLPK

SEQ ID NO: 23 - protein, 9 aa, Artificial Sequence
  Synthetic Construct
QTALVELLK

SEQ ID NO: 24 - protein, 8 aa, Artificial Sequence
  Synthetic Construct
AEFVEVTK

SEQ ID NO: 25 - protein, 7 aa, Artificial Sequence
  Synthetic Construct
YLYEIAR

SEQ ID NO: 26 - protein, 17 aa, Artificial Sequence
  Synthetic Construct
VTHAVVTVPAYFNDAQR

SEQ ID NO: 27 - protein, 12 aa, Artificial Sequence
  Synthetic Construct
ELEEIVQPIISK

SEQ ID NO: 28 - protein, 14 aa, Artificial Sequence
  Synthetic Construct
TFAPEEISAMVLTK

SEQ ID NO: 29 - protein, 12 aa, Artificial Sequence
  Synthetic Construct
TWNDPSVQQDIK

SEQ ID NO: 30 - protein, 14 aa, Artificial Sequence
  Synthetic Construct
ITPSYVAFTPEGER

SEQ ID NO: 31 - protein, 17 aa, Artificial Sequence
  Synthetic Construct
SQIFSTASDNQPTVTIK

SEQ ID NO: 32 - protein, 15 aa, Artificial Sequence
  Synthetic Construct
NQLTSNPENTVFDAK

SEQ ID NO: 33 - protein, 12 aa, Artificial Sequence
  Synthetic Construct
ADLINNLGTIAK

SEQ ID NO: 34 - protein, 15 aa, Artificial Sequence
  Synthetic Construct
HSQFLGYPITLYLEK

SEQ ID NO: 35 - protein, 9 aa, Artificial Sequence
  Synthetic Construct
YESLTDPSK

SEQ ID NO: 36 - protein, 10 aa, Artificial Sequence
  Synthetic Construct
SIYYITGESK

SEQ ID NO: 37 - protein, 14 aa, Artificial Sequence
  Synthetic Construct
EDISSIEIVGGATR

SEQ ID NO: 38 - protein, 11 aa, Artificial Sequence
  Synthetic Construct
SFDDPIVQTER

SEQ ID NO: 39 - protein, 7 aa, Artificial Sequence
  Synthetic Construct
FYEAFSK

SEQ ID NO: 40 - protein, 15 aa, Artificial Sequence
  Synthetic Construct
HLEINPDHPIVETLR

SEQ ID NO: 41 - protein, 9 aa, Artificial Sequence
  Synthetic Construct
LLQDFFNGR

SEQ ID NO: 42 - protein, 11 aa, Artificial Sequence
  Synthetic Construct
FELSGIPPAPR

SEQ ID NO: 43 - protein, 16 aa, Artificial Sequence
  Synthetic Construct
IINEPTAAAIAYGLDR

SEQ ID NO: 44 - protein, 13 aa, Artificial Sequence
  Synthetic Construct
TTPSYVAFTDTER

SEQ ID NO: 45 - protein, 7 aa, Artificial Sequence
  Synthetic Construct
LIGDAAK

SEQ ID NO: 46 - protein, 14 aa, Artificial Sequence
  Synthetic Construct
EDIYAVEIVGGATR

SEQ ID NO: 47 - protein, 15 aa, Artificial Sequence
  Synthetic Construct
EFSITDVVPYPISLR

SEQ ID NO: 48 - protein, 14 aa, Artificial Sequence
  Synthetic Construct
ELSTTLNADEAVTR

SEQ ID NO: 49 - protein, 18 aa, Artificial Sequence
  Synthetic Construct
SNLAYDIVQLPTGLTGIK

SEQ ID NO: 50 - protein, 9 aa, Artificial Sequence
  Synthetic Construct
EIAEAYLGK

SEQ ID NO: 51 - protein, 13 aa, Artificial Sequence
  Synthetic Construct
TTPSYVAFTDTER

SEQ ID NO: 52 - protein, 12 aa, Artificial Sequence
  Synthetic Construct
DAGTIAGLNVLR

SEQ ID NO: 53 - protein, 11 aa, Artificial Sequence
  Synthetic Construct
FELTGIPPAPR

SEQ ID NO: 54 - protein, 14 aa, Artificial Sequence
  Synthetic Construct
SQIHDIVLVGGSTR

SEQ ID NO: 55 - protein, 8 aa, Artificial Sequence
  Synthetic Construct
AAGFNVLR

SEQ ID NO: 56 - protein, 10 aa, Artificial Sequence
  Synthetic Construct
YEIDTGEETK

SEQ ID NO: 57 - protein, 11 aa, Artificial Sequence
  Synthetic Construct
FAQVVLQDLDK

SEQ ID NO: 58 - protein, 9 aa, Artificial Sequence
  Synthetic Construct
FVNPEDVAR

SEQ ID NO: 59 - protein, 7 aa, Artificial Sequence
  Synthetic Construct
DWYPHSR

SEQ ID NO: 60 - protein, 10 aa, Artificial Sequence
  Synthetic Construct
QLSSGVSEIR

SEQ ID NO: 61 - protein, 9 aa, Artificial Sequence
  Synthetic Construct
DGVVEITGK

SEQ ID NO: 62 - protein, 10 aa, Artificial Sequence
  Synthetic Construct
LFDQAFGLPR

SEQ ID NO: 63 - protein, 16 aa, Artificial Sequence
  Synthetic Construct
VSLDVNHFAPDELTVK

SEQ ID NO: 64 - protein, 17 aa, Artificial Sequence
  Synthetic Construct
LATQSNEITIPVTFESR

SEQ ID NO: 65 - protein, 8 aa, Artificial Sequence
  Synthetic Construct
GPSWDPFR

SEQ ID NO: 66 - protein, 7 aa, Artificial Sequence
  Synthetic Construct
VPFSLLR

SEQ ID NO: 67 - protein, 11 aa, Artificial Sequence
  Synthetic Construct
TVDNLLEVSAR

SEQ ID NO: 68 - protein, 21 aa, Artificial Sequence
  Synthetic Construct
LGPGGLDPVEVYESLPEELQK

SEQ ID NO: 69 - protein, 17 aa, Artificial Sequence
  Synthetic Construct
EGEEAGPGDPLLEAVPK

SEQ ID NO: 70 - protein, 12 aa, Artificial Sequence
  Synthetic Construct
GPSVFPLAPSSK

SEQ ID NO: 71 - protein, 8 aa, Artificial Sequence
  Synthetic Construct
GLPAPIEK

SEQ ID NO: 72 - protein, 12 aa, Artificial Sequence
  Synthetic Construct
WYVDGVEVHNAK

SEQ ID NO: 73 - protein, 17 aa, Artificial Sequence
  Synthetic Construct
TTPPVLDSDGSFFLYSR

SEQ ID NO: 74 - protein, 9 aa, Artificial Sequence
  Synthetic Construct
TPLTATLSK

SEQ ID NO: 75 - protein, 15 aa, Artificial Sequence
  Synthetic Construct
DASGATFTWTPSSGK

SEQ ID NO: 76 - protein, 10 aa, Artificial Sequence
  Synthetic Construct
WLQGSQELPR

SEQ ID NO: 77 - protein, 8 aa, Artificial Sequence
  Synthetic Construct
DGFFGNPR

SEQ ID NO: 78 - protein, 18 aa, Artificial Sequence
  Synthetic Construct
TVAAPSVFIFPPSDEQLK

SEQ ID NO: 79 - protein, 10 aa, Artificial Sequence
  Synthetic Construct
AGVETTTPSK

SEQ ID NO: 80 - protein, 9 aa, Artificial Sequence
  Synthetic Construct
EPAAQAPVK

SEQ ID NO: 81 - protein, 9 aa, Artificial Sequence
  Synthetic Construct
GSGFFVFSR

SEQ ID NO: 82 - protein, 10 aa, Artificial Sequence
  Synthetic Construct
LVNEVTEFAK

SEQ ID NO: 83 - protein, 732 aa, Artificial Sequence
  Synthetic Construct
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD